(19) United States
(12) Patent Application Publication
(10) Pub. No.: US 2026/0137630 A1
(43) Pub. Date: May 21, 2026

(54) COMPOSITIONS AND METHODS FOR T CELL TARGETED DELIVERY OF THERAPEUTIC AGENTS AND ACTIVATION OF T CELLS

(71) Applicant: The Trustees of the University of Pennsylvania, Philadelphia, PA (US)

(72) Inventors: Michael J. Mitchell, Philadelphia, PA (US); Ann Metzloff, Philadelphia, PA (US); Margaret M. Billingsley, Hockessin, DE (US)

(21) Appl. No.: 19/119,028

(22) PCT Filed: Oct. 6, 2023
(86) PCT No.: PCT/US2023/076231

§ 371 Date: Apr. 7, 2025

Related U.S. Application Data
(60) Provisional application No. 63/378,819, filed on Oct. 7, 2022.

(51) Int. Cl. A61K 9/51; A61K 31/7088; A61K 38/00; A61K 45/06; A61K 47/22; A61P 35/02; C07K 16/00; C12N 5/0783; C12N 5/10

(52) U.S. Cl. CPC A61K 9/5123; A61K 31/7088; A61K 38/00; A61K 45/06; A61K 47/22; A61P 35/02; C07K 16/00; C12N 5/0636; C12N 5/0646; C12N 5/10; C12N 2510/00

(57) ABSTRACT

The present disclosure relates, in part, to immune cell targeted lipid nanoparticle (LNP) compositions, and methods of use thereof for ex vivo delivery of nucleic acid molecules and/or therapeutic agents to a target cell. In certain embodiments, the LNPs described herein are suitable for T cell activation. In certain embodiments, the nucleic acid molecules encode chimeric antigen receptors (CARs). In certain embodiments, the present disclosure relates to the use of the LNPs described herein for the treatment, prevention, and/or amelioration of diseases and/or disorders, including but not limited to cancer.

### CROSS-REFERENCE TO RELATED APPLICATIONS

This application claims priority under 35 U.S.C. § 119(e) to U.S. Provisional Patent Application No. 63/378,819, filed Oct. 7, 2022, which is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

Chimeric antigen receptor (CAR) T cell therapy has achieved remarkable clinical success. However, producing these bespoke cancer-killing cells is a complicated ex vivo process of leukapheresis, artificial T cell activation, and CAR construct introduction.

Activation is vital for CAR construct uptake and differentiation into effector T cell phenotype. In the body, native T cells are activated when CD3/TCR and CD28 (two T cell surface molecules) engage with antigen presenting cells (APCs). Ex vivo, this process is mimicked with antibodies against CD3 and CD28, often conjugated to magnetic beads. While effective, removal of beads is cumbersome and results in loss of engineered cells.

Thus, there is a need in the art for improved LNP compositions suitable for T cell activation and delivery of cargo, including but not limited to nucleic acid cargo. The present invention addresses and satisfies this unmet need.

### BRIEF SUMMARY

In one aspect, the present disclosure provides an immune cell targeted lipid nanoparticle (LNP). In certain embodiments, the LNP comprises at least one ionizable lipid. In certain embodiments, the LNP comprises at least one neutral lipid. In certain embodiments, the LNP comprises cholesterol and/or a modified derivative thereof. In certain embodiments, the LNP comprises at least one polymer conjugated lipid and/or a modified derivative thereof. In certain embodiments, the LNP comprises a domain specific to binding a surface molecule of a target cell. In certain embodiments, the cell targeting domain is covalently conjugated to at least one component of the LNP.

In certain embodiments, the at least one ionizable lipid comprises a compound of Formula (I), or a salt, solvate, stereoisomer, or isotopologue thereof, wherein R^{1a}, R^{1b}, R^{1a}, R^{2b}, R^{2c}, R^{2d}, R^{2e}, R^{2f}, R^{2g}, and R^{2h} are defined elsewhere herein:

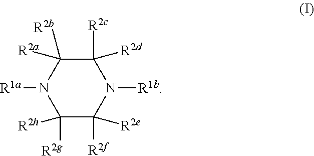

In certain embodiments, the at least one polymer conjugated lipid and/or modified derivative thereof comprises at least one polymer conjugated lipid and at least one modified derivative thereof. In certain embodiments, the modified derivative of the polymer conjugated lipid is a compound of Formula (II), or a salt, solvate, stereoisomer, or isotopologue thereof, wherein R^{5a}, R^{5b}, Z, L², and D_{ct} are defined elsewhere herein:

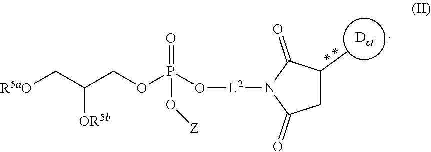

In one aspect, the present disclosure provides a lipid nanoparticle (LNP). In certain embodiments, the LNP comprises at least one ionizable lipid. In certain embodiments, the LNP comprises at least one neutral lipid. In certain embodiments, the LNP comprises at least one cholesterol compound and/or modified derivative thereof. In certain embodiments, the LNP comprises at least one polymer conjugated lipid and at least one compound of Formula (II), or a salt, solvate, stereoisomer, or isotopologue thereof.

In one aspect, the present disclosure provides a lipid nanoparticle (LNP). In certain embodiments, the LNP comprises at least one ionizable lipid compound of Formula (I), or a salt, solvate, stereoisomer, or isotopologue thereof. In certain embodiments, the LNP comprises at least one neutral lipid. In certain embodiments, the LNP comprises at least one cholesterol compound and/or modified derivative thereof. In certain embodiments, the LNP comprises at least one polymer conjugated lipid. In certain embodiments, the LNP comprises at least one cell targeting domain specific to binding a surface molecule of a target cell. In certain embodiments, the the cell targeting domain is covalently conjugated to at least one component of the LNP.

In one aspect, the present disclosure provides a lipid nanoparticle (LNP). In certain embodiments, the LNP comprises at least one ionizable lipid compound of Formula (I), or a salt, solvate, stereoisomer, or isotopologue thereof. In certain embodiments, the LNP comprises at least one neutral lipid. In certain embodiments, the LNP comprises at least one cholesterol compound and/or modified derivative thereof. In certain embodiments, the LNP comprises at least one polymer conjugated lipid and at least one compound of Formula (II), or a salt, solvate, stereoisomer, or isotopologue thereof.

In one aspect, the present disclosure provides a method of treating, preventing, and/or ameliorating cancer in a subject, the method comprising administering to the subject the lipid nanoparticle (LNP) of the present disclosure and/or the pharmaceutical composition of the present disclosure.

In one aspect, the present disclosure provides a method of preparing a modified immune cell or precursor thereof, comprising contacting an immune cell or precursor thereof with at least one lipid nanoparticle (LNP) of the present disclosure.

In certain embodiments, the modified immune cell or precursor cell thereof is selected from the group consisting of an αβ T cell, a γδ T cell, a CD8+ T cell, a CD4+ helper T cell, a CD4+ regulatory T cell, an NK T cell, an NK cell, and any combination thereof.

In certain embodiments, the modified immune cell or precursor thereof is a T cell. In certain embodiments, the T cell is a CD4+ T cell. In certain embodiments, the T cell is a CD8+ T cell.

### BRIEF DESCRIPTION OF THE DRAWINGS

The following detailed description of preferred embodiments of the invention will be better understood when read in conjunction with the appended drawings. For the purpose of illustrating the invention, non-limiting embodiments are illustrated in the drawings. It should be understood, however, that the invention is not limited to the precise arrangements and instrumentalities of the embodiments shown in the drawings.

FIG. 1 provides a schematic depicting antigen presenting cell mimetic activating LNPs (aLNPs) rapidly activating primary human T cells and transfecting them with CAR mRNA in a single step. In the body. T cells are activated when they engage with antigen presenting cells (APCs). For complete activation, APCs must provide T cells with a primary and a costimulatory signal. The primary signal occurs when APC peptide-MHC interacts with T cell CD3/TCR. The costimulatory signal occurs when APC CD80/CD86 interacts with T cell CD28. Once activated, the T cell can carry out its effector function in the body. To engineer chimeric antigen receptor (CAR) T cells outside of the body with lipid nanoparticles (LNPs), T cells must first be activated. Antibodies against CD3 and CD28, often conjugated to magnetic beads, are used to mimic APC activation before dosing the T cells with mRNA LNPs. Activating LNPs (aLNPs) have been prepared by conjugating CD3 and CD28 antibody fragments to the surfaces of the LNPs described herein. aLNPs combine the activating properties of the beads and the mRNA-delivering capabilities of traditional LNPs, enabling activation of and CAR mRNA delivery to T cells in a single, rapid step.

FIGS. 2A-2D: formulation and characterization of activating LNPs (aLNPs). FIG. 2A: molar composition of maleimide-LNPs (mal-LNPs). PEG, polyethylene glycol. DOPE, dioleoylphosphatidylethanolamine. C14-4, an ionizable lipid. FIG. 2B: S_{N}2 synthesis of the ionizable lipid C14-4 from 1,2-epoxytetradecane and a polyamine core. FIG. 2C: formulation of maleimide-LNPs (mal-LNPs) by microfluidic mixing, the cleavage and reduction of antibody fragments, and the conjugation of antibody fragments onto the mal-LNP surface to generate aLNPs. FIG. 2D: hydrodynamic diameter (intensity weighted z-average) distributions of mal-LNPs and 1:1 anti-CD3:anti-CD28 aLNPs (top); and z-average and PDI measurements, collected in triplicate (bottom). Reported values are average z-average±the calculated standard deviation (calculated standard deviation=sqrt(average PDI×average z-average²)) and average PDI±the standard deviation of the three PDI measurements.

FIG. 3 provides an image depicting denaturing gel electrophoresis (4-12% Bis-Tris gel with MES-SDS running buffer) of anti-human CD3 and anti-human CD28 antibodies (αCD3, αCD28) before and after treatment with IdeZ protease and dithiothreitol (DTT). Following treatment with IdeZ, cleavage of ˜25 kDa Fc fragments to generate F(ab′)₂ fragments is apparent for both aCD3 and aCD28. Following treatment with DTT, the αCD3 F(ab′)2 fragments appear to be almost completely reduced into separate heavy (Fd′) and light chain (LC) fragments both of molecular weight 25 kDa. The αCD28 F(ab′)₂ fragments also appear to be almost completely reduced into separate heavy (Fd′) and light chain (LC) fragments of molecular weight 25 kDa following treatment with DTT. However, the light band at 50 kDa for αCD28+IdeZ+DTT indicates that some heavy and light chain fragments remain bound together as F(ab′) fragments. Materials: Bolt™ 4 to 12%, Bis-Tris, 1.0 mm, Mini Protein Gels (Invitrogen, #NW04120BOX); 20X Bolt™ MES SDS Running Buffer (Invitrogen, #B0002); 4X Bolt™ LDS Sample Buffer (Invitrogen, #B0007); PageRuler™ Plus Prestained Protein Ladder, 10 to 250 kDa (Thermo Scientific, #26619).

FIGS. 4A-4C: aLNPs efficiently transfect primary human T cells with luciferase mRNA in the absence of activating beads. Luminescence in primary human T cells dosed with one of five mRNA lipid nanoparticles (LNPs) (FIG. 4A) and exposed to one of three treatment conditions (FIG. 4B). Each bar represents the mean of data collected for three different donors and normalized to untreated cells within each donor (FIG. 4C). On each bar, the mean normalized luminescence for each donor is plotted as a shape (circle, triangle, or rhombus) to highlight donor-to-donor variability. Differences in LNP means within each treatment were assessed with a two-way repeated measures ANOVA with post hoc t tests using Tukey's correction for multiple comparisons. Only results of comparisons to aLNPs are shown. n=3 donors, with n=3 replicates per donor. Data are presented as mean±SD. *p≤0.05, ns=not significant. Donor 1 (age: 28; sex: female); Donor 2 (age: 27: sex: male); and Donor 3 (age: 31; sex: female).

FIGS. 5A-5F: The ratio of CD3 to CD28 antibody fragments on the aLNP surface influences the number and mean fluorescence intensity (MFI) of transfected cells. FIG. 5A: the control treatment (top) and the various aLNPs (bottom) given to primary human T cells in the panels below. FIG. 5B: representative flow cytometry histograms obtained from primary human T cells treated as in panel a with mCherry mRNA. mCherry+ cells are defined as those to the right of the dashed line. FIG. 5C: percentage of single cells mCherry+ after each treatment, from the same experiment as the representative histograms. FIG. 5D: MFI of mCherry+ single cells after each treatment, from the same experiment as the representative histograms. n=1 donor, with n=3 replicates. FIG. 5E: repeat of the experiment in FIGS. 5B-5D using mRNA encoding EGFP to confirm results; percentage of single cells EGFP+ after each treatment (top); MFI of EGFP+ single cells after each treatment (bottom). n=1 donor, with n=4 replicates. FIG. 5F: viability/expansion of primary human T cells treated as in panel a. Each bar represents the mean of data collected for three donors and normalized to untreated cells within each donor. On each bar, the mean for each donor is plotted as a shape (inverted triangle, square, or hexagon) to highlight donor-to-donor variability. n=3 donors, with n=3 replicates per donor. In FIGS. 5B-5E, for each bar graph, differences between group means were assessed by an ordinary one-way ANOVA with post hoc t tests using Tukey's correction for multiple comparisons. Only comparisons to B+L are shown. In FIG. 5F, differences between all group means were assessed by a repeated measures one-way ANOVA with post hoc t tests using Tukey's correction for multiple comparisons. Not shown=not significant. For FIGS. 5B-5F, data are presented as mean±SD. *p≤0.05, **p≤0.01, ***p≤0.001, ****p≤0.0001, ns=not significant. FIGS. 5B-5D: Donor (age: 58; sex: female); FIG. 5E: Donor (age: 31; sex: male); FIG. 5F: Donor 1 (age: 33; sex: female); Donor 2 (age: 31: sex: male); Donor 3 (age: 37; sex: male).

FIG. 6: flow cytometry gating strategy used to confirm expression of proteins (mCherry, EGFP, and anti-human CD19 CAR) encoded by aLNP-delivered mRNA. Cells were gated for lymphocytes, single cells, the mRNA encoded protein, and (in some cases) CD4 and CD8.

FIG. 7: side scatter (SSC, a measure of cell complexity) vs. forward scatter (FSC, a measure of cell size) for untreated primary human T cells, bead+mal-LNP treated primary human T cells, and 1:10 aLNP treated primary human T cells, as measured by flow cytometry. Similar increases in SSC and FSC over untreated cells are visible for bead+mal-LNP treated cells and 1:10 aLNP treated cells, qualitatively indicating that the two treatments induce similar levels of activation.

FIGS. 8A-8F: anti-CD19 CAR T cells generated with aLNPs perform potent cancer cell killing ex vivo, and express cytokines and cell-surface activation markers at levels similar to those for bead-activated T cells. FIGS. 8A-8C: CAR T cell and Nalm6 leukemia cell co-culture assay. FIG. 8A: co-culture assay plating set up and in vitro transcribed anti-CD19 CAR mRNA. FIG. 8B: representative flow cytometry histograms obtained from primary human T cells that received no treatment (NT), beads+mal-LNPs (B+L), or 1:10 aLNPs (1:10). CAR+ cells are defined as those to the right of the dashed line. FIG. 8C: percentage of Nalm6 cancer cells killed when cultured with different ratios of CAR T cells. n=1 donor, with n=3 replicates. FIG. 8D: percentage of cells expressing CD69, a cell-surface activation marker, for CD4+ and CD8+ T cells that received NT, B+L, or 1:10 aLNPs. n=1 donor, with n=3 replicates. FIG. 8E: results of ELISAs for tumor necrosis factor alpha (TNFα) and interferon gamma (IFNγ) secretion by primary human T cells that received NT, B+L, or 1:10 aLNPs. n=1 donor, with n=2 replicates. FIG. 8F: viability/expansion of primary human T cells treated with escalating doses of 1:10 aLNPs. n=1 donor, with n=3 replicates. For FIGS. 8A-8C, differences in treatment means within each CAR T cell:cancer cell ratio were assessed by a two-way ANOVA with post hoc t tests using Sidak's correction for multiple comparisons. For FIGS. 8D-8F, for each graph, differences between all group means were assessed by an ordinary one-way ANOVA with post hoc t tests using Tukey's correction for multiple comparisons. All data are presented as mean±SD. *p≤0.05, **p≤0.01, ***p≤0.001, ****p≤0.0001. Not shown=not significant. FIGS. 8A-8C: Donor (age: 31; sex: female); FIG. 8D: Donor (age: 58; sex: female); FIG. 8E: Donor (age: 28; sex: female); FIG. 8F: Donor (age: 23; sex: male).

FIG. 9: flow cytometry plot showing the percentage of anti-human CD19 CAR+ primary human T cells treated with 1:10 aLNPs that are CD4+ vs. CD8+. Cells were stained with a rabbit anti-mouse FMC63 scFv monoclonal antibody conjugated to PE (Cytoart, #200105), an Alexa FluorX 700 mouse anti-human CD8 antibody (Biolegend, #344724), and an eFluor™ 450 mouse anti-human CD4 antibody (Life Technologies, #48-0049-42).

FIG. 10: Flow cytometry histograms and percentages of CAR+ cells for each dose of 1:10 aLNP generated anti-human CD19 CAR T cells administered during the in vivo leukemia xenograft murine model (FIG. 6). Cells were stained with a rabbit anti-mouse FMC63 scFv monoclonal antibody conjugated to PE (Cytoart, #200105). 2×10⁶ CAR+ cells were administered to each mouse for each dose.

FIGS. 11A-11D: Adoptive transfer of anti-CD19 CAR T cells generated with aLNPs reduces tumor burden in a xenograft mouse model of leukemia. FIG. 11A: schedule used to establish a low-leukemic burden in NSG mice followed by repeated treatments with CAR T cells generated with 1:10 aLNPs. FIG. 11B: time-course IVIS images ofNalm6 (luciferase-expressing human leukemia) tumor-bearing NSG mice treated with PBS, untransfected T cells, or 1:10 aLNP generated CAR T cells. FIG. 11C: time-course of quantification of average total flux per mouse for the images shown in panel b. FIG. 11D: Kaplan-Meier survival curves of the mice following treatment. FIGS. 11B-11D represent data from a single experiment, for which n=5 mice/group. For FIG. 11C, data are presented as mean SD. Differences between all treatment means within each day were assessed by a two-way repeated measures ANOVA with post hoc t tests using Tukey's correction for multiple comparisons. *p≤0.05, ns=not significant. For FIG. 11D, differences between survival profiles were assessed using pairwise Log-rank tests with Bonferroni corrections for multiple comparisons. To determine significance, the p values shown were compared to the Bonferroni-corrected a value of 0.0167. * indicates significance, ns=not significant. Donor (age: 55; sex: female).

FIG. 12 shows that 1:10 formulated aLNPs drive more rounds of cell division than traditional activating beads at 6 days post-treatment. On day 0, prior to treatment with beads +mal-LNPs or 1:10 aLNPs (400 ng CAR mRNA/60,000 primary human T cells, bead:cell ratio 1:1), primary human T cells were stained with CellTrace™ Far Red dye according to manufacturer protocol. On Days 2, 4, and 6, cells were assessed via flow cytometry. Each proliferative generation appears as a distinct leftward-shifted peak in the flow cytometry histogram, with 1:10 aLNP treatment resulting in more generations at Day 6 than beads+mal-LNPs.

FIGS. 13A-13B: CAR T cell and Nalm6 leukemia cell co-culture assays performed with T cells from two different donors. For each donor, the graph shows the percentage of Nalm6 cancer cells killed when cultured with different ratios of CAR T cells generated with beads+mal-LNPs or 1:10 aLNPs. For each graph, differences between all groups means were assessed by an ordinary one-way ANOVA with post hoc t tests using Tukey's correction for multiple comparisons. n=3 replicates per donor. Data are presented as mean+SD. *p≤0.05, **p≤0.01, ***p≤0.001, ****p≤0.0001. Not shown=not significant.

FIGS. 14A-14E show that treatment with activating beads+mal-LNPs versus 1:10 aLNPs induces similar states of T cell activation. Percentages of primary human T cells expressing CD25 (FIG. 14A), CD69 (FIG. 14B), CD44 (FIG. 14C), CD45RA (FIG. 14D), CCR7 (FIG. 14E), as assessed via flow cytometry, for cells that received no treatment (NT), LNPs with only anti-CD3 antibody fragments on their surfaces (αCD3 LNPs), LNPs with only anti-CD28 antibody fragments on their surfaces (αCD28 LNPs), beads+mal-LNPs (B+L), or 1:10 aLNPs (400 ng CAR mRNA/60,000 primary human T cells for all LNPs, for B+L, bead:cell ratio 1:1). T cell activation is indicated by upregulation of CD25, CD69, and CD44, and downregulation of CD45RA and CCR7. For each graph, differences between all group means were assessed by an ordinary one-way ANOVA with post hoc t tests using Tukey's correction for multiple comparisons. Only results of comparisons to aLNPs are shown. n=1 donor, with n=4 replicates. Data are presented as mean±SD. ns=not significant. *p≤0.05, **p≤0.01, ***p≤0.001, ****p≤0.0001.

FIGS. 15A-15B show that adoptive transfer of anti-CD19 CAR T cells generated with aLNPs reduces tumor burden in a xenograft mouse model of leukemia more effectively than traditional lentiviral CAR T cells. FIG. 15A: time-course IVIS images of Nalm6 (luciferase-expressing human leukemia) tumor-bearing NSG mice treated with PBS, a single dose of 1×10⁶ lentiviral CAR T cells on day 0 (DO), or 2×10⁶ 1:10 aLNP generated transient CAR T cells on D0, D3, and D6. FIG. 15B: time-course of quantification of average total flux per mouse for the IVIS images. FIGS. 15A-15B represent data from a single experiment, for which n=3 mice/group. In FIG. 15B, data are presented as mean±SD.

### DETAILED DESCRIPTION

The present invention relates to compositions comprising an LNP comprising at least one nucleic acid and/or therapeutic agent for the treatment of a disease or disorder, wherein the LNP is formulated for targeted delivery to an immune cell.

### Definitions

The term “about” as used herein can allow for a degree of variability in a value or range, for example, within 10%, within 5%, or within 1% of a stated value or of a stated limit of a range, and includes the exact stated value or range.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)=CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkvl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein, refer to a divalent radical of the moiety corresponding to the base group (e.g., heteroalkyl, cycloalkyl, and/or heterocycloalkyl). A divalent radical possesses two open valencies at any position(s) of the group, wherein each radical may be on a carbon atom or heteroatom. Thus, the divalent radical may form a single bond to two distinct atoms or groups, or may form a double bond with one atom.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines. N-glutarylphosphatidylethanolamines, lysylphosphatidylglycerols, palmitoyloleyolphosphatidylglycerol (POPG), and other anionic modifying groups joined to neutral lipids.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “amine” as used herein refers to primary, secondary, and tertiary amines having, e.g, the formula N(group)₃ wherein each group can independently be H or non-H, such as alkyl, aryl, and the like. Amines include but are not limited to R—NH₂, for example, alkylamines, arylamines, alkylarylamines: R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines. N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “monovalent cation” as used herein refers to any positively charged (+1) organic or inorganic ion. Non-limiting examples include H⁺, NH₄⁺, Li⁺. Na⁺, K⁺, Cu⁺, Ag⁺, Cs⁺, and Au⁺.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cationic lipids comprise a protonatable tertiary amine (e.g., pH titratable) head group, Cis alkyl chains, ether linkages between the head group and alkyl chains, and 0 to 3 double bonds. Such lipids include, e.g., DSDMA, DLinDMA, DLenDMA, and DODMA.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol. PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization. ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule.

The term “heteroaryl” as used herein refers to aromatic ring compounds containing 5 or more ring members, of which, one or more is a heteroatom such as, but not limited to, N, O, and S: for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl. 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl. 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl. 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein.

The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “local delivery.” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol. PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “monovalent” as used herein refers to a substituent connecting via a single bond to a substituted molecule. When a substituent is monovalent, such as, for example, F or Cl, it is bonded to the atom it is substituting by a single bond.

The term “mRNA” or “messenger RNA” as used herein refers to a ribonucleic acid sequences which encodes a peptide or protein. In certain embodiments, the mRNA may comprise a “transcript” that is produced by using a DNA template and encodes a peptide or protein. Typically, mRNA comprises 5′-UTR, protein coding region and 3′-UTR. mRNA can be produced by in vitro transcription from a DNA template. Methods of in vitro transcription are known to those of skill in the art. For example, various in vitro transfer kits are commercially available. According to the present invention, mRNA can be modified by further stabilizing modifications and cap formation in addition to the modifications according to the invention.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl. PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The terms “patient.” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “siRNA” or “small interfering RNA” as used herein refers to a small (e.g. generally less than 30 nucleotides) non-coding RNA molecule which functions in transcriptional and post-transcriptional regulation of gene expression. Generally, a siRNA specifically targets 1 nucleic acid. In general, a siRNA comprises a double-stranded RNA molecule that ranges from about 15 to about 29 nucleotides in length. In some embodiments, the siRNA may be 16, 17, 18, 19, 20, 21, 22, 23, 24, 25, 26, 27, 28 or 29 nucleotides in length. In some embodiments, the siRNA may be less than 16, 17, 18, 19, 20, 21, 22, 23, 24, 25, 26, 27, 28 or 29 nucleotides in length. In some embodiments, the siRNA may be more than 16, 17, 18, 19, 20, 21, 22, 23, 24, 25, 26, 27, 28 or 29 nucleotides in length. A siRNA may optionally further comprise one or two single-stranded overhangs. e.g., a 5′ overhang on one or both ends, a 3′ overhang on one or both ends, or a combination thereof. The siRNA may be formed from two RNA molecules that hybridize together or, alternatively, may be generated from a short hairpin RNA (shRNA). In some embodiments, the two strands of the siRNA may be completely complementary, such that no mismatches or bulges exist in the duplex formed between the two sequences. In other embodiments, the two strands of the siRNA may be substantially complementary, such that one or more mismatches and/or bulges may exist in the duplex formed between the two sequences. In certain embodiments, one or both of the 5′ ends of the siRNA may have a phosphate group, while in other embodiments one or both of the 5′ ends lack a phosphate group. In other embodiments, one or both of the 3′ ends of the siRNA may have a hydroxyl group, while in other embodiments one or both of the 5′ ends lack a hydroxyl group. Typically, siRNAs are targeted to exonic sequences of the target nucleic acid. One strand of the siRNA, which is referred to as the “antisense strand” or “guide strand,” includes a portion that hybridizes with a target nucleic acid. A target nucleic acid refers to a nucleic acid sequence expressed by a cell for which it is desired expression be disrupted. In the context of a therapeutic composition of the invention, disrupting expression of a target nucleic acid may produce a beneficial effect. Those of skill in the art are familiar with programs, algorithms, and/or commercial services that design siRNAs for target genes. For example, the Rosetta siRNA Design Algorithm (Rosetta Inpharmatics, North Seattle, Wash.). MISSION(R siRNA (Sigma-Aldrich, St. Louis, Mo.) and siGENOME siRNA (Thermo Scientific) may be used.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth: malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol: esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985. Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “room temperature” as used herein refers to a temperature of about 15° C., to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In one embodiment, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat.” “treating” and “treatment.” as used herein, means reducing the frequency or severity with which symptoms of a disease or condition are experienced by a subject by virtue of administering an agent or compound to the subject.

### DESCRIPTION

The first chimeric antigen receptor (CAR) T cell therapy was approved by the U.S. Food and Drug Administration (FDA) in 2017 for the treatment of relapsed or refractory acute lymphoblastic leukemia. Since then, five additional CAR T cell therapies have been approved for the treatment of hematological malignancies. This success has spurred the development of CAR T cell therapies for the treatment of solid tumors and non-malignant diseases. Currently, the FDA-approved CAR T cells are autologous, meaning that they are produced from a patient's own T cells that have been engineered to express the CAR construct. This synthetic receptor fuses a monoclonal antibody against a disease target with intracellular stimulatory and costimulatory domains to achieve both specificity and potency in cancer cell killing, respectively. To produce CAR T cells, a patient's T cells are harvested via leukapheresis. The isolated T cells are activated, viral vectors are employed to incorporate genetic constructs encoding for CAR into the genomes of the T cells, and the CAR T cells are expanded in bioreactors before reinfusion into patients.

Although CAR T cells produced in this way can bring about durable cancer remission, two serious side effects (i.e., cytokine release syndrome and neurotoxicity) are common. These toxicities generally occur within days of CAR T cell administration and can be treated with interleukin-6 receptor monoclonal antibodies and corticosteroids. Additionally, CAR T cells also persist in the body for years, exerting their targeting effects long after a patient's cancer has been cleared. In the context of CAR T cells for hematological malignancies, this leads to B cell aplasia and hypogammaglobulinemia. These adverse effects have led to the exploration of alternative, non-viral methods of CAR T cell engineering, such as the delivery of CAR-encoding messenger RNA (mRNA) to T cells. mRNA does not integrate into the genome, so it results in only transient CAR expression, which may aid in preventing the long-term side effects of CAR T cell therapy. Additionally, non-viral delivery methods could reduce manufacturing costs, increase cargo capacity, and increase safety. Therefore, mRNA CAR T cell therapy is being explored for the treatment of a variety of cancers.

In preclinical studies, mRNA CAR T therapy has been found to be as effective as viral CAR T therapy at lowering short-term cancer burden with less inherent toxicity, which has resulted in the initiation of several clinical trials. In these clinical trials, CAR mRNA was delivered to patients' isolated T cells ex vivo by electroporation, a method where electric pulses are used to generate transient pores in the cell membrane. However, electroporation requires specialized equipment and results in high rates of cell death as well as altered gene expression in the surviving cell population. An alternative approach is to encapsulate CAR mRNA in lipid or polymer nanoparticles. Nanoparticles do not require specialized equipment for cellular delivery and can be engineered to stabilize their mRNA cargo, enhance intracellular delivery, and reduce cytotoxicity compared to electroporation.

Ionizable lipid nanoparticles (LNPs) are one of the most clinically advanced nanoparticle platforms. Their successful use as the carrier for the COVID-19 mRNA vaccines validated their potency and safety in millions of patients around the world. Additionally, they can be rapidly produced at large scales. Previous work relating to development and optimization of a LNP platform for CAR mRNA delivery to primary human T cells has demonstrated its superiority over electroporation.

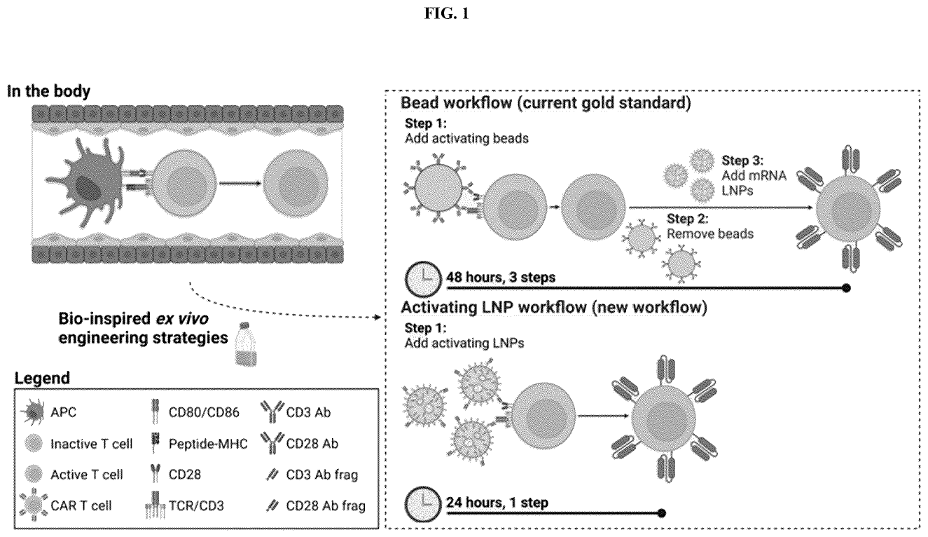

Still, in order to take up LNPs, T cells must be activated. In the body, T cells are activated when they interact with antigen presenting cells (APCs). A primary activation signal is provided when major histocompatibility complex proteins, displaying antigens, on an APC interact with CD3/T cell receptor (TCR) protein complexes on a T cell. However, for full activation of a T cell, a costimulatory activation signal must also be provided. This occurs when CD80 or CD86 proteins on the APC interact with CD28 proteins on the T cell (FIG. 1). To engineer T cells ex vivo, this process is mimicked with antibodies against CD3 and CD28, which are often attached to either magnetic beads for easy removal or to APC mimicking platforms.

In a traditional LNP administration workflow, the activating beads are added to T cells in culture. After waiting 24 hours for activation, the beads are removed with a magnet, and then mRNA LNPs are added (FIG. 1). Though this strategy is effective, it increases the time and complexity of the workflow while decreasing cell yields during bead extraction.

Thus, in one aspect, the present disclosure relates to the development of methods for T cell activation without the use of magnetic beads in the mRNA CAR T cell engineering workflow, such that mRNA CAR T cells can be produced in a single, rapid step.

In one aspect, the present disclosure relates to the hypothesis that directly conjugating CD3 and CD28 antibody fragments to the surface of LNPs could bypass the need for pretreatment with activating beads to engineer mRNA CAR T cells. In non-limiting, exemplary embodiments, as described in detail herein, thiol-maleimide chemistry was utilized to conjugate CD3 and CD28 antibody fragments to the surfaces of previously optimized T cell LNPs. The resultant “activating LNPs” or “aLNPs” mimic the activating function of APCs (FIG. 1).

The present disclosure first demonstrates that aLNPs efficiently transfect primary human T cells with mRNA in the absence of activating beads. The present disclosure further describes optimization of the ratio of CD3 to CD28 antibody fragments conjugated to the aLNP surface. Next, it was demonstrated herein that anti-CD19 CAR T cells generated with aLNPs perform potent cancer cell killing ex vivo and express cytokines and cell-surface activation markers at levels comparable to those for bead-activated T cells. Additionally, it is demonstrated herein that adoptive transfer of anti-CD19 CAR T cells generated with aLNPs reduces tumor burden in a xenograft murine model of leukemia, validating aLNPs as a platform to more efficiently produce a functional mRNA CAR T cell therapy.

The present invention relates to compositions comprising immune cell targeted LNP molecules formulated for in vivo stability and methods of use thereof for ex vivo delivery of an encapsulated agent to an immune cell. Exemplary agents that can be encapsulated in the compositions of the invention include, but are not limited to, diagnostic agents, detectable agents, and therapeutic agents. In some embodiments, the encapsulated agent comprises an agent for directing a target immune cell to a pathogen or tumor cell of interest. In certain embodiments, the present invention provides a composition comprising an immune cell targeted LNP molecule encapsulating a nucleic acid molecule encoding a CAR molecule specific for binding to an antigen on the cell surface of a pathogen or tumor cell of interest.

In one aspect, the present invention relates to T cell activating LNPs, wherein the LNPs comprise covalently conjugated antibodies against certain T cell surface proteins (e.g., CD3 and/or CD28). Certain non-limiting, exemplary LNPs comprising ionizable lipid compounds of formula (I) are provided herein. The skilled artisan appreciates that the teachings of the present disclosure may be applied to a range of LNPs comprising diverse ionizable lipids, neutral lipids, cholesterol or modified derivatives thereof, and polymer conjugated lipids.

### Lipids

In one aspect, the present disclosure provides an ionizable lipid of Formula (I), or a salt, solvate, stereoisomer, or isotopologue thereof:

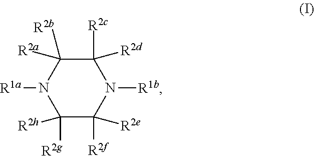

wherein:
R^{1a} and R^{1b} are each independently

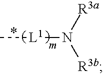

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H; R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L¹ is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

In certain embodiments, at least one selected from the group consisting of R^{2a}, R^{2b}. R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H.

In certain embodiments, at least seven selected from the group consisting of R², R^{2b}, R^{2c}, R^{2d}, R^{2e}, R^{2f}, R^{2g}, and R^{2h} are H. In certain embodiments, each of R², R^{2b}, R^{2c}, R^{2d}, R^{2e}, R^{2f}, R^{2g}, and R^{2h} are H.

In certain embodiments, L¹ is —CH₂—. In certain embodiments, L¹ is —(CH₂)₂—. In certain embodiments, L¹ is —(CH₂)₃—. In certain embodiments, L¹ is —(CH₂)₁₀—. In certain embodiments, L¹ is —(CH₂)₂O—. In certain embodiments, L¹ is —(CH₂)₃O—. In certain embodiments, L¹ is —CH₂CH(OR⁵)CH₂—. In certain embodiments, L¹ is —(CH₂)₂NR^{3c}—. In certain embodiments, L¹ is

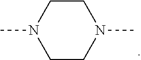

In certain embodiments, L¹ is

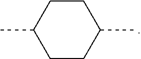

In certain embodiments, L¹ is

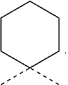

For instances of L which are asymmetric (e.g., —(CH₂)₃O—) it is understood that the present disclosure encompasses both possible orientations (e.g., —(CH₂)₃O— and —O(CH₂)₃—).

In certain embodiments, the ionizable lipid of Formula (I) is:

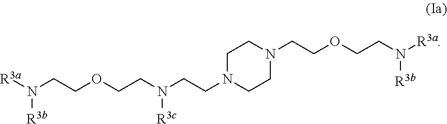

In certain embodiments, the ionizable lipid of Formula (I) is:

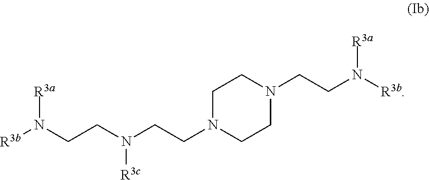

In certain embodiments, the ionizable lipid of Formula (I) is:

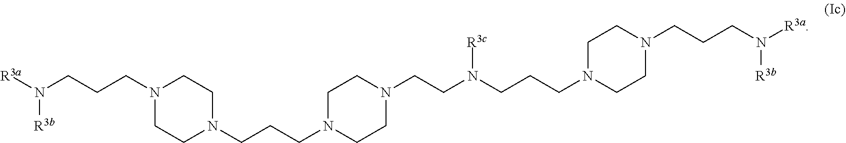

In certain embodiments, the ionizable lipid of Formula (I) is:

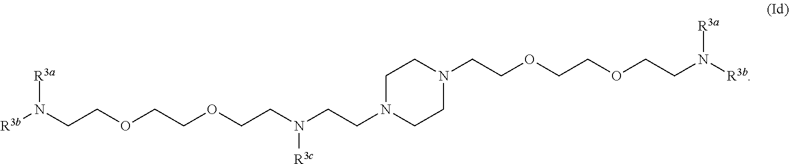

In certain embodiments, the ionizable lipid of Formula (I) is:

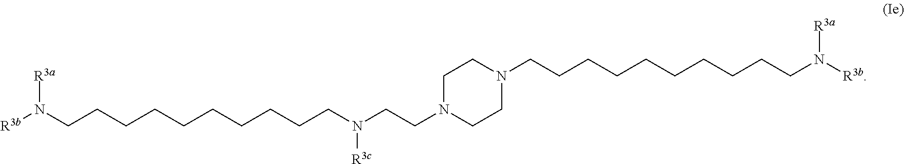

In certain embodiments, the ionizable lipid of Formula (I) is:

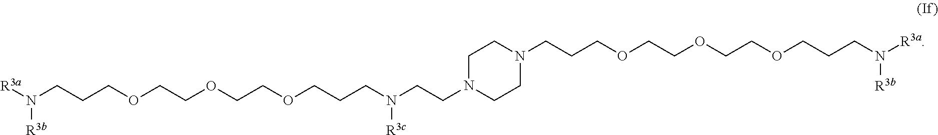

In certain embodiments, the ionizable lipid of Formula (I) is:

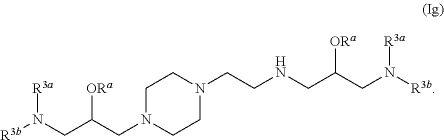

In certain embodiments, the ionizable lipid of Formula (I) is:

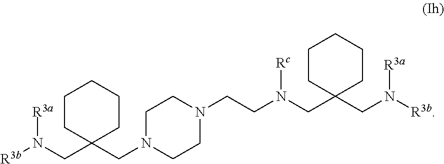

In certain embodiments, the ionizable lipid of Formula (I) is:

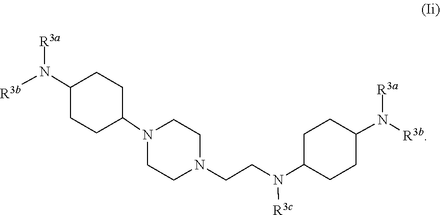

In certain embodiments, R^{3a} is H. In certain embodiments, R^{3a} is —CH₂CH(OH)(optionally substituted C₁-C₂₈ alkyl). In certain embodiments, R^{3a} is —CH₂CH(OH)(optionally substituted C₂-C₂₈ alkenyl). In certain embodiments, R^{3a} is —CH₂CH₂C(═O)O (optionally substituted C₁-C₂₈ alkyl). In certain embodiments, R^{3a} is —CH₂CH₂C(═O)NH (optionally substituted C₁-C₂₈ alkyl). In certain embodiments, R^{3b} is H. In certain embodiments, R^{3b} is —CH₂CH(OH)(optionally substituted C₁-C₂₈ alkyl). In certain embodiments, R^{3b} is —CH₂CH(OH)(optionally substituted C₂-C₂₈ alkenyl). In certain embodiments, R^{3b} is —CH₂CH₂C(═O)O (optionally substituted C₁-C₂₈ alkyl). In certain embodiments, R^{3b} is —CH₂CH₂C(═O)NH (optionally substituted C₁-C₂₉ alkyl). In certain embodiments, R^{3c} is H. In certain embodiments, R^{3c} is —CH₂CH(OH)(optionally substituted C₁-C₂₈ alkyl). In certain embodiments, R^{3c} is —CH₂CH(OH)(optionally substituted C₂-C₂₈ alkenyl). In certain embodiments. R^{3c} is —CH₂CH₂C(═O)O (optionally substituted C₁-C₂₈ alkyl). In certain embodiments, R^{3c} is —CH₂CH₂C(═O)NH (optionally substituted C₁-C₂₈ alkyl).

In certain embodiments, R^{3a} is —CH₂CH(OH)(CH₂)₉CH₃. In certain embodiments, R^{3a} is —CH₂CH(OH)(CH₂)₁CH₃. In certain embodiments, R^{3a} is —CH₂CH(OH)(CH₂)₁₃CH₃. In certain embodiments, R^{3b} is —CH₂CH(OH)(CH₂)₉CH₃. In certain embodiments, R^{3b} is —CH₂CH(OH)(CH₂)CH₃. In certain embodiments, R^{3b} is —CH₂CH(OH)(CH₂)₁₃CH₃. In certain embodiments, R^{3c} is —CH₂CH(OH)(CH₂)₉CH₃. In certain embodiments, R^{3b} is —CH₂CH(OH)(CH₂)₁₁CH₃. In certain embodiments, R³, is —CH₂CH(OH)(CH₂)₁₃CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the ionizable lipid of Formula (I) is:

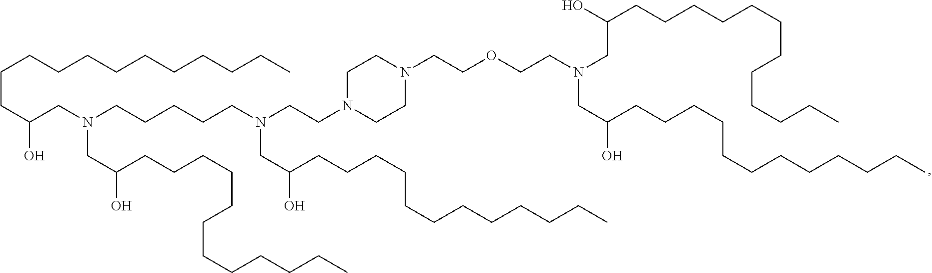 [SMILES: CCCCCCCCCCCCC(O)CN(CCCCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol) (C14-494).

### Ionizable Lipids and or Cationic Lipids

The scope of ionizable lipids contemplated for use in the present disclosure is not limited to ionizable lipids of Formula (I). In the lipid nanoparticles of the disclosure, the cationic lipid or ionizable lipid may comprise. e.g., one or more of the following: (6Z,9Z,28Z,31Z)-heptatriaconta-6,9,28,31-tetraen-19-yl 4-(dimethylamino)butanoate (DLinMC3DMA), [(4-hydroxybutyl)azanediyl]di(hexane-6,1-diyl) bis(2-hexyldecanoate) (ALC-0315), heptadecan-9-yl 8-{(2-hydroxyethyl)[6-oxo-6-(undecyloxy)hexyl]amino}octanoate (SM-102), 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethylaminopropyl)-[1,3]-dioxolane (D Lin-K-C3-D MA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dili-noleyl-4-dimethylaminomethyl-[1,3]-dioxolane (DLin-KDMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (D Lin-MPZ), 3-(N,N-dilinoleylamino)-1,2-propanediol (D LinAP), 3-(N,N-dioleylamino)-1,2-propanedio (DOAP), 1,2-dilinoleyloxo-3-(2-N,N-dimethylamino)ethoxypropane (D Lin-EG-DMA), N,N-dioleyl-N,N-dimethylammmonium chloride (DODAC), 1,2-dioleyloxy-N,N-dimethylaminopropane (DODMA), 1,2-distearyloxy-N,N-dimethylaminopropane (DSD MA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N, N-trimethylammonium chloride (DOTAP), 3-(N—(N′,N′dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamidoethyl]-N,N-dimethy 1-1-propanaminiumtrifluoroacetate (DOSPA), dioctadecylamidoglycyl spermine (DOGS), 3-dimethylamino-2-(cholest-5-en-3-beta-oxy butan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof. The ionizable lipids are not limited to those recited herein, and can further include ionizable lipids known to those skilled in the art, or described in PCT Application No. PCT/US2020/056255 and/or PCT Application No. PCT/US2020/056252, the disclosures of which are herein incorporated by reference in its entirety.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Application Publication No. US 2011/0256175, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.Cl, DLin-TAP.Cl, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. 20060240554, the disclosure of which is herein incorporated by reference in its entirety for all purposes.

### Non-Cationic Lipid

In the nucleic acid-lipid particles of the present disclosure, the non-cationic lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S—NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (C₁₈), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, l-[8′-(1,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2.000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Lipid Nanoparticles (LNPs)

In one aspect, the present disclosure provides an immune cell targeted lipid nanoparticle (LNP).

In certain embodiments, the LNP comprises at least one ionizable lipid.

In certain embodiments, the LNP comprises at least one neutral lipid.

In certain embodiments, the LNP comprises cholesterol and/or a modified derivative thereof.

In certain embodiments, the LNP comprises at least one polymer conjugated lipid and/or modified derivative thereof, and/or a modified derivative thereof.

In certain embodiments, the LNP comprises a cell targeting domain specific to binding to a surface molecule of a target cell.

In certain embodiments, the cell targeting domain is covalently conjugated to at least one component of the LNP.

In certain, non-limiting, exemplary embodiments, the present disclosure provides a LNP. In certain embodiments, the LNP comprises (a) at least one ionizable lipid. In certain embodiments, the LNP comprises (b) at least one neutral lipid. In certain embodiments, the LNP comprises (c) at least one cholesterol compound and/or modified derivative thereof. In certain embodiments, the LNP comprises (d) at least one polymer conjugated lipid and at least one compound of Formula (II), or a salt, solvate, stereoisomer, or isotopologue thereof.

In certain, non-limiting, exemplary embodiments, the present disclosure provides a LNP. In certain embodiments, the LNP comprises (a) at least one ionizable lipid of Formula (I), or a salt, solvate, stereoisomer, or isotopologue thereof. In certain embodiments, the LNP comprises (b) at least one neutral lipid. In certain embodiments, the LNP comprises (c) at least one cholesterol compound and/or modified derivative thereof. In certain embodiments, the LNP comprises (d) at least one polymer conjugated lipid. In certain embodiments, the LNP comprises (e) at least one cell targeting domain specific to binding a surface molecule of a target cell. In certain embodiments, the cell targeting domain is covalently conjugated to at least one component of the LNP.

In certain, non-limiting, exemplary embodiments, the present disclosure provides a LNP. In certain embodiments, the LNP comprises (a) at least one ionizable lipid of Formula (I), or a salt, solvate, stereoisomer, or isotopologue thereof. In certain embodiments, the LNP comprises (b) at least one neutral lipid. In certain embodiments, the LNP comprises (c) at least one cholesterol compound and/or modified derivative thereof. In certain embodiments, the LNP comprises (d) at least one polymer conjugated lipid and at least one compound of Formula (II), or a salt, solvate, stereoisomer, or isotopologue thereof.

In certain embodiments, the at least one ionizable lipid comprises an ionizable lipid of Formula (I), or a salt, solvate, stereoisomer, or isotopologue thereof.

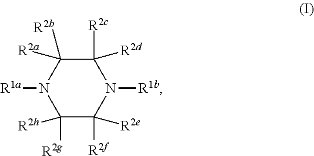

wherein:
R^{1a} and R^{1b} are each independently

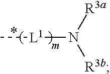

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{1b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;

Each occurrence of L¹ is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of in is independently an integer selected from the group consisting of 1, 2, 3, and 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R², R^{2b}, R^{2c}, R^{1d}, R^{2e}, R^{2f}, R^{2g}, and R^{2h} are H. In certain embodiments, at least six selected from the group consisting of R^{2a}, R^{1b}, R^{2c}, R^{2d}, R^{2e}, R^{2f}, R^{2g}, and R^{1b} are H. In certain embodiments, at least seven selected from the group consisting of R², R^{2b}, R^{2c}R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L¹ is —CH₂—. In certain embodiments, L¹ is —(CH₂)₂—. In certain embodiments, L¹ is —(CH₂)₃—. In certain embodiments, L¹ is —(CH₂)₁₀—. In certain embodiments, L¹ is —(CH₂)₂O—. In certain embodiments, L¹ is —(CH₂)₃O—. In certain embodiments, L¹ is —CH₂CH(OR⁵)CH₂—. In certain embodiments, L¹ is —(CH₂)₂NR^{3c}—. In certain embodiments, L¹ is

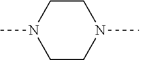

In certain embodiments, L¹ is

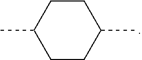

In certain embodiments, L¹ is

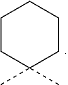

For instances of L which are asymmetric (e.g., —(CH₂)₃O—) it is understood that the present disclosure encompasses both possible orientations (e.g., —(CH₂)₃O— and —O(CH₂)₃—).

In certain embodiments, the ionizable lipid of Formula (I) is:

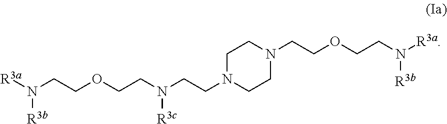

In certain embodiments, the ionizable lipid of Formula (I) is:

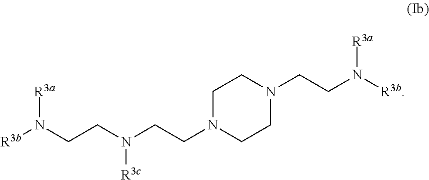

In certain embodiments, the ionizable lipid of Formula (I) is:

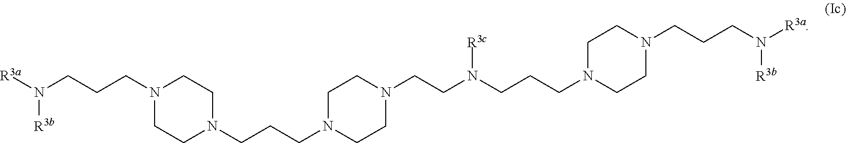

In certain embodiments, the ionizable lipid of Formula (I) is:

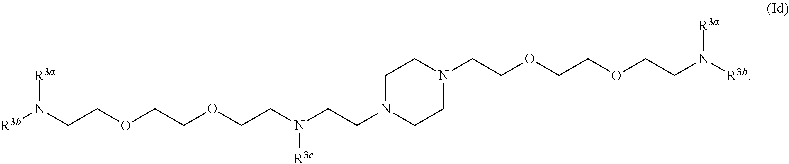

In certain embodiments, the ionizable lipid of Formula (I) is:

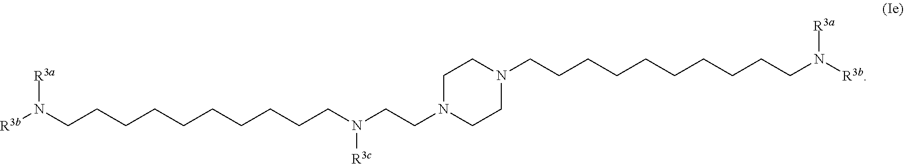

In certain embodiments, the ionizable lipid of Formula (I) is:

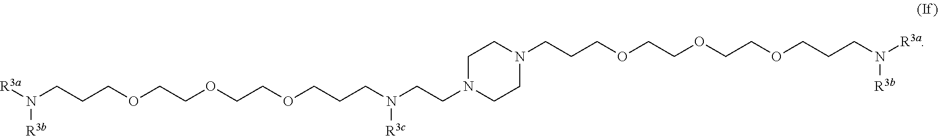

In certain embodiments, the ionizable lipid of Formula (I) is:

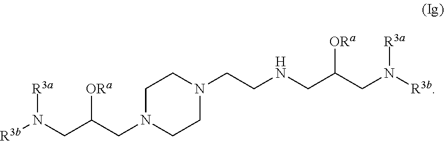

In certain embodiments, the ionizable lipid of Formula (I) is:

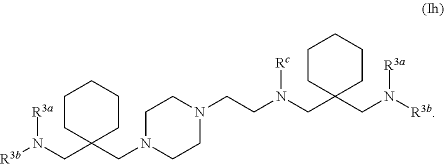

In certain embodiments, the ionizable lipid of Formula (I) is:

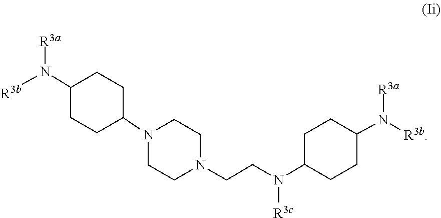

In certain embodiments, R^{3a} is H. In certain embodiments, R^{3a} is —CH₂CH(OH)(optionally substituted C₁-C₂₈ alkyl). In certain embodiments, R^{3a} is —CH₂CH(OH)(optionally substituted C₂-C₂₈ alkenyl). In certain embodiments, R^{3a} is —CH₂CH₂C(═O)O (optionally substituted C₁-C₂₈ alkyl). In certain embodiments, R^{3a} is —CH₂CH₂C(═O)NH (optionally substituted C₁-C₂₈ alkyl). In certain embodiments. R^{3b} is H. In certain embodiments, R^{3b} is —CH₂CH(OH)(optionally substituted C₁-C₂₈ alkyl). In certain embodiments, R^{3b} is —CH₂CH(OH)(optionally substituted C₂-C₂₈ alkenyl). In certain embodiments, R^{3b} is —CH₂CH₂C(═O)O (optionally substituted C₁-C₂₈ alkyl). In certain embodiments, R^{3b} is —CH₂CH₂C(═O)NH (optionally substituted C₁-C₂₈ alkyl). In certain embodiments, R^{3c} is H. In certain embodiments, R^{3c} is —CH₂CH(OH)(optionally substituted C₁-C₂₈ alkyl). In certain embodiments, R^{3c} is —CH₂CH(OH)(optionally substituted C₂-C₂₈ alkenyl). In certain embodiments, R^{3c} is —CH₂CH₂C(═O)O (optionally substituted C₁-C₂₈ alkyl). In certain embodiments, R^{3c} is —CH₂CH₂C(═O)NH (optionally substituted C₁-C₂₈ alkyl).

In certain embodiments, R^{3a} is —CH₂CH(OH)(CH₂)₉CH₃. In certain embodiments. R^{3a} is —CH₂CH(OH)(CH₂)₁₁CH₃. In certain embodiments, R^{3a} is —CH₂CH(OH)(CH₂)₁₃CH₃. In certain embodiments, R^{3b} is —CH₂CH(OH)(CH₂)₉CH₃. In certain embodiments, R^{3b} is —CH₂CH(OH)(CH₂)₁₁CH₃. In certain embodiments, R^{3b} is —CH₂CH(OH)(CH₂)₁₃CH₃. In certain embodiments, R^{3c} is —CH₂CH(OH)(CH₂)₉CH₃. In certain embodiments, R^{3C} is —CH₂CH(OH)(CH₂)₁₁CH₃. In certain embodiments, R³, is —CH₂CH(OH)(CH₂)₁₃CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxv, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the ionizable lipid of Formula (I) is:

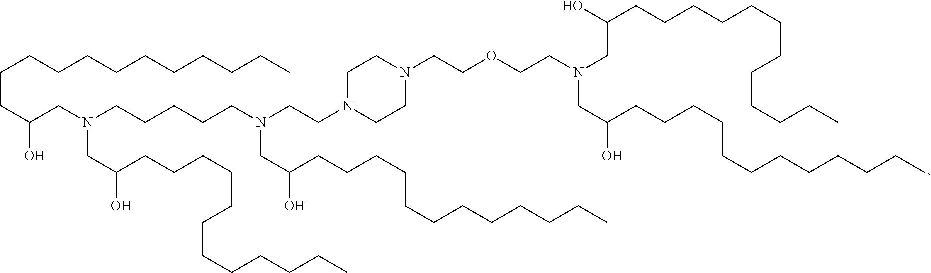 [SMILES: CCCCCCCCCCCCC(O)CN(CCCCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol) (C14-494).

In certain embodiments, the at least one ionizable lipid comprises about 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one ionizable lipid comprises less than about 10, 11, 12, 13, 14, 15, 16, 17, 18, 19, 20, 21, 22, 2324, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the at least one ionizable lipid comprises more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one ionizable lipid comprises about 40 mol % of the LNP. In certain embodiments, the at least one ionizable lipid comprises about 41 mol % of the LNP.

In certain embodiments, the neutral lipid comprises dioleoylphosphatidylethanolamine (DOPE) and distearoylphosphatidylcholine (DSPC). In certain embodiments, the neutral lipid is dioleoylphosphatidylethanolamine (DOPE). In certain embodiments, the neutral lipid is dioleoylphosphatidylethanolamine (DOPE).

In certain embodiments, the at least one neutral lipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the at least one neutral lipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the at least one neutral lipid comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one neutral lipid comprises about 30 mol % of the LNP. In certain embodiments, the LNP comprises about 30 mol % DOPE.

In certain embodiments, the cholesterol and/or modified derivative thereof comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the cholesterol and/or modified derivative thereof comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the cholesterol and/or modified derivative thereof comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the cholesterol and/or modified derivative thereof is cholesterol. In certain embodiments, the cholesterol comprises about 25 mol % of the LNP. In certain embodiments, the cholesterol comprises about 25.6 mol % of the LNP.

In certain embodiments, the at least one polymer conjugated lipid and/or modified derivative thereof comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.2, 5.4, 5.5, 5.6, 5.7, 5.8, 5.9, 6.0, 6.1, 6.2, 6.2, 6.4, 6.5, 6.6, 6.7, 6.8, 6.9, 7.0, 7.1, 7.2, 7.2, 7.4, 7.5, 7.6, 7.7, 7.8, 7.9, 8.0, 8.1, 8.2, 8.2, 8.4, 8.5, 8.6, 8.7, 8.8, 8.9, 9.0, 9.1, 9.2, 9.2, 9.4, 9.5, 9.6, 9.7, 9.8, 9.9, 10.0, 10.1, 10.2, 10.2, 10.4, 10.5, 10.6, 10.7, 10.8, 10.9, 11.0, 11.1, 11.2, 11.2, 11.4, 11.5, 11.6, 11.7, 11.8, 11.9, 12.0, 12.1, 12.2, 12.3, 12.4, or about 12.5 mol % of the LNP.

In certain embodiments, the at least one polymer conjugated lipid and/or modified derivative thereof comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.2, 5.4, 5.5, 5.6, 5.7, 5.8, 5.9, 6.0, 6.1, 6.2, 6.2, 6.4, 6.5, 6.6, 6.7, 6.8, 6.9, 7.0, 7.1, 7.2, 7.2, 7.4, 7.5, 7.6, 7.7, 7.8, 7.9, 8.0, 8.1, 8.2, 8.2, 8.4, 8.5, 8.6, 8.7, 8.8, 8.9, 9.0, 9.1, 9.2, 9.2, 9.4, 9.5, 9.6, 9.7, 9.8, 9.9, 10.0, 10.1, 10.2, 10.2, 10.4, 10.5, 10.6, 10.7, 10.8, 10.9, 11.0, 11.1, 11.2, 11.2, 11.4, 11.5, 11.6, 11.7, 11.8, 11.9, 12.0, 12.1, 12.2, 12.3, 12.4, or about 12.5 mol % of the LNP.

In certain embodiments, the at least one polymer conjugated lipid and/or modified derivative thereof comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.2, 5.4, 5.5, 5.6, 5.7, 5.8, 5.9, 6.0, 6.1, 6.2, 6.2, 6.4, 6.5, 6.6, 6.7, 6.8, 6.9, 7.0, 7.1, 7.2, 7.2, 7.4, 7.5, 7.6, 7.7, 7.8, 7.9, 8.0, 8.1, 8.2, 8.2, 8.4, 8.5, 8.6, 8.7, 8.8, 8.9, 9.0, 9.1, 9.2, 9.2, 9.4, 9.5, 9.6, 9.7, 9.8, 9.9, 10.0, 10.1, 10.2, 10.2, 10.4, 10.5, 10.6, 10.7, 10.8, 10.9, 11.0, 11.1, 11.2, 11.2, 11.4, 11.5, 11.6, 11.7, 11.8, 11.9, 12.0, 12.1, 12.2, 12.3, 12.4, or about 12.5 mol % of the LNP.

In certain embodiments, the at least one polymer conjugated lipid and/or modified derivative thereof comprises about 2.5 mol % of the LNP.

In certain embodiments, the at least one polymer conjugated lipid and/or modified derivative thereof comprises a polyethylene glycol (PEG) conjugated lipid and/or modified derivative thereof. In certain embodiments, the at least one polyethylene glycol (PEG) conjugated lipid and/or modified derivative thereof comprises C14-PEG2000. In certain embodiments, C14-PEG2000 comprises (1,2-dimyristoyl-sn-glycero-3-phosphoethanolamine-N-[methoxy(polyethylene glycol)-2000]:

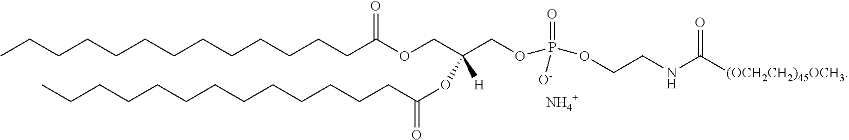

In certain embodiments, the maleimide moiety is covalently linked to the PEG chain of the PEG-conjugated lipid. A non-limiting example of covalent linkage to PEG chain of the PEG-conjugated lipid includes:

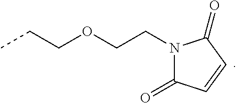

Alternatively, the covalent linkage to the PEG chain may comprise any of a number of bonds, including but not limited to an amide bond.

In certain embodiments, the surface molecule of a target cell is a surface antigen of a CD4+ T cell. In certain embodiments, the surface molecule of a target cell is a surface antigen of a CD8+ T cell.

In certain embodiments, the cell targeting domain specific to binding a surface molecule of a target cell is at least one selected from the group consisting of an antibody against CD3 (αCD3) and an antibody against CD28 (αCD28), or a fragment thereof.

In certain embodiments, the component to which the cell targeting domain is conjugated is the polymer conjugated lipid and/or modified derivative thereof and/or modified derivative thereof.

In certain embodiments, the targeting domain is covalently conjugated to the polymer conjugated lipid and/or modified derivative thereof and/or modified derivative thereof.

In certain embodiments, the covalent conjugation comprises a covalent bond forming reaction selected from the group consisting of a [1,4]-conjugate addition (i.e., Michael addition), [4+2] cycloaddition. [3+2] dipolar cycloaddition, nucleophilic addition, transition metal-catalyzed cross-coupling reaction, carbonyl condensation reaction, and reductive amination.

In certain embodiments, the covalent conjugation reaction comprises a [1,4]-conjugate addition reaction (i.e., Michael addition).

In certain embodiments, the [1,4]-conjugate addition occurs between a PEG-polyethylene glycol (PEG) conjugated lipid and/or modified derivative thereof which is further conjugated to a maleimide moiety and a cysteine thiol of a polypeptide.

In certain embodiments, the cystine thiol of the polypeptide is derived from a reduced disulfide bridge of a polypeptide selected from the group consisting of an antibody against CD3 and an antibody against CD28, or a fragment thereof.

Alternative non-limiting examples of complementary functional groups for conjugation of the lipid-conjugate and targeting domain include: (a) a nucleophile and electrophile (e.g., SN₁ or SN₂ reaction of an hydroxyl and benzyl chloride or an amine and a carboxylic acid or derivative thereof); (b) an azide and an alkyne (i.e., [3+2] cycloaddition or “click” reaction); and (c) a diene and a dienophile (e.g., substituted butadiene and substituted maleimide) via a Diels-Alder [4+2] cycloaddition, inter alia.

It is appreciated that any of a number covalent bond forming reactions (e.g., SN₂, condensation, Diels-Alder reaction (i.e., [4+2] cycloaddition), [3+2]dipolar cycloaddition, and transition metal catalyzed cross-coupling, inter alia) may be employed to prepare the conjugated compositions of the present disclosure. It is understood that, given a particular bond forming reaction (e.g., SN₂ reaction), one skilled in the art would readily recognize the requisite functional groups suitable for each component (i.e., conjugated lipid and targeting domain) necessary to achieve conjugation. Additionally, one skilled in the art of organic synthesis would be apprised of the necessary additional reagents and/or catalyst necessary to achieve covalent bond formation.

In certain embodiments, the LNP has a molar ratio of PEG-polyethylene glycol (PEG) conjugated lipid and/or modified derivative thereof and PEG-polyethylene glycol (PEG) conjugated lipid and/or modified derivative thereof further conjugated to a maleimide moiety selected from the group consisting of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10.

In certain embodiments, the LNP has a molar ratio of PEG-polyethylene glycol (PEG) conjugated lipid and/or modified derivative thereof and PEG-polyethylene glycol (PEG) conjugated lipid and/or modified derivative thereof further conjugated to a maleimide moiety of about 5:1.

In certain embodiments, the LNP has a molar ratio of polymer conjugated lipid and modified derivative of the conjugated lipid further conjugated to a maleimide moiety selected from the group consisting of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10.

In certain embodiments, the modified derivative of the polymer conjugated lipid is a compound of Formula (II), or a salt, solvate, stereoisomer, or isotopologue thereof:

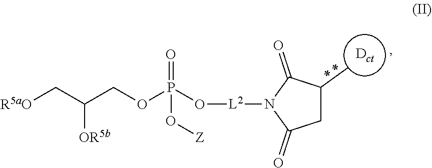

wherein:
R^{5a} and R^{5b} are each independently selected form the group consisting of —C(═O)(optionally substituted C₁-C₂₈ alkyl), —C(═O)(optionally substituted C₂-C₂₈ alkenyl). —C(═O)(optionally substituted C₂-C₂₈ alkynyl), optionally substituted C₁-C₂₈ alkyl, optionally substituted C₂-C₂₈ alkenyl, and optionally substituted C₂-C₂₈ alkynyl;
Z is a monovalent cation;
L² comprises n units of

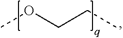

[symbol]

units of

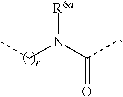

and p units of

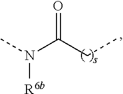

wherein each ---- in L² is a C—O or C—N bond,
D_{ct} is a cell targeting domain comprising an antibody against CD3 or CD28, wherein [symbol] is C—S bond; R^{6a} and R^{6b} bare each independently selected from the group consisting of H and C₁-C₆ alkyl;
n, [symbol] , and p are each independently 1, 2, 3, 4, or 5;
q is an integer ranging from 1 to 100; and
r and s are each independently an integer ranging from 1 to 10.

In certain embodiments, R^{5a} is C(═O)(C₅-C₂₀ alkyl). In certain embodiments, R^{5a} is C(═O)(CH₂)₁₆CH₃. In certain embodiments, R^{5b} is C(═O)(C₅-C₂₀ alkyl). In certain embodiments, R^{5b} is C(═O)(CH₂)₁₆CH₃.

In certain embodiments, Z is NH₄⁺.

In certain embodiments, L² is

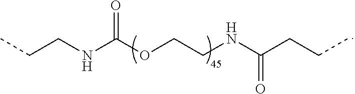

In certain embodiments, the compound of Formula (II) is:

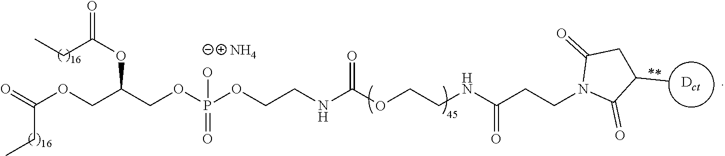

In certain embodiments, Dt comprises at least one of an antibody of CD3 (αCD3) and an antibody of CD28 (αCD28). In certain embodiments, D_{ct} comprises an antibody of CD3 (αCD3). In certain embodiments, D_{ct} comprises an antibody of CD28 (αCD28). In certain embodiments. De comprises an antibody of CD3 (αCD3) and an antibody of CD28 (αCD28). In certain embodiments, D_{ct} comprises an antibody of CD3 ((CD3) only. In certain embodiments, De comprises an antibody of CD28 (αCD28) only. In certain embodiments, the antibody of CD3 and the antibody of CD28 have a ratio ranging from about 100:1, 90:10, 80:20, 70:30, 60:40, 50:50, 40:60, 30:70, 20:80, 10:90, or about 1:100 ((CD3:αCD28).

In certain embodiments, (d) comprises the polymer conjugated lipid and the compound of formula (II), wherein the polymer conjugated lipid and the compound of formula (II) have a molar ratio of about 4.9:0.1, 4.8:0.2, 4.7:0.3, 4.6:0.4, 4.5:0.5, 4.4:0.6, 4.3:0.7, 4.2:0.8, 4.1:0.9, 4.0:1.0, 3.9:1.1, 3.8:1.2, 3.7:1.3, 3.6:1.4, 3.5:1.5, 3.4:1.6, 3.3:1.7, 3.2:1.8, 3.1:1.9, 3.0:2.0, 2.9:2.1, 2.8:2.2, 2.7:2.3, 2.6:2.4, 2.5:2.5, 2.4:2.6, 2.3:2.7, 2.2:2.8, 2.1:2.9, 2.0:3.0, 1.9:3.1, 1.8:3.2, 1.7:3.3, 1.6:3.4, 1.5:3.5, 1.4:3.6, 1.3:3.7, 1.2:3.8, 1.1:3.9, 1.0:4.0, 0.9:4.1, 0.8:4.2, 0.7:4.3, 0.6:4.4, 0.5:4.5, 0.4:4.6, 0.3:4.7, 0.2:4.8, or about 0.1:4.9.

In certain embodiments, the LNP has a molar ratio of (a):(b):(c):(d) of about 40:30:25:2.5. In certain embodiments, the LNP has a molar ratio of (a):(b):(c):(d) of about 41:30.8:25.6:2.5. In certain embodiments, (d) comprises the polymer conjugated lipid and the compound of formula (II) having a ratio of about 2.1:0.4.

In certain embodiments, the LNP further comprises at least one cargo selected from the group consisting of a nucleic acid molecule and a therapeutic agent.

In certain embodiments, the therapeutic agent is at least one selected from the group consisting of a small molecule, a protein, and an antibody.

In certain embodiments, the LNP comprises a nucleic acid molecule.

In certain embodiments, the nucleic acid molecule is a DNA molecule or an RNA molecule.

In certain embodiments, the nucleic acid molecule is selected from the group consisting of cDNA, mRNA, miRNA, siRNA, modified RNA, antagomir, antisense molecule, and a targeted nucleic acid, or any combination thereof.

In certain embodiments, the nucleic acid molecule encodes a chimeric antigen receptor (CAR).

In certain embodiments, the CAR is specific for binding to a surface antigen of a pathogenic cell or a tumor cell.

In certain embodiments, the surface antigen is selected from the group consisting of CD4, CD8, CD1, CD2, CD3, CD5, CD7, CD16, CD19, CD20, CD22, CD25, CD26, CD27, CD28, CD30, CD33, CD38, CD39, CD40L, CD44, CD45, CD62L, CD69, CD73, CD80, CD83, CD86, CD95, CD103, CD119, CD123, CD126, CD150, CD153, CD154, CD161, CD183, CD223, CD254, CD275, CD45RA, CXCR3, CXCR5. FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, CCR7, k light chain, ROR1, ErbB2, ErbB3, ErbB4, EGFR vIII, carcinoembryonic antigen, EGP2, EGP40, mesothelin, TAG72, PSMA, NKG2D ligands. B7-H6, IL13R-α2. MUC1, VEGF-A, Tem8, FAP, EphA2, HER2, MUC16, CA9, GD2, GD3, HMW-MAA, CD171, Lewis Y, G250/CALX, HLA-AI MAGE A1, HAL-A2 NY-ESO-1, PSC1, folate receptor-α, 8H9, NCAM, VEGF, 5T4, Fetal AchR, NKG2D ligands, TEM1, and TEM8.

In certain embodiments, the nucleic acid molecule encodes mRNA. In certain embodiments, the nucleic acid molecule encodes sgRNA. In certain embodiments, the nucleic acid molecule encodes mRNA and sgRNA. In certain embodiments, the mRNA encodes a therapeutic protein. In certain embodiments, the therapeutic protein is a CRISPR-associated protein. In certain embodiments, the CRISPR-associated protein is CRISPR-associated protein 9 (Cas9).

In certain embodiments, the therapeutic agent is a CRISPR-associated protein. In certain embodiments, the CRISPR-associated protein is CRISPR-associated protein 9 (Cas9).

### Cell Targeting Domain

In various embodiments of the invention, the LNP of the invention is conjugated to a targeting domain specific for binding to a receptor of a target cell.

In certain embodiments, the target cell is a stem cell. Exemplary stem cells that can be targeted by the compositions of the invention include, but are not limited to, hematopoietic stem cells and stem cells related to hematopoietic stem cells (e.g., myeloid stem cells and lymphoid stem cells.)

In certain embodiments, the target cell is a peripheral blood mononuclear cell (PBMC).

In one cell the target cell is an immune cell. Exemplary immune cells that can be targeted according by the compositions of the invention include, but are not limited to, T cells, B cells, NK cells, antigen-presenting cells, dendritic cells, macrophages, monocytes, neutrophils, eosinophils, and basophils. In certain embodiments, the immune cell is a T cell. In some embodiments. T cells that can be targeted using the compositions of the invention can be CD4+ or CD8+ and can include, but are not limited to, T helper cells (CD4+), cytotoxic T cells (also referred to as cytotoxic T lymphocytes, CTL; CD8− T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th17 cells, Th22 cells, Tfh (follicular helper) cells, T regulatory cells, natural killer T cells, mucosal associated invariant T cells (MAIT), and γδ T cells. Major T cell subtypes include T_{N} (naive). T_{SCM} (stem cell memory), T_{CM} (central memory), T_{TM} (Transitional Memory), T_{EM} (Effector memory), and T_{TE} (Terminal Effector), TCR-transgenic T cells, T-cells redirected for universal cytokine-mediated killing (TRUCK), Tumor infiltrating T cells (TIL), CAR-T cells or any T cell that can be used for treating a disease or disorder.

In certain embodiments, the T cells of the invention are immunostimulatory cells, i.e., cells that mediate an immune response. Exemplary T cells that are immunostimulatory include, but are not limited to, T helper cells (CD4+), cytotoxic T cells (also referred to as cytotoxic T lymphocytes, CTL; CD8+ T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th17 cells, Th22 cells, Tfh (follicular helper) cells, natural killer T cells, mucosal associated invariant T cells (MAIT), and γδ T cells.

In certain embodiments, the T cell targeting domain binds to CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA. CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, or CCR7.

In certain embodiments, present invention relates to compositions comprising a combination of delivery vehicles conjugated to immune cell targeting domains for targeting multiple immune cells. In certain embodiments, the combination comprises two or more immune cell targeted delivery vehicles, targeting two or more immune cell antigens. In certain embodiments, the two or more immune cell antigens are selected from CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3. TLR4, TLR6, NKG2D, CCR, CCR1, CCR2, CCR4, CCR6, and CCR7. In certain embodiments, the combination comprises two or more T cell targeted delivery vehicles, targeting a surface antigen of a CD4+ T cell and a surface antigen of a CD8+ T cell. In certain embodiments, the combination comprises two or more T cell targeted delivery vehicles, targeting CD4 and CD8.

In certain embodiments, the targeting domain is conjugated to the LNP of the invention. Exemplary methods of conjugation can include, but are not limited to, covalent bonds, electrostatic interactions, and hydrophobic (“van der Waals”) interactions. In certain embodiments, the conjugation is a reversible conjugation, such that the delivery vehicle can be disassociated from the targeting domain upon exposure to certain conditions or chemical agents. In some embodiments, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In certain embodiments, the activated polymer conjugated lipid comprises a first coupling group capable of reacting with a second coupling group. In certain embodiments, the activated polymer conjugated lipid is an activated pegylated lipid. In certain embodiments, the first coupling group is bound to the lipid portion of the pegylated lipid. In some embodiments, the first coupling group is bound to the polyethylene glycol portion of the pegylated lipid. In certain embodiments, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In certain embodiments, the first coupling group is a maleimide.

In certain embodiments, the second coupling group is a sulfhydryl group. The sulfhydryl group can be installed on the targeting domain using any method known to those of skill in the art. In certain embodiments, the sulfhydryl group is present on a free cysteine residue. In certain embodiments, the sulfhydryl group is revealed via reduction of a disulfide on the targeting domain, such as through reaction with 2-mercaptoethylamine. In certain embodiments, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone.

In certain embodiments, the targeting domain is conjugated to the LNP using maleimide conjugation.

### Targeting Domain

In certain embodiments, the composition comprises a targeting domain that directs the delivery vehicle to a target immune cell. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In various embodiments, the targeting domain binds to a cell surface molecule of a cell of interest. For example, in various embodiments, the targeting domain binds to a cell surface molecule of an endothelial cell, a stem cell, or an immune cell.

### Peptides

In certain embodiments, the targeting domain of the invention comprises a peptide. In certain embodiments, the peptide targeting domain specifically binds to a target of interest.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention. (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Nucleic Acids

In certain embodiments, the targeting domain of the invention comprises an isolated nucleic acid, including for example a DNA oligonucleotide and a RNA oligonucleotide. In certain embodiments, the nucleic acid targeting domain specifically binds to a target of interest. For example, In certain embodiments, the nucleic acid comprises a nucleotide sequence that specifically binds to a target of interest.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

### Antibodies

In certain embodiments, the targeting domain of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### LNP Cargo

### Anti-Cancer Agents

In one embodiment, the at least one additional agent is an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but are not limited to, chemotherapeutic agents, antiproliferative agents, anti-tumor agents, checkpoint inhibitors, and anti-angiogenic agents. For example, in one embodiment, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ. BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomvcin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkvlating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine: busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin: dezaguanine: dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine: fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide: ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to; 20-epi-1,25 dihydroxyvitamin D3; 5-ethvnyluracil; abiraterone; aclarubicin; acylfulvene; adecvpenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase;
metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tvrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs, biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acids

In certain embodiments, the invention includes an ionizable LNP molecule formulated for targeted in vivo T cell delivery comprising or encapsulating one or more nucleic acid molecule. In certain embodiments, the nucleic acid molecule is a mRNA molecule. In certain embodiments, the mRNA molecule encodes a CAR. In certain embodiments, the nucleoside-modified mRNA molecule encodes a CAR. In certain embodiments, the invention includes a nucleoside-modified mRNA molecule encoding an adjuvant.

The nucleotide sequences encoding an CAR, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode an antigen or antigen binding molecule or adjuvant of interest.

Further, the scope of the invention includes nucleotide sequences that encode amino acid sequences that are substantially homologous to the amino acid sequences recited herein and preserve the immunogenic function of the original amino acid sequence.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In certain embodiments, the invention relates to a construct, comprising a nucleotide sequence encoding a CAR. In certain embodiments, the construct comprises a plurality of nucleotide sequences encoding a plurality of antigens. For example, in certain embodiments, the construct encodes 1 or more, 2 or more, 5 or more, 10 or more, 15 or more, or 20 or more antigens. In certain embodiments, the invention relates to a construct, comprising a nucleotide sequence encoding an adjuvant. In certain embodiments, the construct comprises a first nucleotide sequence encoding a CAR and a second nucleotide sequence encoding an adjuvant.

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding a CAR; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Nucleoside-Modified RNA

In certain embodiments, the composition comprises a nucleoside-modified RNA. In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy. In certain embodiments, an immune cell comprising an expressing a mRNA molecule encoding the CAR is directed to a cell of interest expressing an antigen that is specifically bound by the CAR.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005. Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010. Nucleic Acids Res 38:5884-5892). A preparative HPLC purification procedure has been established that was critical to obtain pseudouridine-containing RNA that has superior translational potential and no innate immunogenicity (Karikó et al., 2011, Nucleic Acids Research 39:e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In certain embodiments, the nucleoside-modified RNA of the invention is IVT RNA. For example, in certain embodiments, the nucleoside-modified RNA is synthesized by T7 phage RNA polymerase. In some embodiments, the nucleoside-modified mRNA is synthesized by SP6 phage RNA polymerase. In some embodiments, the nucleoside-modified RNA is synthesized by T3 phage RNA polymerase.

In certain embodiments, the modified nucleoside is m¹acp³Ψ (1-methyl-3-(3-amino-3-carboxypropyl) pseudouridine. In some embodiments, the modified nucleoside is m¹Ψ (1-methylpseudouridine). In some embodiments, the modified nucleoside is Ψm (2′-O-methylpseudouridine. In some embodiments, the modified nucleoside is m⁵D (5-methyldihydrouridine). In some embodiments, the modified nucleoside is m³Ψ (3-methylpseudouridine). In some embodiments, the modified nucleoside is a pseudouridine moiety that is not further modified. In some embodiments, the modified nucleoside is a monophosphate, diphosphate, or triphosphate of any of the above pseudouridines. In some embodiments, the modified nucleoside is any other pseudouridine-like nucleoside known in the art.

In some embodiments, the nucleoside that is modified in the nucleoside-modified RNA the present invention is uridine (U). In some embodiments, the modified nucleoside is cytidine (C). In some embodiments, the modified nucleoside is adenosine (A). In another embodiment the modified nucleoside is guanosine (G).

In some embodiments, the modified nucleoside of the present invention is m⁵C (5-methylcytidine). In some embodiments, the modified nucleoside is m⁵U (5-methyluridine). In some embodiments, the modified nucleoside is m⁶A (N⁶-methyladenosine). In some embodiments, the modified nucleoside is s²U (2-thiouridine). In some embodiments, the modified nucleoside is Ψ (pseudouridine). In some embodiments, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms^{2h} n⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²2G (N²,N²-dimethylguanosine); m²Gm (N²,2′-O-dimethylguanosine); m²2Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxy carbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶2A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶2Am (N⁶,N⁶,O-2′-trimethyladenosine); m²° 7G (N²,7-dimethylguanosine); m²,2° ⁷G (N²,N²,7-trimethylguanosine); m³Um (3,2′-O-dimethyluridine); m⁵D (5-methyldihyrouridine); f⁵Cm (5-formyl-2′-O-methylcytidine); miGm (1,2′-O-dimethylguanosine); miAm (1,2′-O-dimethyladenosine); τm⁵U (5-taurinomethyluridine); τm⁵s²U (5-taurinomethyl-2-thiouridine)); imG-14 (4-demethylwyosine); imG2 (isowvosine); or ac⁶A (N⁶-acetyladenosine).

In some embodiments, a nucleoside-modified RNA of the present invention comprises a combination of 2 or more of the above modifications. In some embodiments, the nucleoside-modified RNA comprises a combination of 3 or more of the above modifications. In some embodiments, the nucleoside-modified RNA comprises a combination of more than 3 of the above modifications.

In some embodiments, between 0.10% and 100% of the residues in the nucleoside-modified of the present invention are modified (e.g. either by the presence of pseudouridine or a modified nucleoside base). In some embodiments, 0.1% of the residues are modified. In some embodiments, the fraction of modified residues is 0.2%. In some embodiments, the fraction is 0.3%. In some embodiments, the fraction is 0.4%. In some embodiments, the fraction is 0.5%. In some embodiments, the fraction is 0.6%. In some embodiments, the fraction is 0.8%. In some embodiments, the fraction is 1%. In some embodiments, the fraction is 1.5%. In some embodiments, the fraction is 2%. In some embodiments, the fraction is 2.5%. In some embodiments, the fraction is 3%. In some embodiments, the fraction is 4%. In some embodiments, the fraction is 5%. In some embodiments, the fraction is 6%. In some embodiments, the fraction is 8%. In some embodiments, the fraction is 10%. In some embodiments, the fraction is 12%. In some embodiments, the fraction is 14%. In some embodiments, the fraction is 16%. In some embodiments, the fraction is 18%. In some embodiments, the fraction is 20%. In some embodiments, the fraction is 25%. In some embodiments, the fraction is 30%. In some embodiments, the fraction is 35%. In some embodiments, the fraction is 40%. In some embodiments, the fraction is 45%. In some embodiments, the fraction is 50%. In some embodiments, the fraction is 60%. In some embodiments, the fraction is 70%. In some embodiments, the fraction is 80%. In some embodiments, the fraction is 90%. In some embodiments, the fraction is 100%.

In some embodiments, the fraction is less than 5%. In some embodiments, the fraction is less than 3%. In some embodiments, the fraction is less than 1%. In some embodiments, the fraction is less than 2%. In some embodiments, the fraction is less than 4%. In some embodiments, the fraction is less than 6%. In some embodiments, the fraction is less than 8%. In some embodiments, the fraction is less than 10%. In some embodiments, the fraction is less than 12%. In some embodiments, the fraction is less than 15%. In some embodiments, the fraction is less than 20%. In some embodiments, the fraction is less than 30%. In some embodiments, the fraction is less than 40%. In some embodiments, the fraction is less than 50%. In some embodiments, the fraction is less than 60%. In some embodiments, the fraction is less than 70%.

In some embodiments, 0.1% of the residues of a given nucleoside (i.e., uridine, cytidine, guanosine, or adenosine) are modified. In some embodiments, the fraction of the given nucleotide that is modified is 0.2%. In some embodiments, the fraction is 0.3%. In some embodiments, the fraction is 0.4%. In some embodiments, the fraction is 0.5%. In some embodiments, the fraction is 0.6%. In some embodiments, the fraction is 0.8%. In some embodiments, the fraction is 1%. In some embodiments, the fraction is 1.5%. In some embodiments, the fraction is 2%. In some embodiments, the fraction is 2.5%. In some embodiments, the fraction is 3%. In some embodiments, the fraction is 4%. In some embodiments, the fraction is 5%. In some embodiments, the fraction is 6%. In some embodiments, the fraction is 8%. In some embodiments, the fraction is 10%. In some embodiments, the fraction is 12%. In some embodiments, the fraction is 14%. In some embodiments, the fraction is 16%. In some embodiments, the fraction is 18%. In some embodiments, the fraction is 20%. In some embodiments, the fraction is 25%. In some embodiments, the fraction is 30%. In some embodiments, the fraction is 35%. In some embodiments, the fraction is 40%. In some embodiments, the fraction is 45%. In some embodiments, the fraction is 50%. In some embodiments, the fraction is 60%. In some embodiments, the fraction is 70%. In some embodiments, the fraction is 80%. In some embodiments, the fraction is 90%. In some embodiments, the fraction is 100%.

In some embodiments, the fraction of the given nucleotide that is modified is less than 8%. In some embodiments, the fraction is less than 10%. In some embodiments, the fraction is less than 5%. In some embodiments, the fraction is less than 3%. In some embodiments, the fraction is less than 1%. In some embodiments, the fraction is less than 2%. In some embodiments, the fraction is less than 4%. In some embodiments, the fraction is less than 6%. In some embodiments, the fraction is less than 12%. In some embodiments, the fraction is less than 15%. In some embodiments, the fraction is less than 20%. In some embodiments, the fraction is less than 30%. In some embodiments, the fraction is less than 40%. In some embodiments, the fraction is less than 50%. In some embodiments, the fraction is less than 60%. In some embodiments, the fraction is less than 70%.

In some embodiments, a nucleoside-modified RNA of the present invention is translated in the cell more efficiently than an unmodified RNA molecule with the same sequence. In some embodiments, the nucleoside-modified RNA exhibits enhanced ability to be translated by a target cell. In some embodiments, translation is enhanced by a factor of 2-fold relative to its unmodified counterpart. In some embodiments, translation is enhanced by a 3-fold factor. In some embodiments, translation is enhanced by a 5-fold factor. In some embodiments, translation is enhanced by a 7-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the nucleoside-modified antigen-encoding RNA of the present invention induces significantly more adaptive immune response than an unmodified in vitro-synthesized RNA molecule with the same sequence. In some embodiments, the modified RNA molecule exhibits an adaptive immune response that is 2-fold greater than its unmodified counterpart. In some embodiments, the adaptive immune response is increased by a 3-fold factor. In another embodiment the adaptive immune response is increased by a 5-fold factor. In some embodiments, the adaptive immune response is increased by a 7-fold factor. In some embodiments, the adaptive immune response is increased by a 10-fold factor. In some embodiments, the adaptive immune response is increased by a 15-fold factor. In another embodiment the adaptive immune response is increased by a 20-fold factor. In some embodiments, the adaptive immune response is increased by a 50-fold factor. In some embodiments, the adaptive immune response is increased by a 100-fold factor. In some embodiments, the adaptive immune response is increased by a 200-fold factor. In some embodiments, the adaptive immune response is increased by a 500-fold factor. In some embodiments, the adaptive immune response is increased by a 1000-fold factor. In some embodiments, the adaptive immune response is increased by a 2000-fold factor. In some embodiments, the adaptive immune response is increased by another fold difference.

In some embodiments, “induces significantly more adaptive immune response” refers to a detectable increase in an adaptive immune response. In some embodiments, the term refers to a fold increase in the adaptive immune response (e.g., 1 of the fold increases enumerated above). In some embodiments, the term refers to an increase such that the nucleoside-modified RNA can be administered at a lower dose or frequency than an unmodified RNA molecule with the same species while still inducing an effective adaptive immune response. In some embodiments, the increase is such that the nucleoside-modified RNA can be administered using a single dose to induce an effective adaptive immune response.

In some embodiments, the nucleoside-modified RNA of the present invention exhibits significantly less innate immunogenicity than an unmodified in vitro-synthesized RNA molecule with the same sequence. In some embodiments, the modified RNA molecule exhibits an innate immune response that is 2-fold less than its unmodified counterpart. In some embodiments, innate immunogenicity is reduced by a 3-fold factor. In some embodiments, innate immunogenicity is reduced by a 5-fold factor. In some embodiments, innate immunogenicity is reduced by a 7-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor. In some embodiments, innate immunogenicity is reduced by another fold difference.

In some embodiments. “exhibits significantly less innate immunogenicity” refers to a detectable decrease in innate immunogenicity. In some embodiments, the term refers to a fold decrease in innate immunogenicity (e.g., 1 of the fold decreases enumerated above). In some embodiments, the term refers to a decrease such that an effective amount of the nucleoside-modified RNA can be administered without triggering a detectable innate immune response. In some embodiments, the term refers to a decrease such that the nucleoside-modified RNA can be repeatedly administered without eliciting an innate immune response sufficient to detectably reduce production of the recombinant protein. In some embodiments, the decrease is such that the nucleoside-modified RNA can be repeatedly administered without eliciting an innate immune response sufficient to eliminate detectable production of the recombinant protein.

### Polypeptide Therapeutic Agents

In other related aspects, the therapeutic agent includes an isolated peptide that modulates a target. For example, In certain embodiments, the peptide of the invention inhibits or activates a target directly by binding to the target thereby modulating the normal functional activity of the target. In certain embodiments, the peptide of the invention modulates the target by competing with endogenous proteins. In certain embodiments, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### CAR Agents

In certain embodiments, the mRNA molecule of the invention encodes a chimeric antigen receptor (CAR). In certain embodiments, the CAR comprises an antigen binding domain. In certain embodiments, the antigen binding domain is a targeting domain, wherein the targeting domain directs the T cell expressing the CAR to a specific cell or tissue of interest. For example, In certain embodiments, the targeting domain comprises an antibody, antibody fragment, or peptide that specifically binds to an expressed on a pathogenic organism or a tumor cell thereby directing the T cell expressing the CAR to a cell or tissue expressing the antigen.

In certain embodiments, the invention relates to an immune cell targeted LNP comprising an agent, wherein the agent comprises a nucleic acid sequence encoding a chimeric antigen receptor (CAR). In certain embodiments, agent comprises an mRNA molecule encoding a CAR. In certain embodiments, the agent comprises a modified nucleoside mRNA molecule encoding a CAR.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically bome by two separate complexes, the TCR heterodimer and the CD3 complex. “Second generation” CARs include an intracellular domain from various co-stimulatory molecules, for example, CD28, 4-11313, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is an scFv specific for binding to a surface antigen of a target cell of interest (e.g., a pathogen or tumor cell.)

### Combinations

In certain embodiments, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Antigens

The present invention provides a composition that induces an immune response in a subject. In certain embodiments, the composition comprises an immune cell targeted LNP comprising a nucleic acid molecule encoding a chimeric antigen receptor CAR specific for an antigen.

In certain embodiments, the antigen comprises a polypeptide or peptide associated with a pathogen or tumor cell, such that the ex vivo modified immune cell expressing the CAR is then targeted to the antigen, inducing an immune response against the antigen, and therefore the pathogen or tumor cell.

In certain embodiments, the antigen, recognized by the CAR encoded by the nucleic acid molecule, comprises a protein, peptide, a fragment thereof, or a variant thereof, or a combination thereof from any number of organisms, for example, a virus, a parasite, a bacterium, a fungus, or a mammal.

In certain embodiments, the antigen comprises a tumor-specific antigen or tumor-associated antigen, such that the immune cell expressing the CAR is directed to a tumor cell expressing the antigen.

### Viral Antigens

In certain embodiments, the antigen comprises a viral antigen, or fragment thereof, or variant thereof. In certain embodiments, the viral antigen is from a virus from one of the following families: Adenoviridae, Arenaviridae, Bunyaviridae, Caliciviridae, Coronaviridae, Filoviridae, Hepadnaviridae. Herpesviridae, Orthomyxoviridae. Papovaviridae, Paramyxoviridae, Parvoviridae, Picornaviridae, Poxviridae, Reoviridae, Retroviridae, Rhabdoviridae, or Togaviridae. In certain embodiments, the viral antigen is from papilloma viruses, for example, human papillomoa virus (HPV), human immunodeficiency virus (HIV), polio virus, hepatitis B virus, hepatitis C virus, smallpox virus (Variolamajor and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), Califomia encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, severe acute respiratory syndrome (SARS) virus, severe acute respiratory syndrome coronavirus 2 (SARS-CoV-2) or a cancer causing virus.

### Parasite Antigens

In certain embodiments, the antigen comprises a parasite antigen or fragment or variant thereof. In certain embodiments, the parasite is a protozoa, helminth, or ectoparasite. In certain embodiments, the helminth (i.e., worm) is a flatworm (e.g., flukes and tapeworms). a thomy-headed worm, or a round worm (e.g., pinworms). In certain embodiments, the ectoparasite is lice, fleas, ticks, and mites.

In certain embodiments, the parasite is any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis. Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

In certain embodiments, the parasite is Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma. Strongyloides stercoralis, Mite, Tapeworm, Toxoplasma gondii, Trypanosoma, Whipworm, or Wuchereria bancrofti.

### Bacterial Antigens

In certain embodiments, the antigen comprises a bacterial antigen or fragment or variant thereof. In certain embodiments, the bacterium is from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

In certain embodiments, the bacterium is a gram positive bacterium or a gram negative bacterium. In certain embodiments, the bacterium is an aerobic bacterium or an anaerobic bacterium. In certain embodiments, the bacterium is an autotrophic bacterium or a heterotrophic bacterium. In certain embodiments, the bacterium is a mesophile, a neutrophile, an extremophile, an acidophile, an alkaliphile, athermophile, psychrophile, halophile, or an osmophile.

In certain embodiments, the bacterium is an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In certain embodiments, bacterium is a mycobacteria, Clostridium tetani, Yersinia pestis, Bacillus anthracis, methicillin-resistant Staphylococcus aureus (MRSA), or Clostridium difficile.

### Fungal Antigens

In certain embodiments, the antigen comprises a fungal antigen or fragment or variant thereof. In certain embodiments, the fungus is Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Tumor Antigens

In certain embodiments, the antigen comprises a tumor antigen, including for example a tumor-associated antigen or a tumor-specific antigen. In the context of the present invention, “tumor antigen” or “hyperporoliferative disorder antigen” or “antigen associated with a hyperproliferative disorder” refer to antigens that are common to specific hyperproliferative disorders. In certain aspects, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

Tumor antigens are proteins that are produced by tumor cells that elicit an immune response, particularly T-cell mediated immune responses. In certain embodiments, the tumor antigen of the present invention comprises one or more antigenic cancer epitopes immunogenically recognized by tumor infiltrating lymphocytes (TIL) derived from a cancer tumor of a mammal. The selection of the antigen will depend on the particular type of cancer to be treated or prevented by way of the composition of the invention.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase. RU1, RU2 (AS), intestinal carboxyl esterase, mut hsp70-2, M-CSF, prostase, prostate-specific antigen (PSA), PAP. NY-ESO-1, LAGE-1a, p53, prostein, PSMA, Her2/neu, survivin and telomerase, prostate-carcinoma tumor antigen-1 (PCTA-1), MAGE, ELF2M. neutrophil elastase, ephrinB2, CD22, insulin growth factor (IGF)-I, IGF-II, IGF-I receptor and mesothelin.

In certain embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15: overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE. NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG. BCA225, BTAA, CA 125, CA 15-3\CA 27.29\BCAA, CA 195, CA 242, CA-50, CAM43, CD68\P1. CO-029, FGF-5, G250, Ga733\EpCAM, HTgp-175, M344, MA-50, MG7-Ag, MOV18, NB/70K, NY—CO-1, RCAS1, SDCCAG16, TA-90\Mac-2 binding protein\cyclophilin C-associated protein, TAAL6, TAG72, TLP, and TPS.

### Adjuvants

In certain embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2. LFA-3. M-CSF, G-CSF. IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88. IRAK, TRAF6, IkB, Inactive NIK, SAP K, SAP-I, JNK, interferon response genes, NFkB, Bax, TRAIL, TRAILrec, TRAILrecDRCS, TRAIL-R³, TRAIL-R⁴, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Methods

In one aspect, the present disclosure provides a method of treating, preventing, and/or ameliorating cancer in a subject, the method comprising administering to the subject the lipid nanoparticle (LNP) of the present disclosure and/or the pharmaceutical composition of the present disclosure.

In certain embodiments, the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer.

In certain embodiments, the cancer is at least one selected from the group consisting of leukemia (e.g., acute lymphoblastic leukemia, acute myeloid leukemia, chronic lymphocytic leukemia, chronic myeloid leukemia, hairy cell leukemia, and juvenile myelomonocytic leukemia), non-Hodgkin's lymphoma (e.g., diffuse large B-cell lymphoma, follicular lymphoma, mantle cell lymphoma, Burkitt lymphoma, and T-cell lymphoma), Hodgkin's lymphoma, multiple myeloma, myelodysplastic syndromes (MDS), and myeloproliferative neoplasms (MPNs).

In certain embodiments, the subject is further administered at least one additional agent or therapy useful for treating, preventing, and/or ameliorating cancer in a subject.

In certain embodiments, the at least one additional agent is selected from the group consisting of a small molecule anti-cancer agent and an antibody anti-cancer agent.

In certain embodiments, the subject is a mammal.

In certain embodiments, the mammal is a human.

In another aspect, the present disclosure provides a method of preparing a modified immune cell or precursor thereof, comprising contacting an immune cell or precursor thereof with the lipid nanoparticle (LNP) of the present disclosure.

In certain embodiments, the modified immune cell or precursor cell thereof is selected from the group consisting of an αβ T cell, a γδ T cell, a CD8+ T cell, a CD4+ helper T cell, a CD4+ regulatory T cell, an NK T cell, an NK cell, and any combination thereof.

In certain embodiments, the modified immune cell or precursor cell thereof is a T cell.

In certain embodiments, the modified immune cell or precursor cell thereof is a CD4+ T cell. In certain embodiments, the modified immune cell or precursor cell thereof is a CD8+ T cell.

The present invention provides methods of delivering an agent to an immune cell of a target subject. In some embodiments, the agent is a diagnostic agent to detect at least one marker associated with a disease or disorder. In some embodiments, the agent is a therapeutic agent for the treatment or prevention of a disease or disorder. Therefore, in some embodiments, the invention provides methods for diagnosing, treating or preventing a disease or disorder comprising administering an effective amount of a composition comprising one or more diagnostic or therapeutic agents, one or more adjuvants, or a combination thereof.

In some embodiments, the method provides for delivery of compositions for gene editing or genetic manipulation to a target immune cell of a subject to treat or prevent a disease or disorder. Exemplary diseases or disorders include, but are not limited to, pathogenic disease and disorders and cancer.

In some embodiments, the method provides immunity in the target subject to an infection, or a disease, or disorder associated with an infectious agent. The present invention thus provides a method of treating or preventing the infection, or a disease, or disorder associated with an infectious agent. For example, the method may be used to treat or prevent a viral infection, bacterial infection, fungal infection, or a parasitic infection, depending upon the type of antigen of the administered composition. Exemplary antigens and associated infections, diseases, and tumors are described elsewhere herein.

The present invention also relates in part to methods of treating cancer and diseases or disorders associated therewith in subjects in need thereof, the method comprising the administration of a composition comprising at least one immune cell targeted LNP comprising a nucleic acid molecule encoding a CAR specific for binding to an tumor antigen for the treatment of cancer, or a disease or disorder associated therewith. Exemplary cancers that can be treated using the compositions and methods of the invention include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In certain embodiments, the composition is administered to a target subject having an infection, disease, or cancer. In certain embodiments, the composition is administered to a subject at risk for developing an infection, disease, or cancer. For example, the composition may be administered to a subject who is at risk for being in contact with a virus, bacteria, fungus, parasite, or the like.

In certain embodiments, the method comprises administering an immune cell targeted LNP composition comprising one or more nucleic acid molecules for treatment or prevention of a disease or disorder. In certain embodiments, the one or more nucleic acid molecules encode a therapeutic agent for the treatment of the disease or disorder. In certain embodiments, the one or more nucleic acid molecules encode an agent for targeting T cells to an antigen expressed by a pathogen or a cancer cell (e.g., an mRNA molecule encoding a chimeric antigen receptor).

In certain embodiments, the compositions of the invention can be administered in combination with an additional therapeutic agent, an adjuvant, or a combination thereof. For example. In certain embodiments, the method comprises administering an LNP composition comprising a nucleic acid molecule encoding one or more agent for targeting an immune cell to a pathogen or a tumor cell of interest and a second LNP comprising a nucleic acid molecule encoding one or more adjuvants. In certain embodiments, the method comprises administering a single LNP composition comprising a nucleic acid molecule encoding one or more agent for targeting an immune cell to a pathogen or a tumor cell of interest and a nucleic acid molecule encoding one or more adjuvants.

In certain embodiments, the method comprises administering to subject a plurality of nucleoside-modified nucleic acid molecules encoding a plurality of agents for targeting an immune cell to a pathogen or a tumor cell of interest, adjuvants, or a combination thereof.

In certain embodiments, the method of the invention allows for sustained expression of the agent for targeting an immune cell to a pathogen or a tumor cell of interest or adjuvant, described herein, for at least several days following administration. However, the method, in certain embodiments, also provides for transient expression, as in certain embodiments, the nucleic acid is not integrated into the subject genome.

In certain embodiments, the method comprises administering nucleoside-modified RNA which provides stable expression of the agent for targeting an immune cell to a pathogen or a tumor cell of interest or adjuvant described herein.

Administration of the compositions of the invention in a method of treatment can be achieved in a number of different ways, using methods known in the art. In certain embodiments, the method of the invention comprises systemic administration of the subject, including for example enteral or parenteral administration. In certain embodiments, the method comprises intradermal delivery of the composition. In some embodiments, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding an agent for targeting an immune cell to a pathogen or a tumor cell of interest, adjuvant, or a combination thereof, described herein to practice the methods of the invention. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 10 nM and 10 μM in a mammal.

Typically, dosages which may be administered in a method of the invention to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 μg to about 10 mg per kilogram of body weight of the mammal. More preferably, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the present invention may be performed by single administration or boosted by multiple administrations.

In certain embodiments, the invention includes a method comprising administering one or more compositions encoding one or more agent for targeting an immune cell to a pathogen or a tumor cell of interest or adjuvants described herein. In certain embodiments, the method has an additive effect, wherein the overall effect of the administering the combination is approximately equal to the sum of the effects of administering each agent for targeting an immune cell to a pathogen or a tumor cell of interest or adjuvant. In other embodiments, the method has a synergistic effect, wherein the overall effect of administering the combination is greater than the sum of the effects of administering each agent for targeting an immune cell to a pathogen or a tumor cell of interest or adjuvant.

### Pharmaceutical Compositions

In another aspect, the present disclosure provides a pharmaceutical composition comprising the lipid nanoparticle (LNP) of the present disclosure and at least one pharmaceutically acceptable carrier. In certain embodiments, the composition further comprises at least one adjuvant. In certain embodiments, the composition is a vaccine.

Such a pharmaceutical composition may consist of at least one composition of the invention, in a form suitable for administration to a subject, or the pharmaceutical composition may comprise at least one composition, and one or more pharmaceutically acceptable carriers, one or more additional ingredients, or any combinations of these. At least one composition of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein. “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.010% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition: the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4.000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to. Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXPERIMENTAL EXAMPLES

Various embodiments of the present application can be better understood by reference to the following Examples which are offered by way of illustration. The scope of the present application is not limited to the Examples given herein.

### Materials and Methods

### Ionizable Lipid Synthesis

The C14-4 ionizable lipid was synthesized as follows. Seven equivalents of 1,2-epoxytetradecane (MilliporeSigma, Burlington, MA) were reacted with one equivalent of 2-{2-[4-(2-{[2-(2-aminoethoxy)ethyl]amino}ethyl)piperazin-1-yl]ethoxy}ethan-1-amine (Enamine, Kyiv, Ukraine) in ethanol for 2 days with vigorous stirring at 80° C. Afterwards the product was concentrated using a Rotovap R-300 (Buchi, New Castle, DE) and resuspended in ethanol before being used to formulate the lipid nanoparticles (LNPs).

mRNA Synthesis

Firefly luciferase, mCherry, and EGFP mRNAs were synthesized via in vitro transcription according to methods known to those of ordinary skill in the art. Briefly, linearized plasmids encoding the codon-optimized protein sequences were used as templates for T7 RNA polymerase (Megascript, Ambion). In the transcription reactions, N1-methyl-pseudouridine-5′-triphosphate (mlP, #N-1081, TriLink BioTechnologies, San Diego, CA) was substituted for uridine triphosphate and mRNAs were given 130 nucleotide-long 3′ poly(A) tails. Following transcription. RNAs were given 5′ Cap-1 with the m7G capping kit and 2′-O-methyltransferase (ScriptCap, CellScript). mRNAs were then purified via fast protein liquid chromatography (FPLC) with an Akta Purifier (GE healthcare). The correct synthesis of mRNAs was confirmed by denaturing or native agarose gel electrophoresis before storage at −80° C.

CAR mRNA was also synthesized via in vitro transcription. A linearized plasmid encoding a second-generation, human CD19-targeted CAR with a CD3ζ domain and a 4-1BB costimulatory domain followed by a 64 nucleotide-long 3′ poly(A) tail was used as the template for T7 RNA polymerase (New England Biolabs, Ipswich, MA, #E2040S). In the transcription reactions, N1-methyl-pseudouridine-5′-triphosphate (m1Ψ, #N-1081. TriLink BioTechnologies, San Diego, CA) was substituted for uridine triphosphate. Murine RNAse inhibitor (New England Biolabs, #M0314S) was added to prevent RNA degradation. Following transcription, plasmid template was digested with DNase I (New England Biolabs, #M0303S), and RNA was purified using the Monarch RNA Cleanup Kit (500 g) (New England Biolabs, #T2050L). Then, RNA was given 5′ Cap-1 using the vaccinia capping system (New England Biolabs #M2080S) and 2′-O-methyltransferase (New England Biolabs, #M0366) followed by a second purification using the Monarch RNA Cleanup Kit (500 μg) (New England Biolabs, #T2050L).

### Maleimide-Lipid Nanoparticle (Mal-LNP) Formulation

Mal-LNPs were formulated by mixing an aqueous mRNA solution with an ethanol lipid solution in a microfluidic device. The microfluidic device uses groove structures to induce chaotic mixing which results in the formation of homogenous LNPs. To form the aqueous phase, mRNA was resuspended in a 10 mM citrate buffer (pH=3) at 1 mg/mL. To form the ethanol phase, C14-4,1,2-dioleoyl-sn-glycero-3-phosphoethanolamine (DOPE, Avanti Polar Lipids, Alabaster, AL), cholesterol (MilliporeSigma), 1,2-dimyristoyl-sn-glycero-3-phosphoethanolamine-N-[methoxy(polyethylene glycol)-2000](ammonium salt) (C14-PEG2000, Avanti Polar Lipids), and 1,2-distearoyl-sn-glycero-3-phosphoethanolamine-N-[maleimide(polyethylene glycol)-2000](ammonium salt) (DSPE-PEG(2000) Maleimide, Avanti Polar Lipids) were combined at molar percentages of 41% C14-4, 30.8% DOPE, 25.6% cholesterol, 2.1% C14-PEG2000, and 0.4% DSPE-PEG(2000) Maleimide. The aqueous and ethanol phases were flowed into the microfluidic device at a 1:3 volume ratio (10:1 weight ratio of ionizable lipid:mRNA) using pump33DS syringe pumps (Harvard Apparatus, Holliston, MA). Upon exiting the device, mal-LNPs were dialyzed against PBS for two hours at 20 kDa molecular weight cutoff. Care was taken to keep all materials ribonuclease (RNase) free. Dialysis was performed inside a biosafety cabinet for LNPs that were used to treat T cells administered to mice.

### Antibody Cleavage and Disulfide Bond Reduction

Anti-human CD3 antibodies (BioXCell, InVivoMAb anti-human CD3, clone OKT-3, #BE0001-2) and anti-human CD28 antibodies (BioXCell, InVivoMAb anti-human CD28, clone 9.3, #BE0248) were cleaved with IdeZ protease (New England Biolabs, #P0770S) for two hours at 37° C. with gentle shaking at 300 rpm. Following cleavage, dithiothreitol (DTT) was added directly to the cleavage reaction at a volume ratio of 1 μL 20 mM DTT per 40 μL reaction mixture to reduce disulfide bonds. The resulting mixture was incubated for 30 minutes at room temperature with gentle shaking at 300 rpm. Following incubation, reduced and cleaved antibody mixtures were diluted in PBS and concentrated on pre-wet 10 kDa spin columns (abcam. #ab93349) to remove DTT. Antibody cleavage was confirmed by denaturing gel electrophoresis.

Activating LNP (aLNP) Formulation

The cleaved, reduced, and concentrated CD3 and CD28 antibody mixtures were directly added to dialyzed mal-LNPs at a stoichiometric equivalent of 1 antibody fragment per 1 maleimide binding site (-3.5 fragments were assumed per whole antibody). Suspensions were mixed via pipetting and then were incubated for 1 hour at room temperature with gentle shaking at 300 rpm. After incubation at room temperature. aLNPs were moved to 4° C., to finish reacting overnight with no shaking. The next day, aLNPs were purified by size exclusion chromatography with PBS as a running buffer (Sephadex®G-75 beads, Sigma-Aldrich, St. Louis. MO, #G7550) to remove the unbound Fc fragments and any unbound Fd′, LC, or F(ab′) fragments.

### LNP Characterization

LNPs were diluted 100× in 1×PBS. Dynamic light scattering (done by a Zetasizer Nano, Malvern Instruments, Malvern, UK) was used to measure polydispersity index (PDI) and hydrodynamic diameter (intensity-weighted z-average) in triplicate. Standard deviation (SD) of PDI was reported as the SD of the three measurements. SD of hydrodynamic diameter was calculated as SD=sqrt(average PDI×average z-average²)). An Infinite® 200 Pro M Plex plate reader with a NanoQuant plate (Tecan, Morrisville, NC) was used to measure mRNA concentration of LNPs by A260 absorbance.

### Primary Human T Cell Culture

PBMCs were obtained from de-identified consenting healthy human donors by leukapheresis and used a negative selection process to sort the cells into subcategories. For this work, CD4+ and CD8+ T cells were obtained from the HIC and mixed in a 1:1 ratio in RPMI-1640 medium supplemented with L-glutamine (Gibco), 10% (v/v) FBS (Gibco), and 1% (v/v) penicillin-streptomycin (Gibco) and maintained in a 37° C., 5% CO₂ humidified incubator. Control groups were activated with Dynabeads™ Human T-Activator CD3/CD28 (ThermoFisher, #11132D) using a 1:1 bead:cell ratio.

### Nalm6 Cell Culture

Nalm6 cells (ATCC #CRL-3273™) were cultured in RPMI-1640 medium supplemented with L-glutamine (Gibco), 10% (v/v) FBS (Gibco), and 1% (v/v) penicillin-streptomycin (Gibco) and maintained in a 37° C., 5% CO₂ humidified incubator. Cells were confirmed to be mycoplasma negative by use of a Cambrex MycoAlert Mycoplasma Detection Assay.

Luciferase mRNA Delivery to Primary Human T Cells Ex Vivo

Primary human T cells (bead-activated and non-activated) were plated in triplicate in clear-bottomed 96 well plates at 60,000 cells/60 μL/well. LNPs (mal-LNPs, αCD3-LNPs, αCD28-LNPs, αCD3-LNPs+αCD28-LNPs, and aLNPs) were used to administer 200 ng of luciferase mRNA to each well. After 24 hours, plates were centrifuged at 300 g for 7 minutes. Media was aspirated and cells were resuspended in 50 μL of 1× Reporter Lysis Buffer (Promega, Madison, WI, #E3971). 100 μL of Luciferase Assay Substrate (Promega, #E4550) was added in minimal light, and suspensions were mixed via pipette. After a 10-minute dark incubation, an Infinite® 200 Pro M Plex plate reader (Tecan) was used to measure luminescent signal. For each primary cell donor, luminescence was normalized to that donor's untreated cells.

mCherry and EGFP mRNA Delivery to Primary Human T Cells Ex Vivo

Primary human T cells (bead-activated and non-activated) were plated in triplicate or quadruplicate in clear-bottomed 96 well plates at 60,000 cells/60 L/well. LNPs (mal-LNPs and 1:50, 1:10, 1:3, 1:1, 3:1, 10:1, 50:1 aLNPs) were used to administer 200 ng of mCherry or EGFP mRNA to each well. After 24 hours, plates were centrifuged at 300 g for 7 minutes. Media was aspirated and cells were washed and resuspended in PBS. Expression of mCherry or EGFP was assessed using a BD LSRII flow cytometer. Data were analyzed using FlowJo 10.5.3 software. Standard gating was applied with doublet exclusion.

### Viability Assays

Primary human T cells (bead-activated and non-activated) were plated in triplicate in clear-bottomed 96 well plates at 60,000 cells/60 L/well. aLNPs were used to administer a fixed amount of mRNA to each well. After 24 hours, 60 L of CellTiter-Glo@Luminescent Cell Viability Assay Reagent (Promega. #G7572) was added per well in minimal light, and suspensions were mixed via pipette. After a 10-minute dark incubation, an Infinite® 200 Pro M Plex plate reader (Tecan) was used to measure luminescent signal. For each primary cell donor, luminescence (proportional to the amount of ATP, and therefore to the number of cells, in the culture) was normalized to that donor's untreated cells.

Co-Culture of aLNP Generated CAR T Cells with Nalm6 Cells

Primary human T cells (bead-activated and non-activated) were plated in 6 well plates at a density of 1×10⁶ cells/mL. LNPs (mal-LNPs and 1:10 aLNPs) were used to administer 600 ng of CAR mRNA per 60,000 cells. After 24 hours, a sample of cells was removed from each group and centrifuged at 300 g for 7 minutes. Media was aspirated, cells were resuspended in PBS, and stained with a rabbit anti-mouse FMC63 scFv monoclonal antibody conjugated to PE (Cytoart, Tucson, AZ, #200105) which binds to the specific CAR which was used. After staining, cells were washed and resuspended in PBS. CAR surface expression was assessed using a BD LSRII flow cytometer. Data were analyzed using FlowJo 10.5.3 software. Standard gating was applied with doublet exclusion.

After confirming cell-surface expression of CAR, both types of CAR T cells (bead+mal-LNP generated CAR T cells and 1:10 aLNP generated CAR T cells) were plated in co-culture with 25,000 Nalm6 (luciferase-expressing CD19+ human acute lymphoblastic leukemia) cells at various CAR T cell:Nalm6 cell ratios (1:1, 1:2, 1:4, 1:8, 1:16, 0:1) in triplicate in in clear-bottomed 96 well plates. After 48 hours, plates were centrifuged at 300 g for 7 minutes. Media was aspirated and cells were resuspended in 50 μL of 1× Reporter Lysis Buffer (Promega, #E3971). 100 μL of Luciferase Assay Substrate (Promega, #E4550) was added in minimal light, and suspensions were mixed via pipette. After a 10-minute dark incubation, an Infinite® 200 Pro M Plex plate reader (Tecan) was used to measure luminescent signal. The percent of Nalm6 cells killed per co-culture well was calculated as %=(luminescence of 0:1 well-luminescence of co-culture well)+luminescence of 0:1 well.

### Analysis of CD69, TNFα, and IFNγ Expression

To assess CD69 expression, primary human T cells (bead-activated and non-activated) were plated in 6 well plates at a density of 1×10⁶ cells/mL. LNPs (mal-LNPs and 1:10 aLNPs) were used to administer 400 ng of CAR mRNA per 60,000 cells. After 24 hours, cells were centrifuged at 300 g for 7 minutes. Media was aspirated, cells were resuspended in PBS, and stained with PE-CF594 mouse anti-human CD4 (BD Biosciences, Franklin Lakes, NJ, #562316), APC-Alexa Fluorf 750 mouse anti-human CD8 (Life Technologies. Carlsbad, CA, #MHCD0827), and Alexa Fluor®647 mouse anti-human CD69 (BioLegend, San Diego, CA, #310918). After staining, cells were washed and resuspended in PBS. Surface expression of CD4, CD8, and CD69 was assessed in triplicate using a BD LSRII flow cytometer. Data were analyzed using FlowJo 10.5.3 software. Standard gating was applied with doublet exclusion.

To quantify TNFα and IFNγ expression, primary human T cells (bead-activated and non-activated) were plated in 6 well plates at a density of 1×10⁶ cells/mL. LNPs (mal-LNPs and 1:10 aLNPs) were used to administer 400 ng of CAR mRNA per 60,000 cells. After 24 hours, supernatants were collected and LEGEND MAX™ human TNFα and IFNγ ELISA kits (BioLegend, #430207 and #430107) were used to quantify TNFα and IFNγ expression following manufacturer protocol with an Infinite® 200 Pro M Plex plate reader (Tecan).

### In Vivo Leukemia Xenograft Model

In order to obtain enough T cells for this experiment, primary female human T cells procured from the HIC at Penn were expanded—on day −5, a 1:1 mixture of CD4+:CD8+ T cells was plated at a density of 1×10⁶ cells/mL with recombinant human IL-2 at 50 U/μL (Corning, Corning. NY, #BD354043) and Dynabeads™ Human T-Activator CD3/CD28 at a 1:1 bead:cell ratio. On day −1 (after 4 days of expansion) beads were removed with a MojoSort™ magnet and 1:10 aLNPs were used to administer 400 ng of CAR mRNA per 60,000 cells to a portion of the cells. After 24 hours (day 0), samples of aLNP-treated and aLNP-untreated cells were removed and centrifuged at 300 g for 7 minutes. Media was aspirated, cells were resuspended in PBS, and stained with a rabbit anti-mouse FMC63 scFv monoclonal antibody conjugated to PE (Cytoart, #200105). After staining, cells were washed and resuspended in PBS. CAR surface expression was assessed using a BD LSRII flow cytometer in triplicate. Data were analyzed using FlowJo 10.5.3 software. Standard gating was applied with doublet exclusion.

Concurrently, on day −4, 250,000 luciferase-expressing CD19+Nalm6 cells were injected in 100 μL sterile PBS into the tail veins of fifteen female NOD.Cg-Prkdc^{scid}Il2rg^{tmlWjl}/SzJ (NSG) mice. On day 0, after confirming establishment of similar tumor burden in all mice, 2×10⁶ CAR T cells generated with 1:10 aLNPs were administered in 100 μL sterile PBS via tail vein injection to 5 mice. As control groups. 5 mice received tail vein injections of untransfected T cells, and 5 mice received tail vein injections of PBS. On days 2 and 5, 1:10 aLNPs were used to transfect fresh CAR T cells (from the original culture of primary human T cells). On days 3 and 6, after confirming CAR expression by flow cytometry, CAR T cells, untransfected T cells, and PBS were re-injected.

Periodically throughout the treatment (days 0, 2, 3, 5, 6, 7, 9, 11, and 14) mice were intraperitoneally injected with 200 μL of D-luciferin potassium salt (Biotium, Fremont, CA) in PBS at 15 mg/mL. After 10 minutes, mice were anesthetized with 2.5% isoflurane and a Lumina S3 in vivo imaging system (IVIS, PerkinElmer, Waltham, MA) was used to capture bioluminescence images. Living Image 4.7.3 Software (PerkinElmer) was used to quantify total flux for each mouse at each imaging time point. After day 14, mice were monitored for survival and euthanized with CO₂ at the first sign of illness. The mice were housed, and all animal work was done at the Stem Cell and Xenograft Core (RRID:SCR_010035) at the University of Pennsylvania to maintain a sterile environment, under a protocol approved by the University of Pennsylvania's Institutional Animal Care and Use Committee (IACUC protocol #806540). The animal housing facility was maintained at 22±2° C. 12-hour dark/light cycle, and 40-70% air humidity.

### Statistical Analysis

Data are presented as mean±standard deviation. Differences between means were assessed by ordinary or repeated measures one-way or two-way analyses of variance (ANOVAs) with post hoc t tests using either Tukey's or Sidak's correction for multiple comparisons. Differences between survival profiles were assessed using pairwise Log-rank tests with Bonferroni corrections for multiple comparisons. Statistical analyses were completed using GraphPad Prism 9.5.1 with significance level α=0.05.

### Example 1: Formulation and Characterization of Exemplary Activated Lipid Nanoparticles (aLNPs)

LNPs, including LNPs for T cell applications, typically comprise four components: (1) an ionizable lipid, which is neutrally charged at physiological pH but positively charged in acidic pH, to aid in endosomal escape; (2) a helper phospholipid to promote LNP structure and organization; (3) cholesterol, or a derivative thereof, to provide LNP stability; and (4) a polymer conjugated lipid (e.g., lipid-anchored polyethylene glycol), to encourage LNP self-assembly and reduce LNP aggregation. It has been previously shown that the inclusion of maleimide functional groups on the LNP surface enables covalent conjugation of antibody fragments which have had disulfide bonds reduced to afford reactive thiol groups.

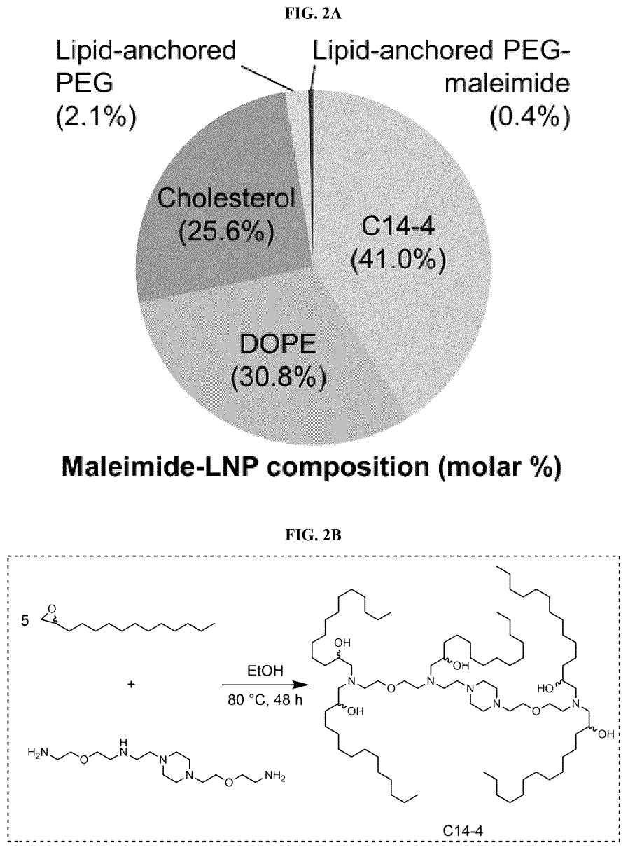

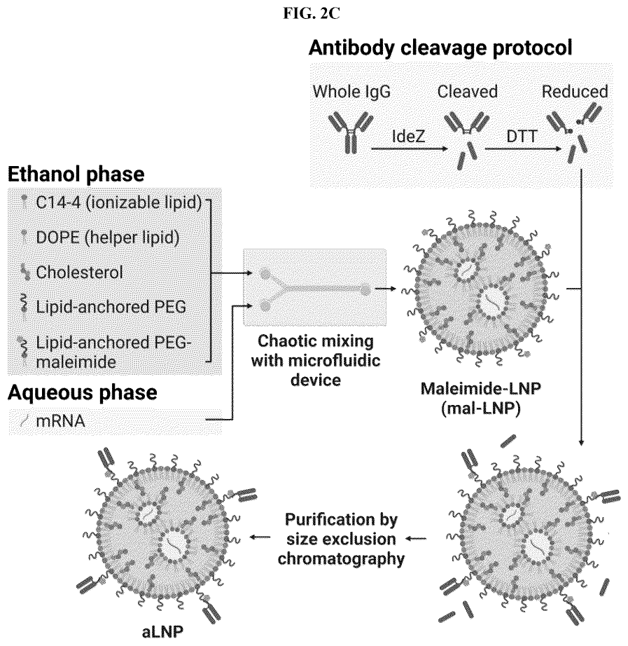

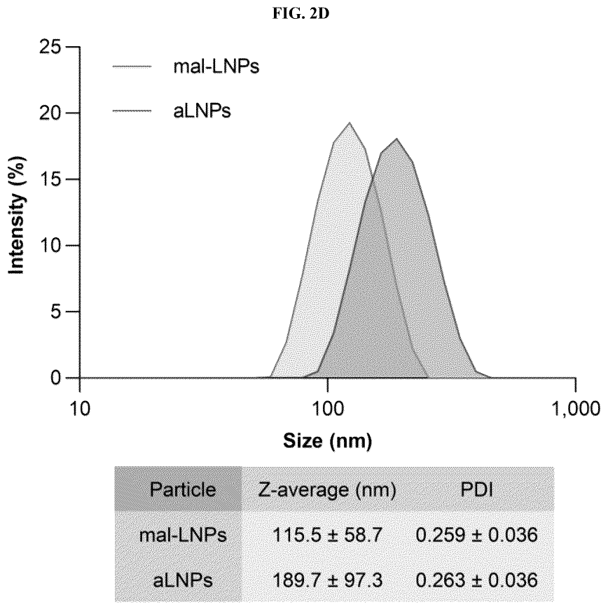

In one aspect, the present disclosure describes the use of maleimide-functionalized LNPs (e.g., wherein LNPs were formulated with a fraction of the lipid-anchored PEG replaced by lipid-anchored PEG-maleimide) (FIG. 2A). An S_{N}2 reaction was employed to synthesize the ionizable lipid C14-4 by reacting an exemplary polyamine core (4) with an excess of 1,2-epoxytetradecane (C14) (FIG. 2B). Then, an exemplary LNP was prepared by combination of ionizable lipid C14-4 with the helper phospholipid dioleoylphosphatidylethanolamine (DOPE), cholesterol, lipid-anchored PEG (C14-PEG), lipid-anchored PEG-maleimide (DSPE-PEG-maleimide), and mRNA in a microfluidic mixing device to form mal-LNPs.

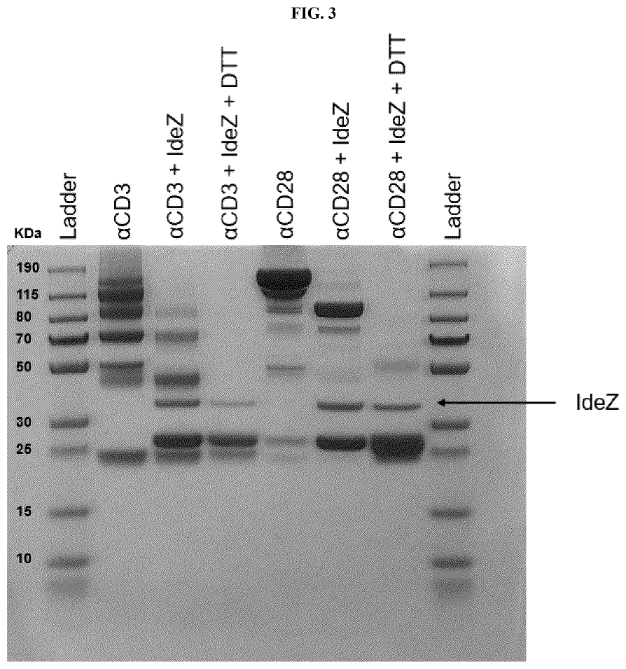

Separately, human CD3 and CD28 antibodies were enzymatically cleaved with IdeZ into F(ab′)₂ and Fc fragments, and then treated with dithiothreitol (DTT) to reduce the disulfide bonds on the F(ab′)₂ fragments to thiol groups, producing a mixture of Fd′, LC, and F(ab′) fragments (FIG. 3). The cleaved and reduced CD3 and CD28 antibody fragments were added to the mal-LNPs for surface conjugation via a thiol-maleimide reaction to produce aLNPs. Following conjugation, aLNPs were purified by size exclusion chromatography to remove the unbound Fc fragments as well as any unbound Fd′, LC, or F(ab′) fragments (FIG. 2C).

Dynamic light scattering (DLS) was used to characterize mal-LNPs before and after antibody fragment conjugation (FIG. 2D). The hydrodynamic diameter, measured as intensity weighted z-average, showed an increase in particle size from 115.5 nm for mal-LNPs to 189.7 nm for aLNPs. This increase in size between the two particles has been interpreted herein as confirmation that antibody fragments were successfully covalently conjugated to the aLNP surface. It was also observed that exemplary LNPs maintained their polydispersity after antibody conjugation (e.g., PDI of 0.259 for mal-LNPs and 0.263 for aLNPs), which suggests that size exclusion chromatography worked well. Accordingly, the maleimide-thiol conjugation strategy represents an applicable strategy for the modification of LNPs can be successfully applied to prepare aLNPs.

In certain embodiments, encapsulation efficiency was assessed for certain exemplary LNPs via RiboGreen assay. An encapsulation efficiency of 94.3% was observed for LNPs, formulated as described herein, comprising maleimide functionalized lipids (mal-LNP). An encapsulation efficiency of 83.0% was observed for 1:10 aLNPs, formulated as described herein.

Antibody fragment concentration in exemplary aLNPs was assessed via conjugation of DyLight 550 to aCD3 fragments and DyLight 755 to aCD28 fragments, according to manufacturer protocols. Antibody fragment concentration in exemplary 1:10 aLNPs were found to be 12.7 ng/μL and 109 ng/μL for aCD3 and aCD28 antibody fragments, respectively.

### Example 2: Exemplary aLNPs Efficiently Transfect Primary Human T Cells without Activating Beads Ex Vivo

An ex vivo screen was next devised which would allow: (1) evaluation of the ability of aLNPs to deliver their mRNA cargo to primary human T cells in the presence and absence of activating beads, and (2) exploration of the individual effects of conjugated CD3 and CD28 antibody fragments on LNP-mediated mRNA delivery. For this screen, LNPs were formulated to encapsulate mRNA encoding for the established model cargo luciferase, an enzyme which produces luminescence proportional to its concentration upon addition of the luciferin substrate.

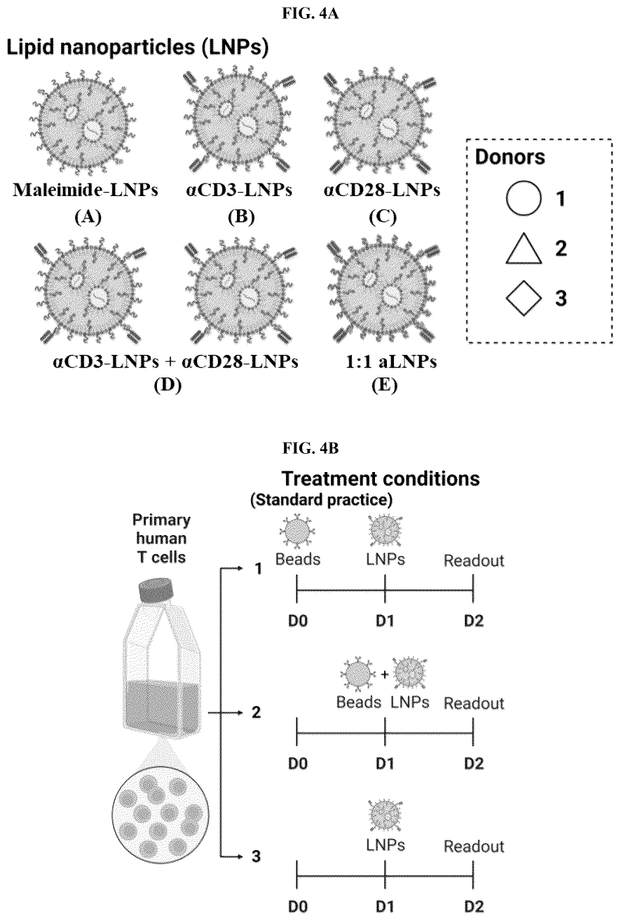

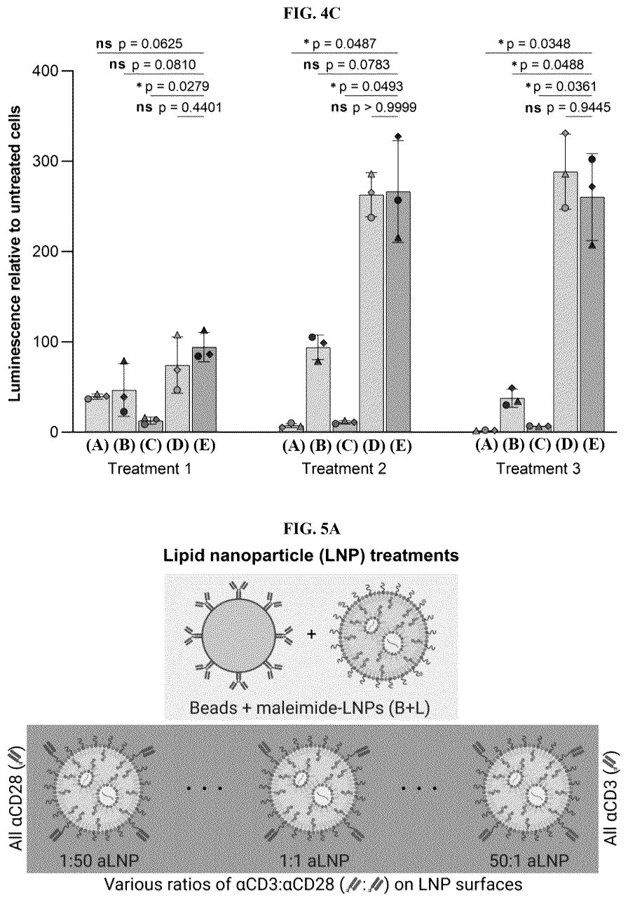

For the initial screen, five LNP groups were explored: (i) mal-LNPs, (ii) mal-LNPs conjugated to CD3 antibody fragments (αCD3-LNPs), (iii) mal-LNPs conjugated to CD28 antibody fragments (αCD28-LNPs), (iv) an equal-part mixture of aCD3-LNPs and aCD28-LNPs, and (v) mal-LNPs conjugated with a 1:1 ratio of CD3:CD28 antibody fragments (aLNPs). Each group was added to primary human T cells according to one of three different treatment schemes that were performed in parallel on cells from the same donor (FIGS. 4A-4C).

For treatment 1, activating beads were added to the cells on the day the cells were received from the donor (day 0) and LNPs were added 24 hours later (day 1). For treatment 2, activating beads and LNPs were both added on day 1. For treatment 3, LNPs were added on day 1 and activating beads were not added. In all three treatments, luciferin was added to the cells and luminescence was assessed on day 2, 24 hours after dosing with LNPs. The full screen was completed on primary human T cells from three different donors.

The results of the screen highlight that aLNPs alone are a promising alternative to the traditional activating bead and transfection agent workflow (FIGS. 4A-4C). Compared to the standard workflow of mal-LNPs in treatment 1, it was observed that the aLNPs and aCD3-LNPs+αCD28-LNPs administered in treatment 3 resulted in 6.6-fold and 7.3-fold increases in luminescence, respectively, while the remaining LNP groups resulted in decreased luminescence. This indicates that aLNPs or αCD3-LNPs+aCD28-LNPs can potently transfect primary human T cells with mRNA in the absence of activating beads, while LNPs without antibody fragments or with only CD3 or only CD28 antibody fragments cannot, thereby highlighting the importance of providing T cells with both primary and costimulatory activation signals.

Further, it was noted that there were no statistically significant differences in luminescence between the aLNP group and the αCD3-LNPs+αCD28-LNP group in any of the treatment conditions, suggesting that it does not matter whether CD3 and CD28 antibody fragments are on the same or separate LNPs as both strategies are similarly effective at facilitating LNP uptake and mRNA translation. Unexpectedly, it was also noted that the aLNP groups for treatment 2 and treatment 3 both had 2.8-fold higher luminescence than the aLNP group for treatment 1. Without wishing to be bound by any theory, it has been reasoned that pre-treatment with activating beads sterically inhibits aLNP uptake resulting in lower luminescence. Finally, it was observed that aLNPs perform just as well in treatment 3 as they do in treatment 2, indicating that activating beads are neither required for nor do they enhance mRNA delivery by aLNPs, thus validating aLNPs as a stand-alone T cell transfection reagent. Thus, aLNPs provide a one-step method to transfect primary human T cells without the need for activating beads.

### Example 3: Exemplary aLNP CD3 to CD28 Antibody Fragment Ratio Optimization Enhances mRNA Delivery Ex Vivo

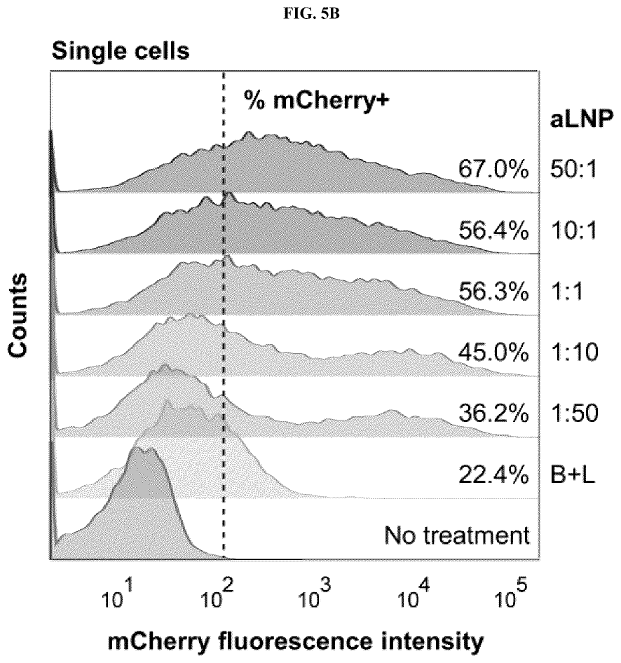

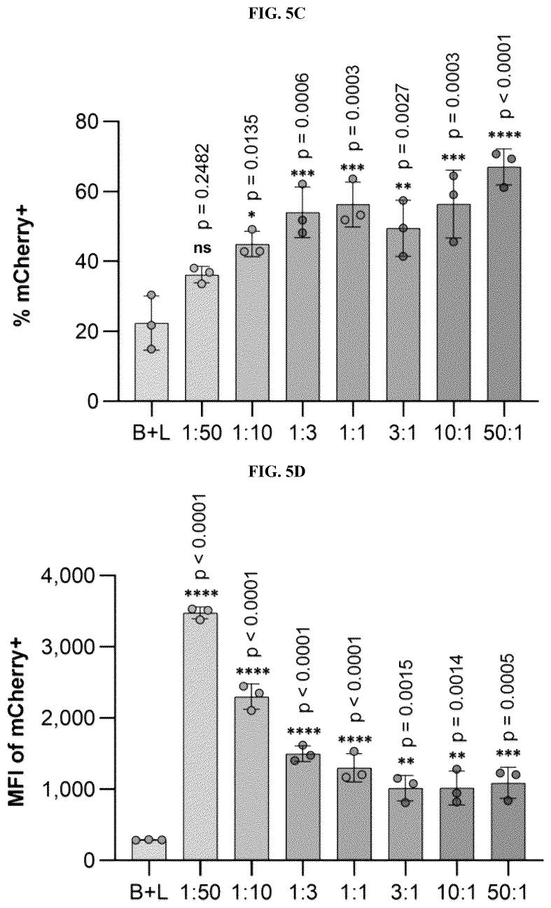

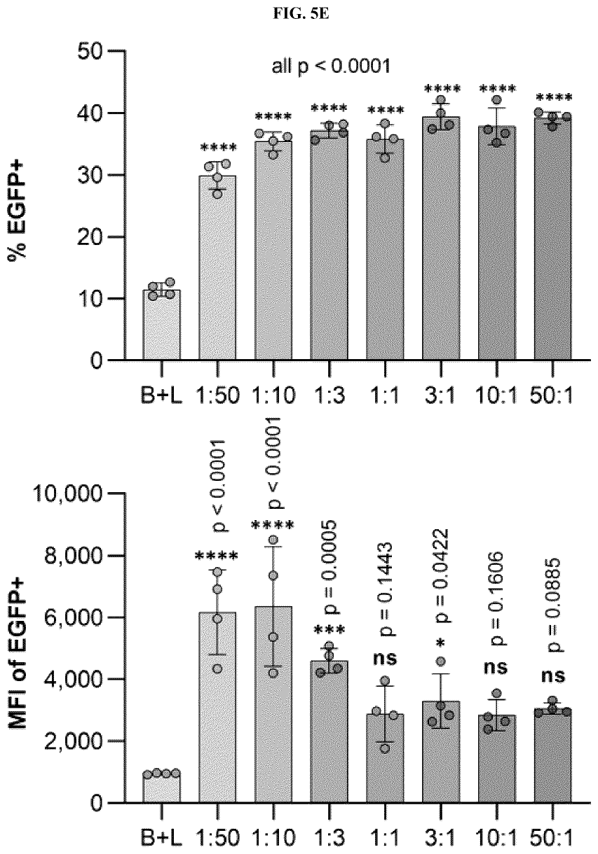

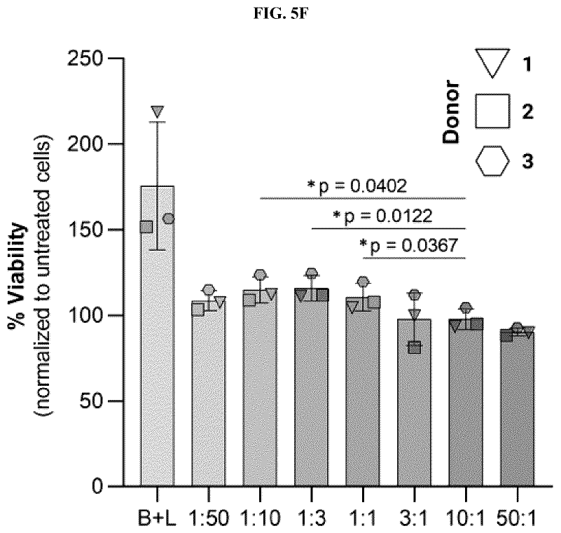

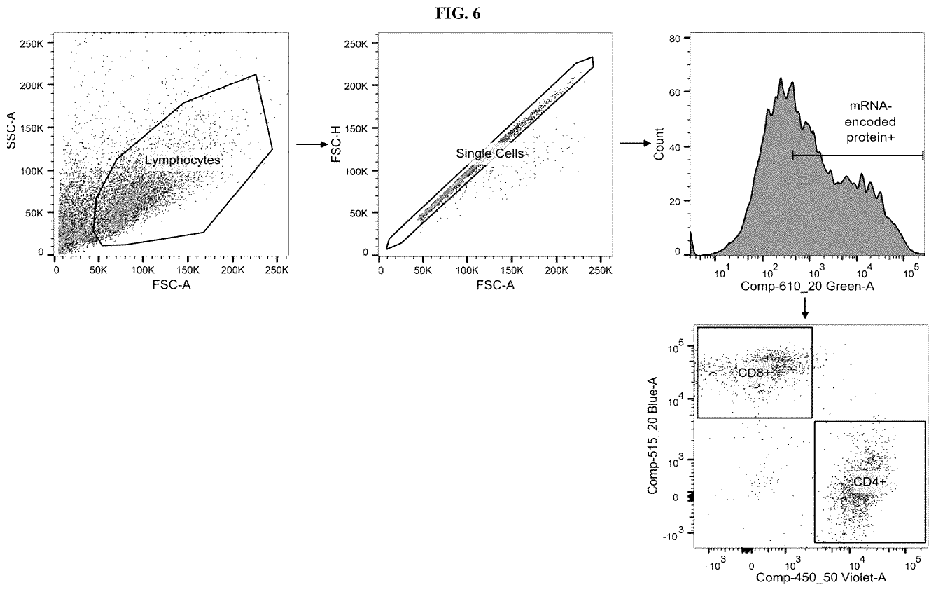

After confirming that aLNPs were able to transfect primary human T cells with mRNA in the absence of activating beads, it was next evaluated whether adjusting the ratio of CD3:CD28 antibody fragments on the aLNP surface would impact transfection. Primary human T cells were treated with beads+mCherry mRNA mal-LNPs or mCherry mRNA aLNPs with 1:50, 1:10, 1:3, 1:1, 3:1, 10:1, and 50:1 ratios of CD3:CD28 antibody fragments on their surfaces (FIG. 5A; FIG. 6). It was observed that all aLNP variants resulted in higher mCherry transfection efficiencies than beads+mal-LNPs, highlighting the benefit of conjugating CD3 and CD28 antibody fragments to the LNP surface, regardless of ratio. Further, an increase in transfection efficiency was noted with a higher ratio of CD3 antibody fragments, as 1:50 aLNPs resulted in a 36.2% transfection efficiency whereas 50:1 aLNPs resulted in a 67.0% transfection efficiency. Additionally, a steep decrease in the mean fluorescence intensity (MFI) of the mCherry+ cells was observed as the ratio of CD3 antibody fragments increased, with 1:50 aLNPs facilitating mCherry+ cells with an MFI of 3474 and 50:1 aLNPs resulting in mCherry+ cells with an MFI of 1089 (FIGS. 5B-5D).

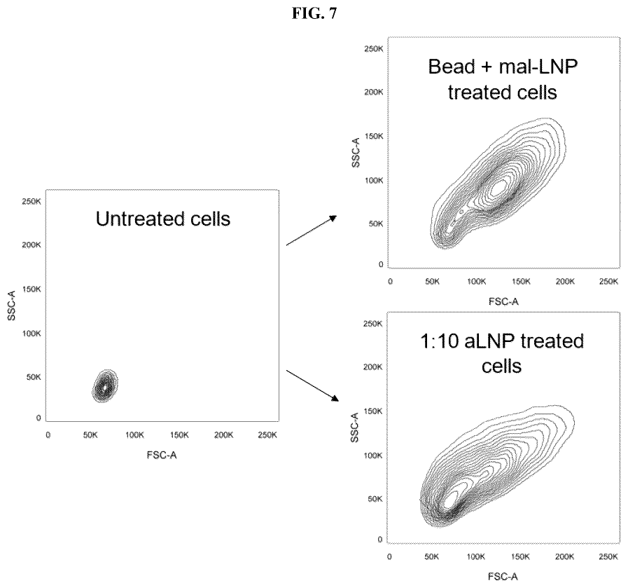

To confirm these results, the screen was repeated with aLNPs encapsulating mRNA encoding EGFP. As with mCherry, it was observed that all aLNP variants resulted in higher EGFP transfection efficiencies than beads+mal-LNPs. Again, an increase in transfection efficiency was observed with a higher ratio of CD3 antibody fragments, as 1:50 aLNPs led to a 29.9% transfection efficiency and 50:1 aLNPs resulted in a 39.2% transfection efficiency. Also, a decrease in MFI of the EGFP+ cells was again observed as the ratio of CD3 antibody fragments increased, from an MFI of 6165 for 1:50 aLNPs to an MFI of 3056 for 50:1 aLNPs (FIG. 5E and FIG. 7; Table 1). Therefore, it was reasoned that aLNPs with fewer CD3 than CD28 antibody fragments result in lower transfection efficiencies with more potent expression of the mRNA-encoded protein in the transfected population. Conversely, aLNPs with more CD3 than CD28 antibody fragments result in higher transfection efficiencies with less potent protein expression in the transfected population.

TABLE 1
Z-average and polydispersity index measurements
for certain exemplary LNPs

| LNP | Z-average (nm) | Polydispersity Index (PDI) |
|---|---|---|
| mal-LNPs | 98.8 ± 57.5 | 0.338 ± 0.047 |
| 1:50 aLNPs | 269.0 ± 140.3 | 0.272 ± 0.112 |
| 1:10 aLNPs | 254.4 ± 126.0 | 0.245 ± 0.047 |
| 1:3 aLNPs | 219.5 ± 62.2 | 0.080 ± 0.051 |
| 1:1 aLNPs | 224.9 ± 143.6 | 0.408 ± 0.095 |
| 3:1 aLNPs | 225.8 ± 103.5 | 0.210 ± 0.094 |
| 10:1 aLNPs | 189.5 ± 59.8 | 0.098 ± 0.025 |
| 50:1 aLNPs | 188.8 ± 55.9 | 0.088 ± 0.033 |
| αCD3 LNP | 176.4 ± 60.5 | 0.118 ± 0.032 |
| αCD28 LNP | 278.5 ± 171.3 | 0.378 ± 0.009 |

In the clinic, activating beads are used not only to activate T cells, but also to expand their population. Therefore, it was investigated whether aLNPs could drive cell expansion, and whether some CD3:CD28 antibody fragment ratios would drive more expansion than others. To test this. 1:50, 1:10, 1:3, 1:1, 3:1, 10:1, and 50:1 aLNPs were added to primary human T cells. Although activating beads did drive more cell expansion than aLNPs, a subtle curve in aLNP-induced cell expansion was noted across the aLNP variants, with 1:10 and 1:3 aLNPs facilitating the highest mean-fold change over untreated cells, 1.15-fold and 1.16-fold increases, respectively (FIG. 5F). Therefore, it appears that increasing the proportion of CD3 antibody fragments on the aLNP surface generally increased the transfection efficiency, but at the cost of driving less expansion.

These ex vivo results informed the selection of an optimal aLNP for continued investigation. In one aspect, an ideal aLNP platform would demonstrate a high MFI as previous studies have found that low CAR expression limits the efficacy of CAR T cells. The aLNPs would also demonstrate a high transfection efficiency as this would maximize the CAR T cells produced from the limited resource of isolated patient T cells. Further, the aLNPs would induce T cell expansion rather than toxicity. With these parameters in mind, 1:10 aLNPs were selected for further evaluation and/or optimization herein, as it was hypothesized that these LNPs would provide an optimal balance of the three factors described elsewhere herein.

### Example 4: Anti-CD19 CAR T Cells Generated with aLNPs Perform Potent Leukemia Cell Killing Ex Vivo

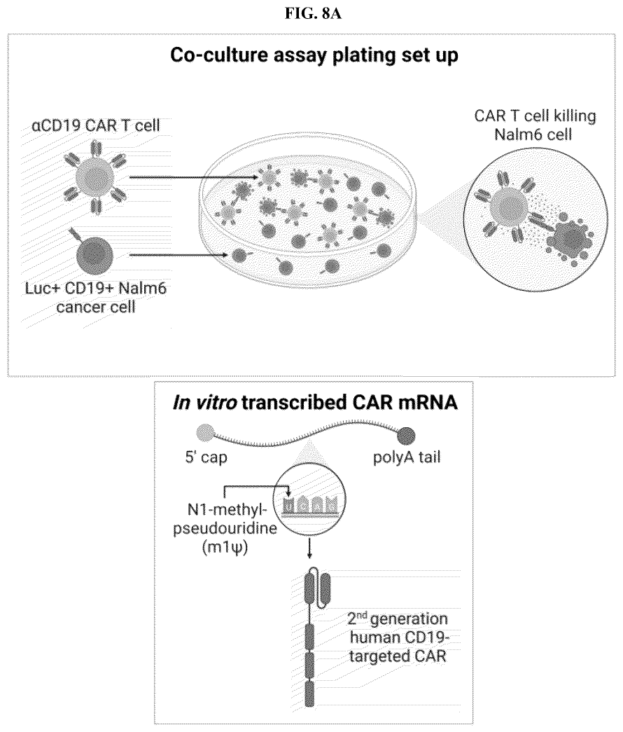

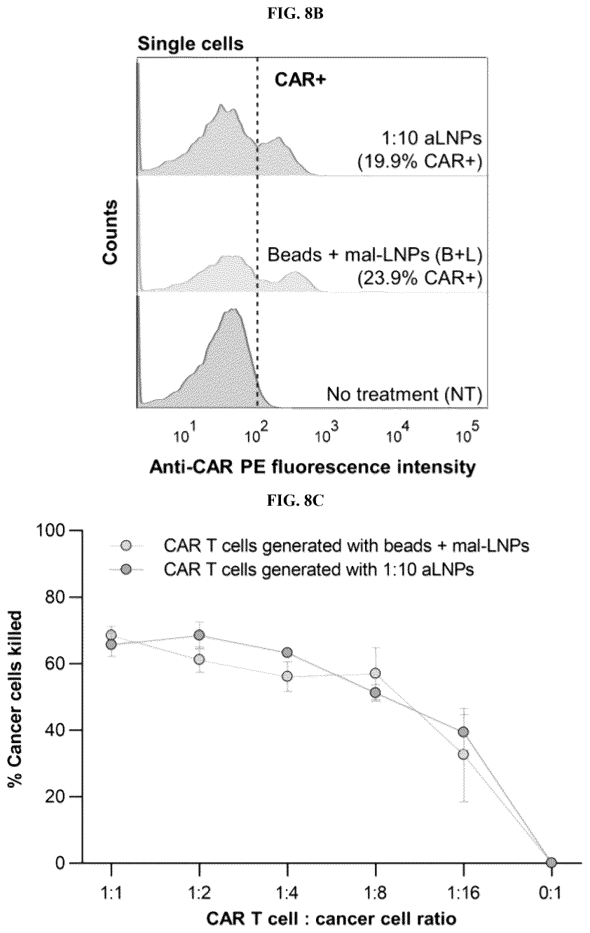

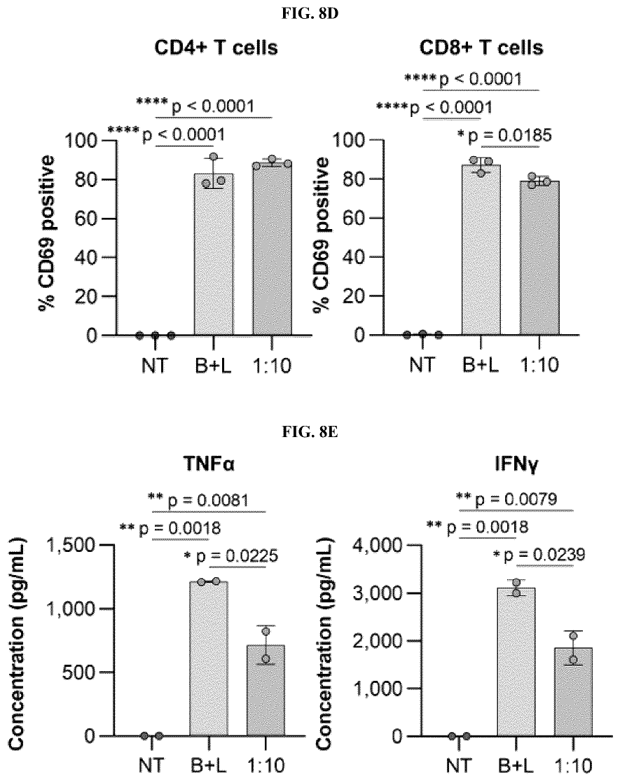

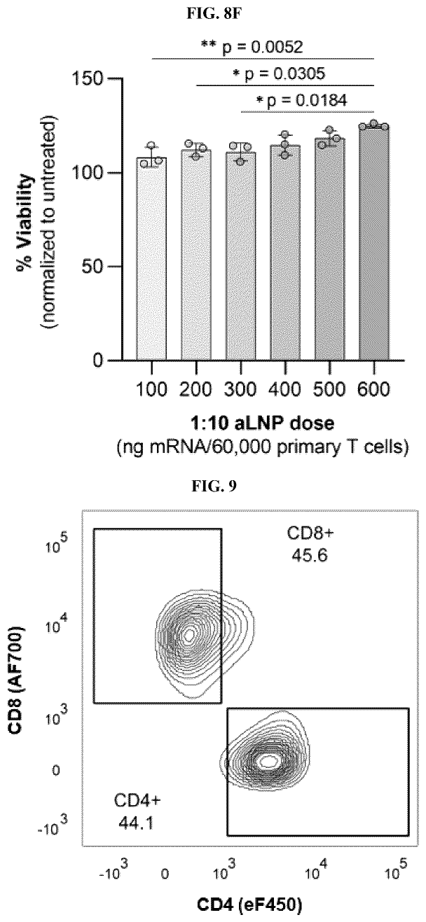

Next, a tumor cell co-culture experiment was performed to assess whether 1:10 aLNPs could be used to generate functional CAR T cells. 1:10 aLNPs were formulated to encapsulate mRNA encoding a second-generation human CD19-targeted CAR and administered to primary human T cells along with a control of beads+mal-LNPs. The cells treated with 1:10 aLNPs were 19.9% CAR positive while the cells treated with beads+mal-LNPs were 23.9% CAR positive (FIGS. 8A-8C). This is a lower positivity rate for the 1:10 aLNPs than was observed for mCherry and EGFP; however, this could be due to donor-to-donor variability, or to the increased complexity of CAR expression as compared to the reporter fluorescent proteins. CAR T cells from each production method were mixed with luciferase-expressing Nalm6 CD19+ human acute lymphoblastic leukemia cells in various CAR T cell:Nalm6 cell ratios. The percentage of Nalm6 cells killed was quantified after 48 hours and found to range from approximately 70% to 35% across the CAR T cell:Nalm6 cell ratios. Within each of the ratios, no statistically significant difference was found between CAR T cells generated with beads+mal-LNPs and CAR T cells generated with 1:10 aLNPs (FIGS. 8A-8C). Therefore, it was concluded that CAR T cells produced by the two methods are equivalently effective, validating bead-free CAR T cell production using aLNPs.

Next, whether CAR T cells produced by these two methods differ in their activation states or in their secretion of effector cytokines prior to exposure to target cells was evaluated. To assess activation status, CD69 expression on the surfaces of primary human T cells that were untreated, treated with beads+mal-LNPs, or treated with 1:10 aLNPs was quantified. CD69 is an early marker of T cell activation. CD4+ and CD8+ T cells treated with beads+mal-LNPs and 1:10 aLNPs had significantly higher percentages of surface expression of CD69 compared to untreated cells, with increases from approximately 0.1% for untreated cells to approximately 85% for bead+mal-LNP and 1:10 aLNP treated cells, indicating that both beads and aLNPs strongly activate T cells (FIG. 8D).

To assess secretion of effector cytokines, the concentration of tumor necrosis factor alpha (TNFα) and interferon gamma (IFNγ), two pro-inflammatory cytokines secreted by activated T cells, in the media of primary human T cells that were untreated, treated with beads+mal-LNPs, or treated with 1:10 aLNPs was quantified. For both TNFα and IFNγ, cells treated with beads+mal-LNPs and 1:10 aLNPs had significantly higher cytokine expression than untreated cells (TNFα: 1211 μg/mL and 714 μg/mL, respectively, and IFNγ: 3107 μg/mL and 1853 μg/mL, respectively), again implying that both beads and aLNPs activated the cells (FIG. 8E). The cells treated with beads+mal-LNPs expressed significantly more TNFα and IFNγ than the cells treated with 1:10 aLNPs but, since the two types of CAR T cells performed equivalent cancer cell killing, this difference may not be critical to therapeutic efficacy. Furthermore, it was reported in a clinical trial that the severity of cytokine release syndrome correlated with TNFα and IFNγ serum levels. Therefore, it is possible that the lesser cytokine release with equivalent cancer cell killing observed for aLNPs is advantageous over beads+mal-LNPs as it could result in less-severe cytokine release syndrome upon infusion of CAR T cells.

Lastly, to determine the most appropriate 1:10 aLNP dose to use, primary human T cells were treated with escalating doses of 1:10 aLNPs, ranging from 100 to 600 ng of mRNA per 60,000 cells and assessed cell viability/expansion 24 hours later. No toxicity, even at the highest dose, was observed. Instead, it was found that increasing the dose increased T cell expansion, with the 100 ng dose leading to a 1.08-fold increase in cell number compared to untreated cells and the 600 ng dose leading to a 1.25-fold increase. Statistical analysis revealed that the 600 ng dose resulted in significantly higher viability/expansion than the 100 ng, 200 ng, or 300 ng doses but not significantly higher viability/expansion than the 400 ng or 500 ng doses (FIG. 8F). Therefore, the 400 ng dose was selected for further consideration, to maximize T cell expansion while minimizing dosage.

### Example 5: Adoptive Transfer of Anti-CD19 CAR T Cells Generated with aLNPs Reduces Tumor Burden In Vivo

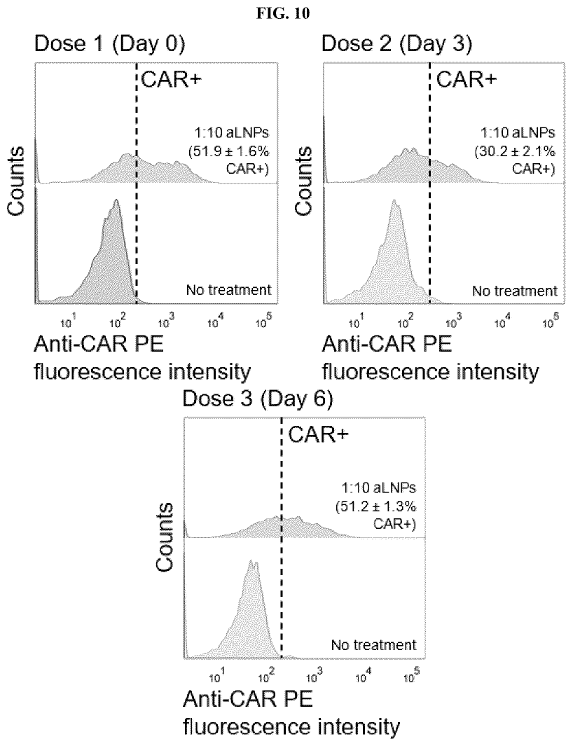

The efficacy of 1:10 aLNP generated CAR T cells were tested in a murine xenograft model of leukemia. Due to its transience, mRNA CAR T cell therapy is indicated in cases where tumor burden is low. Therefore, a mouse model was employed that mimics low leukemic burden. NOD.Cg-Prkd^{scid}Il2rg^{tm1Wjl}/SzJ (NSG) immunodeficient mice were inoculated with luciferase-expressing CD19+ Nalm6 cells. Four days later, 2×10⁶ anti-CD19 CAR T cells generated with aLNPs were administered to each mouse. To counteract the transience of mRNA expression, 2×10⁶ aLNP generated CAR T cells were re-administered 3 and 6 days following the initial administration (FIG. 11A and FIGS. 9-10).

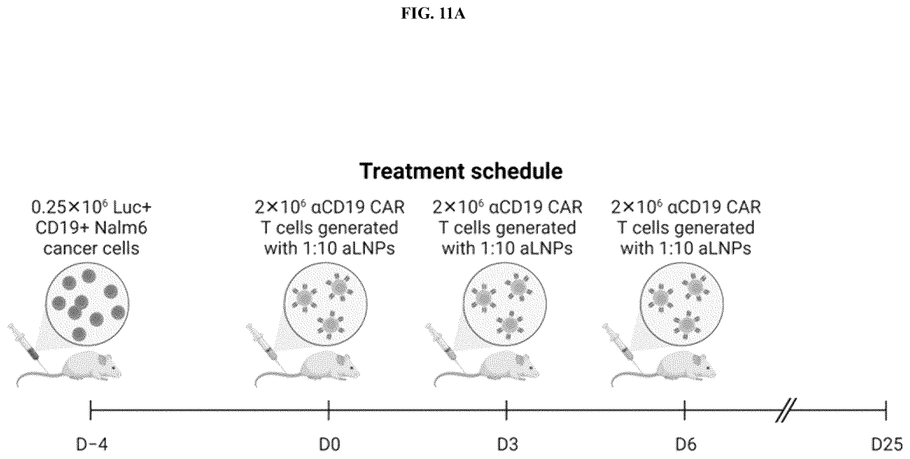

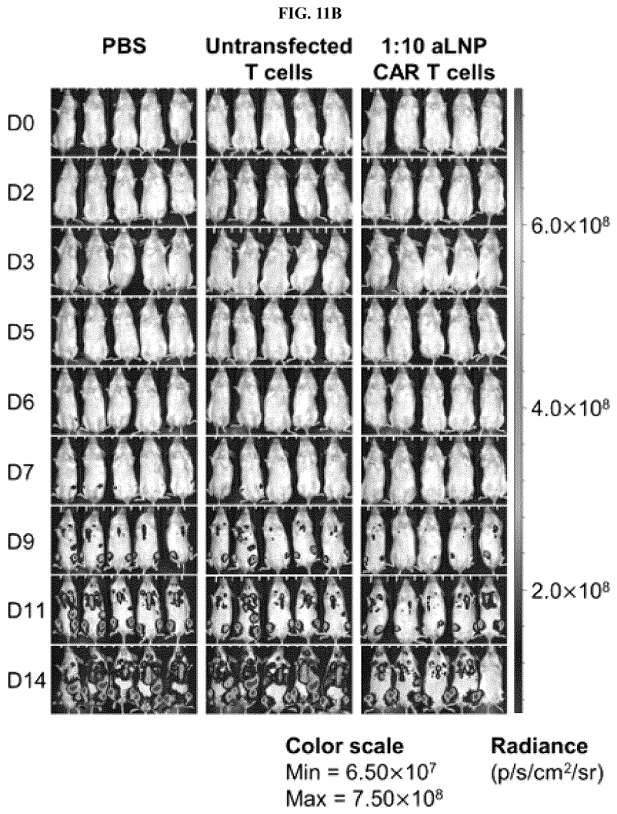

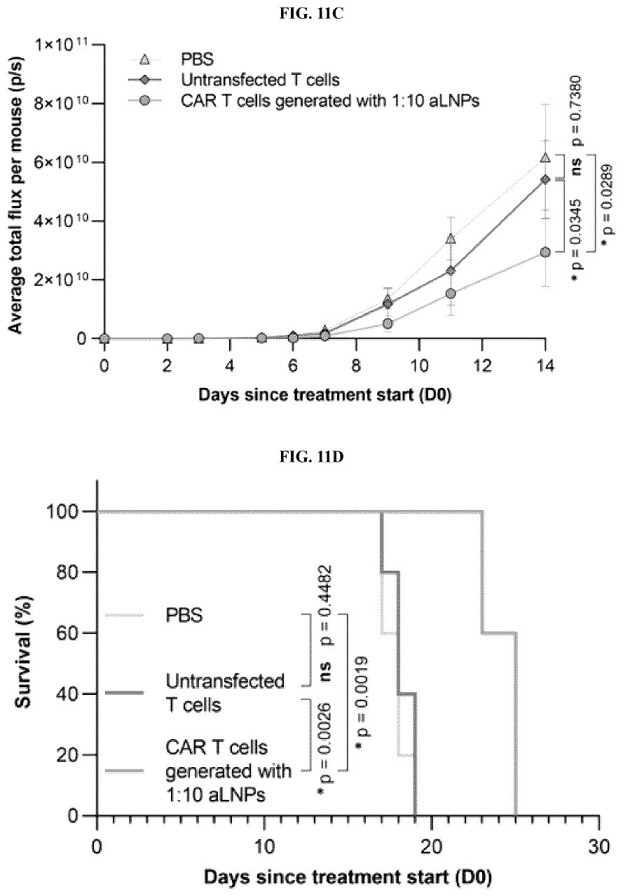

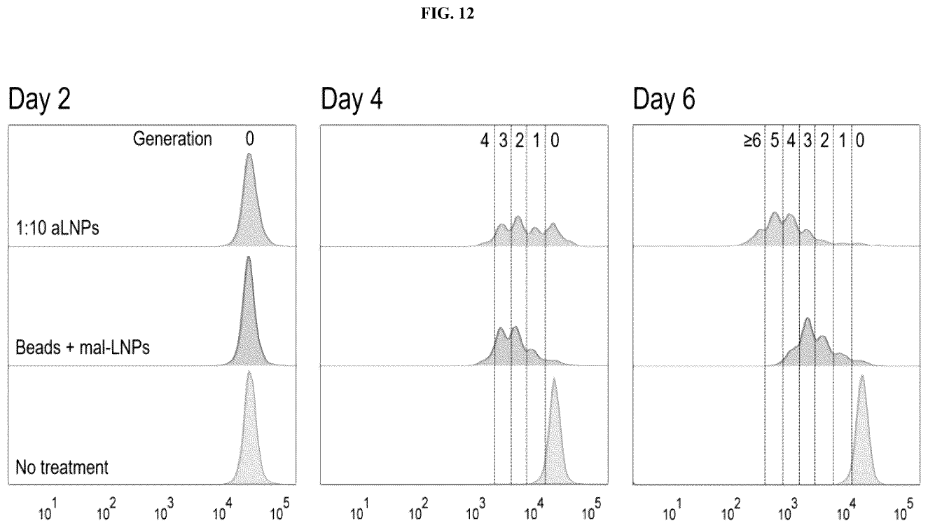

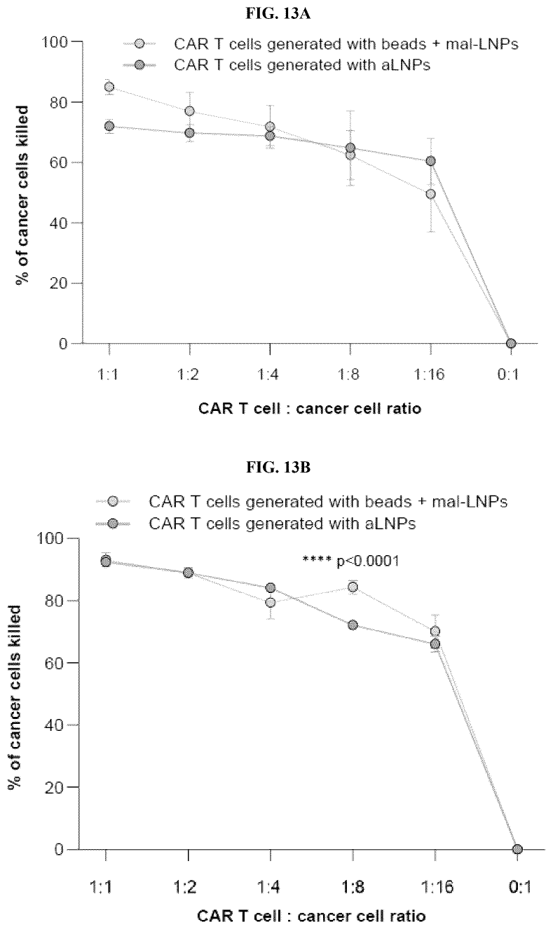

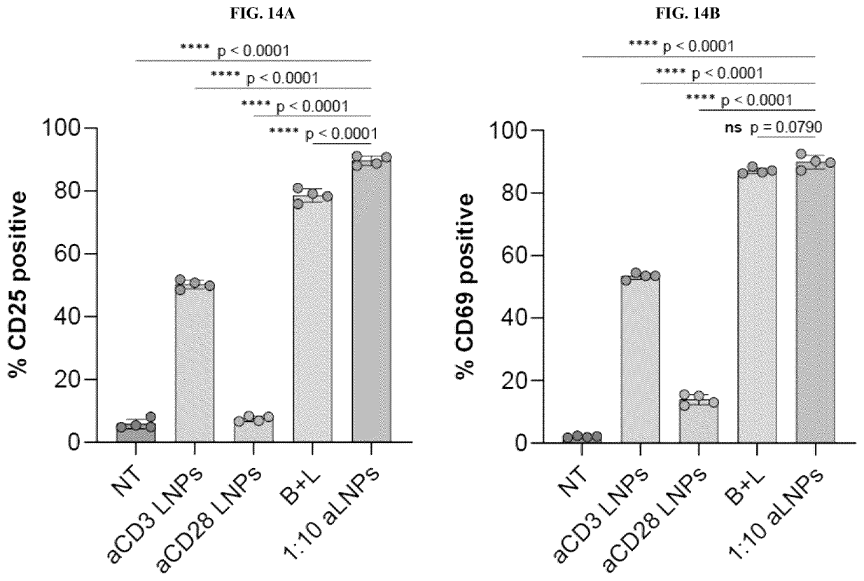

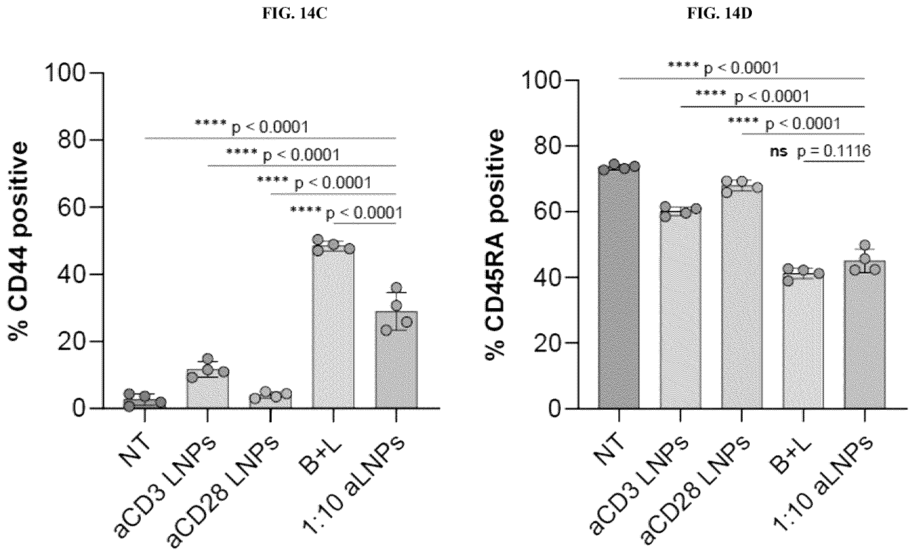

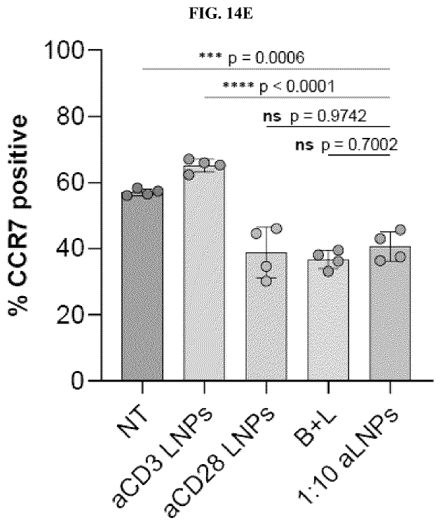

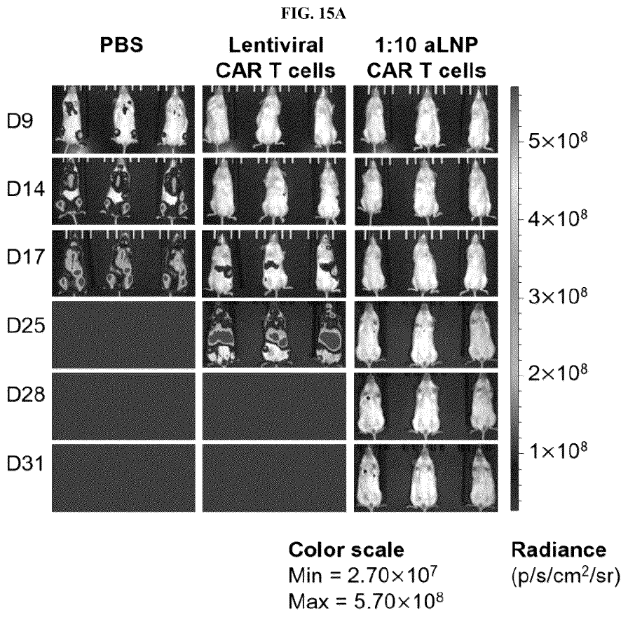

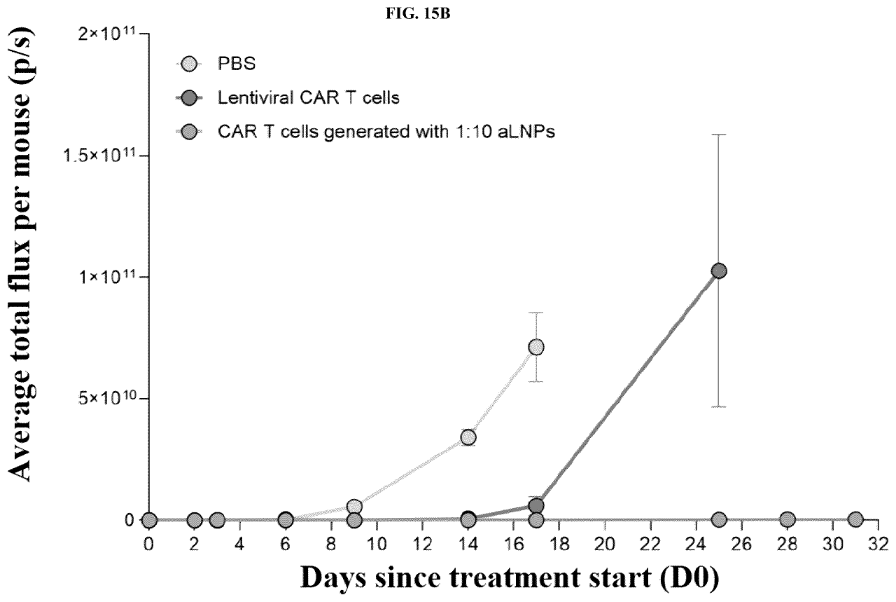

Periodically throughout the treatment, mice were imaged for bioluminescence corresponding to tumor burden and average total flux per mouse was recorded at each imaging timepoint. From day 2 and onward, mice treated with aLNP generated CAR T cells had the lowest tumor burden of the three groups. From day 5 and onward, mice treated with aLNP generated CAR T cells had significantly lower tumor burden than mice treated with PBS, and, from day 6 and onward, mice treated with untransfected T cells and mice treated with PBS had no significant difference in tumor burden. On the day 14, the final day of the experiment, the luminescent signal corresponding to tumor burden in mice treated with aLNP generated CAR T cells was 2.09-fold lower than in mice that received PBS, and 1.84-fold lower than in mice that received untransfected T cells (FIGS. 11B-11C). It was also observed that treatment with aLNP generated CAR T cells extended survival by 6 days compared to treatment with PBS or untransfected T cells, a statistically significant improvement, and administration of untransfected T cells did not significantly extend survival compared to PBS (FIG. 11D). Therefore. CAR T cells generated with aLNPs effectively reduce tumor burden and extend survival in a mouse model of leukemia.

### ENUMERATED EMBODIMENTS

The following exemplary embodiments are provided, the numbering of which is not to be construed as designating levels of importance:

Embodiment 1 provides an immune cell targeted lipid nanoparticle (LNP) comprising:
(a) at least one ionizable lipid;
(b) at least one neutral lipid;
(c) cholesterol and/or a modified derivative thereof,
(d) at least one polymer conjugated lipid and/or a modified derivative thereof; and
(e) a cell targeting domain specific to binding to a surface molecule of a target cell, optionally wherein the cell targeting domain is covalently conjugated to at least one component of the LNP.

Embodiment 2 provides the LNP of Embodiment 1, wherein the at least one ionizable lipid is a compound of Formula (I), or a salt, solvate, stereoisomer, or isotopologue thereof:

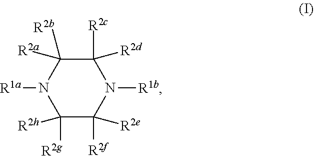

wherein:
R^{1a} and R^{1b} are each independently

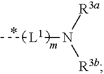

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl; each occurrence of R^{3a} and R^{3b} is independently selected from the group consisting of H, optionally substituted C₁-C₂₈ alkyl, optionally substituted C₂-C₂₉ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
each occurrence of L¹ is independently selected from the group consisting of a bond, optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl; and
m is an integer selected from the group consisting of 1, 2, 3, and 4.

Embodiment 3 provides the LNP of Embodiment 2, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R², R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R², R^{2b}, R^{2c}, R^{2d}, R^{2e}, R^{2f}. R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2C}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 4 provides the LNP of Embodiment 2 or 3, wherein R^{3a} and R^{3b} are each independently selected from the group consisting of H and —CH₂CH(OH)(optionally substituted C₁-C₂₀ alkylenyl)CH₃.

Embodiment 5 provides the LNP of any one of Embodiments 2-4, wherein R^{3a} and R^{3b} are each independently selected from the group consisting of H, —CH₂CH(OH)(CH₂)₉CH₃, —CH₂CH(OH)(CH₂)₁₀CH₃, —CH₂CH(OH)(CH₂)₁₁CH₃, —CH₂CH(OH)(CH₂)₁₂CH₃, and —CH₂CH(OH)(CH₂)₁₃CH₃.

Embodiment 6 provides the LNP of any one of Embodiments 2-5, wherein each occurrence of L¹ is independently selected from the group consisting of a bond, —(CH₂)₁₋₁₀—, —(CH₂)₁₋₃NR⁴—, —(CH₂)₁₋₃O—,

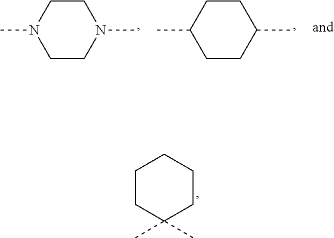

wherein:
each occurrence of R⁴ is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl; and
each occurrence of —CH₂— is independently optionally substituted with at least one selected from the group consisting of C₁-C₁₂ alkyl, C₁-C₁₂ alkoxy, C₁-C₁₂ haloalkyl, C₂-C₁₂ heteroalkyl, and halogen.

Embodiment 7 provides the LNP of any one of Embodiments 2-6, wherein each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aryl, and optionally substituted heteroaryl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″. C₂-C₈ heteroaryl, and phenyl optionally substituted with at least one halogen, wherein each occurrence of R′ and R″ is independently selected from the group consisting of H, C₁-C₆ alkyl, C₃-C₅ cycloalkyl, C₁-C₆ haloalkyl, benzyl, and phenyl.

Embodiment 8 provides the LNP of any one of Embodiments 2-7, wherein R^{1a} and R^{1b} are each independently selected from the group consisting of:

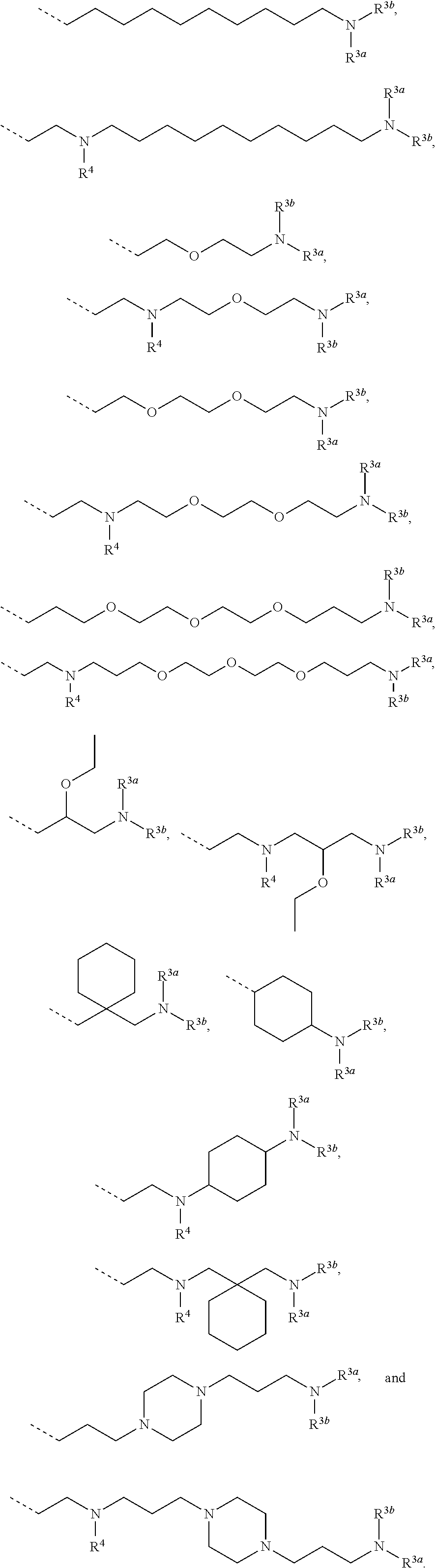

Embodiment 9 provides the LNP of any one of Embodiments 2-8, wherein the at least one ionizable lipid of Formula (I) is selected from the group consisting of:

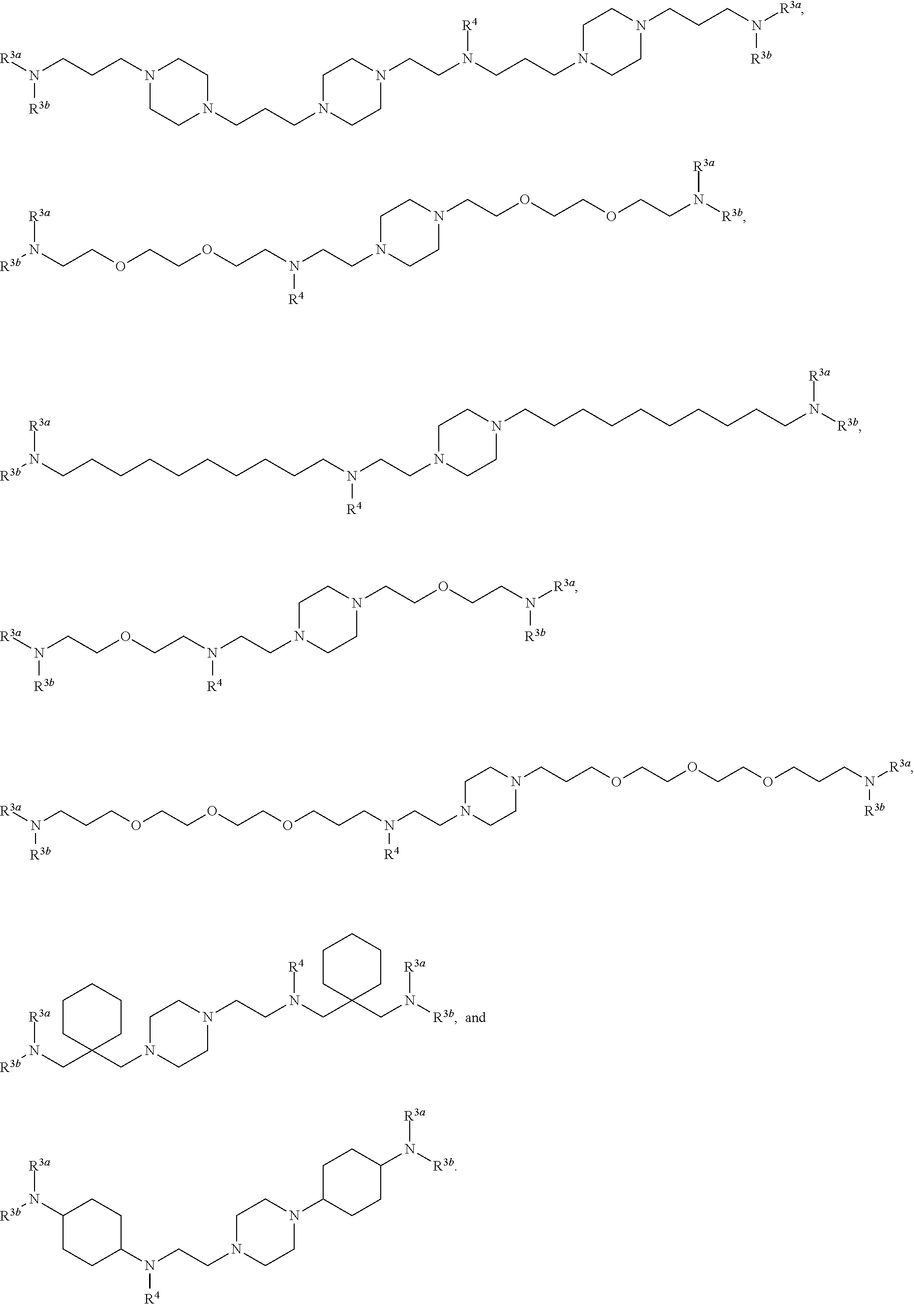

wherein each occurrence of R^{3a}, R^{3b}, and R⁴ is independently selected from the group consisting of H, —CH₂CH(OH)(CH₂)₉CH₃, —CH₂CH(OH)(CH₂)₁₀CH₃, —CH₂CH(OH)(CH₂)₁₁CH₃, —CH₂CH(OH)(CH₂)₁₂CH₃, and —CH₂CH(OH)(CH₂)₁₃CH₃.

Embodiment 10 provides the LNP of any one of Embodiments 1-9, wherein the at least one ionizable lipid comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol).

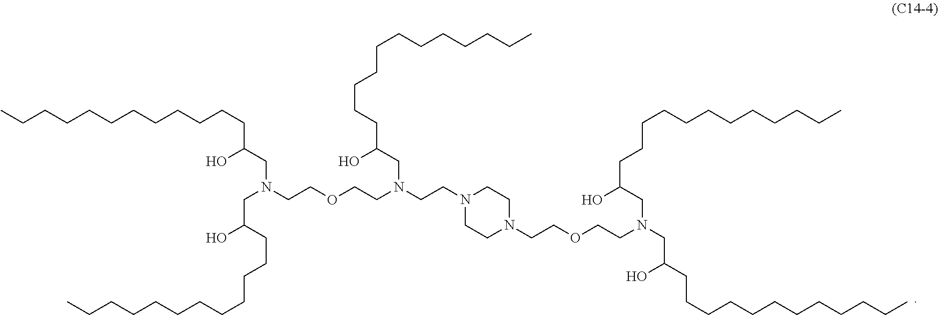 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

Embodiment 11 provides the LNP of any one of Embodiments 1-10, wherein the at least one ionizable lipid comprises about 10 mol % to about 50 mol % of the LNP.

Embodiment 12 provides the LNP of any one of Embodiments 1-11, wherein the at least one ionizable lipid comprises about 40 mol % of the LNP.

Embodiment 13 provides the LNP of any one of Embodiments 1-12, wherein the at least one neutral lipid comprises at least one selected from the group consisting of dioleoylphosphatidylethanolamine (DOPE), distearoylphosphatidylcholine (DSPC), and dioleoylphosphatidylcholine (DOPC).

Embodiment 14 provides the LNP of any one of Embodiments 1-13, wherein the at least one neutral lipid comprises about 5 mol % to about 45 mol % of the LNP.

Embodiment 15 provides the LNP of any one of Embodiments 1-14, wherein the at least one neutral lipid comprises about 30 mol % of the LNP.

Embodiment 16 provides the LNP of any one of Embodiments 1-15, wherein the cholesterol and/or modified derivative thereof comprises about 5 mol % to about 50 mol % of the LNP.

Embodiment 17 provides the LNP of any one of Embodiments 1-16, wherein the cholesterol lipid and/or modified derivative thereof comprises about 25 mol % of the LNP.

Embodiment 18 provides the LNP of any one of Embodiments 1-17, wherein the at least one polymer conjugated lipid and/or modified derivative thereof comprises about 0.5 mol % to about 12.5 mol % of the LNP.

Embodiment 19 provides the LNP of any one of Embodiments 1-18, wherein the at least one polymer conjugated lipid and/or modified derivative thereof comprises about 2.5 mol % of the LNP.

Embodiment 20 provides the LNP of any one of Embodiments 1-19, wherein the at least one polymer conjugated lipid and/or modified derivative thereof comprises a polyethylene glycol (PEG) conjugated lipid.

Embodiment 21 provides the LNP of any one of Embodiments 1-20, wherein the PEG-conjugated lipid comprises 1,2-dimyristoyl-sn-glycero-3-phosphoethanolamine-N-[methoxy(polyethylene glycol)-2000](C14PEG2000).

Embodiment 22 provides the LNP of any one of Embodiments 1-21, wherein the surface molecule of a target cell is a surface antigen of a CD4+ T cell and/or CD8+ T cell.

Embodiment 23 provides the LNP of any one of Embodiments 1-22, wherein the cell targeting domain specific to binding a surface molecule of a target cell is at least one selected from the group consisting of an antibody against CD3 (αCD3) and an antibody against CD28 (αCD28), or a fragment thereof.

Embodiment 24 provides the LNP of any one of Embodiments 1-23, wherein the component to which the cell targeting domain is covalently conjugated is the modified derivative of the polymer conjugated lipid.

Embodiment 25 provides the LNP of Embodiment 24, wherein the covalent conjugation comprises a covalent bond forming reaction selected from the group consisting of a [1,4]-conjugate addition (i.e., Michael addition), [4+2] cycloaddition, [3+2]dipolar cycloaddition, nucleophilic addition, transition metal-catalyzed cross-coupling reaction, carbonyl condensation reaction, and reductive amination.

Embodiment 26 provides the LNP of Embodiment 25, wherein the covalent conjugation reaction comprises a [1,4]-conjugate addition reaction (i.e., Michael addition).

Embodiment 27 provides the LNP of Embodiment 25 or 26, wherein the [1,4]-conjugate addition occurs between the modified derivative of the polymer conjugated lipid which is further conjugated to a maleimide moiety and a cysteine thiol of a polypeptide.

Embodiment 28 provides the LNP of Embodiment 27, wherein the cystine thiol of the polypeptide is derived from a reduced disulfide bridge of a polypeptide selected from the group consisting of an antibody against CD3 (αCD3) and an antibody against CD28 (αCD28), or a fragment thereof.

Embodiment 29 provides the LNP of Embodiment 27 or 28, wherein the LNP has a molar ratio of polymer conjugated lipid and modified derivative of the conjugated lipid further conjugated to a maleimide moiety selected from the group consisting of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10.

Embodiment 30 provides the LNP of any one of Embodiments 1-24, wherein the modified derivative of the polymer conjugated lipid is a compound of Formula (II), or a salt, solvate, stereoisomer, or isotopologue thereof:

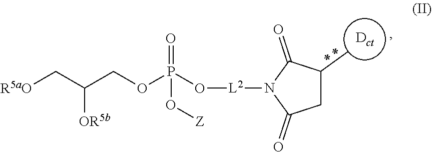

wherein:
R^{5a} and R^{5b} are each independently selected form the group consisting of —C(═O)(optionally substituted C₁-C₂₈ alkyl), —C(═O)(optionally substituted C₂-C₂₈ alkenyl), —C(═O)(optionally substituted C₂-C₂₈ alkynyl), optionally substituted C₁-C₂₈ alkyl, optionally substituted C₂-C₂₈ alkenyl, and optionally substituted C₂-C₂₈ alkynyl;
Z is a monovalent cation;
L² comprises n units of

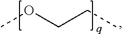

[symbol]

units of

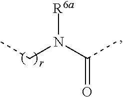

and p units of

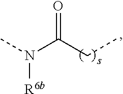

wherein each ---- in L² is a C—O or C—N bond;
D_{ct} is a cell targeting domain comprising an antibody against CD3 or CD28,
wherein [symbol] is C—S bond;
R^{6a} and R^{6b} are ‘each’ independently selected from the group consisting of H and C₁-C₆ alkyl;
n, [symbol] , and p are each independently 1, 2, 3, 4, or 5;
q is an integer ranging from 1 to 100; and
r and s are each independently an integer ranging from 1 to 10.

Embodiment 31 provides the LNP of Embodiment 30, wherein R^{5a} and R^{5b} are each independently C(═O)(C₅-C₂₀ alkyl), optionally wherein R^{5a} and R^{5b} are each independently C(═O)(CH₂)₁₆CH₃.

Embodiment 32 provides the LNP of Embodiment 30 or 31, wherein Z is NH⁴⁺.

Embodiment 33 LNP of any one of Embodiments 30-32, wherein L² is

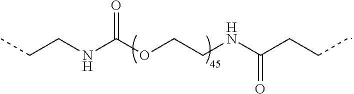

Embodiment 34 provides the LNP of any one of Embodiments 30-33, wherein the compound of formula (II) is:

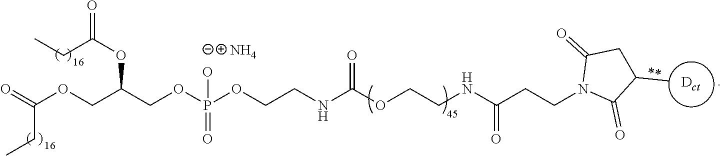

Embodiment 35 provides the LNP of any one of Embodiments 30-34, wherein Det comprises at least one of an antibody of CD3 (αCD3) and an antibody of CD28 (αCD28).

Embodiment 36 provides the LNP of Embodiment 35, wherein the antibody of CD3 and the antibody of CD28 have a ratio ranging from about 100:1 to about 1:100 (αCD3:αCD28).

Embodiment 37 provides the LNP of any one of Embodiments 31-36, wherein (d) comprises the polymer conjugated lipid and the compound of formula (II), wherein the polymer conjugated lipid and the compound of formula (II) have a molar ratio of about 4.9:0.1, 4.8:0.2, 4.7:0.3, 4.6:0.4, 4.5:0.5, 4.4:0.6, 4.3:0.7, 4.2:0.8, 4.1:0.9, 4.0:1.0, 3.9:1.1, 3.8:1.2, 3.7:1.3, 3.6:1.4, 3.5:1.5, 3.4:1.6, 3.3:1.7, 3.2:1.8, 3.1:1.9, 3.0:2.0, 2.9:2.1, 2.8:2.2, 2.7:2.3, 2.6:2.4, 2.5:2.5, 2.4:2.6, 2.3:2.7, 2.2:2.8, 2.1:2.9, 2.0:3.0, 1.9:3.1, 1.8:3.2, 1.7:3.3, 1.6:3.4, 1.5:3.5, 1.4:3.6, 1.3:3.7, 1.2:3.8, 1.1:3.9, 1.0:4.0, 0.9:4.1, 0.8:4.2, 0.7:4.3, 0.6:4.4, 0.5:4.5, 0.4:4.6, 0.3:4.7, 0.2:4.8, or about 0.1:4.9.

Embodiment 38 provides the LNP of any one of Embodiments 31-37, wherein the LNP has a molar ratio of (a):(b):(c):(d) of about 40:30:25:2.5 or about 41:30.8:25.6:2.5, optionally wherein (d) comprises the polymer conjugated lipid and the compound of formula (II) having a ratio of about 2.1:0.4.

Embodiment 39 provides the LNP of any one of Embodiments 1-38, wherein the LNP further comprises at least one cargo selected from the group consisting of a nucleic acid molecule and a therapeutic agent.

Embodiment 40 provides the LNP of Embodiment 39, wherein the therapeutic agent is at least one selected from the group consisting of a small molecule, a protein, and an antibody.

Embodiment 41 provides the LNP of Embodiment 39, wherein the LNP comprises a nucleic acid molecule.

Embodiment 42 provides the LNP of Embodiment 41, wherein the nucleic acid molecule is a DNA molecule or an RNA molecule.

Embodiment 43 provides the LNP of Embodiment 41 or 42, wherein the nucleic acid molecule is selected from the group consisting of cDNA, mRNA, miRNA, siRNA, modified RNA, antagomir, antisense molecule, and a targeted nucleic acid, or any combination thereof.

Embodiment 44 provides the LNP of any one of Embodiments 41-43, wherein the nucleic acid molecule encodes a chimeric antigen receptor (CAR).

Embodiment 45 provides the LNP of Embodiment 44, wherein the CAR is specific for binding to a surface antigen of a pathogenic cell or a tumor cell.

Embodiment 46 provides the LNP of Embodiment 45, wherein the surface antigen is selected from the group consisting of CD4, CD8, CD1, CD2, CD3, CD5, CD7, CD16, CD19, CD20, CD22, CD25, CD26, CD27, CD28, CD30, CD33, CD38, CD39, CD40L, CD44, CD45, CD62L, CD69, CD73, CD80, CD83, CD86, CD95, CD103, CD119, CD123, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, CCR7, k light chain, ROR1, ErbB2, ErbB3, ErbB4, EGFR vIII, carcinoembryonic antigen, EGP2, EGP40, mesothelin, TAG72, PSMA, NKG2D ligands, B7-H6, IL13R-α2, MUC1, VEGF-A, Tem8, FAP, EphA2, HER2, MUC16, CA9, GD2, GD3, HMW-MAA, CD171, Lewis Y, G250/CALX, HLA-AI MAGE A1, HAL-A2 NY-ESO-1, PSC1, folate receptor-α, 8H9, NCAM, VEGF, 5T4, Fetal AchR, NKG2D ligands, TEM1, and TEM8.

Embodiment 47 provides the LNP of any one of Embodiments 41-43, wherein the nucleic acid molecule encodes at least one selected from the group consisting of mRNA and sgRNA.

Embodiment 48 provides the LNP of Embodiment 47, wherein the mRNA encodes a therapeutic protein, optionally wherein the therapeutic protein is a CRISPR-associated protein, and optionally wherein the CRISPR-associated protein is CRISPR-associated protein 9 (Cas9).

Embodiment 49 provides the LNP of Embodiment 39 or 40, wherein the therapeutic agent is a CRISPR-associated protein, optionally wherein the CRISPR-associated protein is CRISPR-associated protein 9 (Cas9).

Embodiment 50 provides a pharmaceutical composition comprising the lipid nanoparticle (LNP) of any one of Embodiments 1-49 and at least one pharmaceutically acceptable carrier.

Embodiment 51 provides a method of treating, preventing, and/or ameliorating cancer in a subject, the method comprising administering to the subject the lipid nanoparticle (LNP) of any one of Embodiments 1-49 and/or the pharmaceutical composition of Embodiment 50.

Embodiment 52 provides the method of Embodiment 51, wherein the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer.

Embodiment 53 provides the method of Embodiment 51, wherein the cancer is at least one selected from the group consisting of leukemia, non-Hodgkin lymphoma, Hodgkin lymphoma, multiple myeloma, myelodysplastic syndromes (MDS), and myeloproliferative neoplasms (MPNs).

Embodiment 54 provides the method of any one of Embodiments 51-53, wherein the subject is further administered at least one additional agent or therapy useful for treating, preventing, and/or ameliorating cancer in a subject.

Embodiment 55 provides the method of Embodiment 54, wherein the at least one additional agent is selected from the group consisting of a small molecule anti-cancer agent and an antibody anti-cancer agent.

Embodiment 56 provides the method of any one of Embodiments 51-55, wherein the subject is a mammal.

Embodiment 57 provides the method of Embodiment 56, wherein the mammal is a human.

Embodiment 58 provides a method of preparing a modified immune cell or precursor thereof, comprising contacting an immune cell or precursor thereof with the lipid nanoparticle (LNP) of any one of Embodiments 1-49.

Embodiment 59 provides the method of Embodiment 58, wherein the modified immune cell or precursor cell thereof is selected from the group consisting of an αβ T cell, a γδ T cell, a CD8+ T cell, a CD4+ helper T cell, a CD4+ regulatory T cell, an NK T cell, an NK cell, and any combination thereof.

Embodiment 60 provides the method of Embodiment 59, wherein the modified immune cell or precursor cell thereof is a T cell.

Embodiment 61 provides the method of any one of Embodiments 58-60, wherein the modified immune cell or precursor cell thereof is a CD4+ T cell and/or CD8+ T cell.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An immune cell targeted lipid nanoparticle (LNP) comprising:
(a) at least one ionizable lipid;
(b) at least one neutral lipid;
(c) cholesterol or a modified derivative thereof;
(d) at least one polymer conjugated lipid or a modified derivative thereof; and
(e) a cell targeting domain specific to binding to a surface molecule of a target cell, optionally wherein the cell targeting domain is covalently conjugated to at least one component of the LNP.

2. The LNP of claim 1, wherein the at least one ionizable lipid is a compound of Formula (I), or a salt, solvate, stereoisomer, or isotopologue thereof: 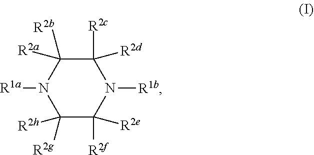
wherein:
R^{1a} and R^{1b} are each independently 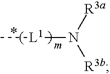
R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl;
each occurrence of R^{3a} and R^{3b} is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
each occurrence of L¹ is independently selected from the group consisting of a bond, optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl; and
m is selected from the group consisting of 1, 2, 3, and 4.

3. The LNP of claim 2, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

4. The LNP of claim 2, wherein at least one of the following applies:
(a) R^{3a} and R^{3b} are each independently selected from the group consisting of H and —CH₂CH(OH)(optionally substituted C₁-C₂₀ alkylenyl)CH₃, optionally wherein R^{3a} and R^{3b} are each independently selected from the group consisting of H, —CH₂CH(OH)(CH₂)₉CH₃, —CH₂CH(OH)(CH₂)₁₀CH₃, —CH₂CH(OH)(CH₂)₁₁CH₃, —CH₂CH(OH)(CH₂)₁₂CH₃, and —CH₂CH(OH)(CH₂)₁₃CH₃;
(b) each occurrence of L¹ is independently selected from the group consisting of a bond, —(CH₂)₁₋₁₀—, —(CH₂)₁₋₃NR⁴—, —(CH₂)₁₋₃O—, 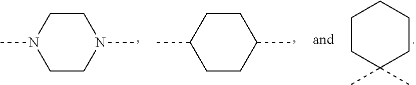
wherein:
each occurrence of R⁴ is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl; and
each occurrence of —CH₂— is independently optionally substituted with at least one selected from the group consisting of C₁-C₁₂ alkyl, C₁-C₁₂ alkoxy, C₁-C₁₂ haloalkyl, C₂-C₁₂ heteroalkyl, and halogen;
(c) R^{1a} and R^{1b} are each independently selected from the group consisting of: 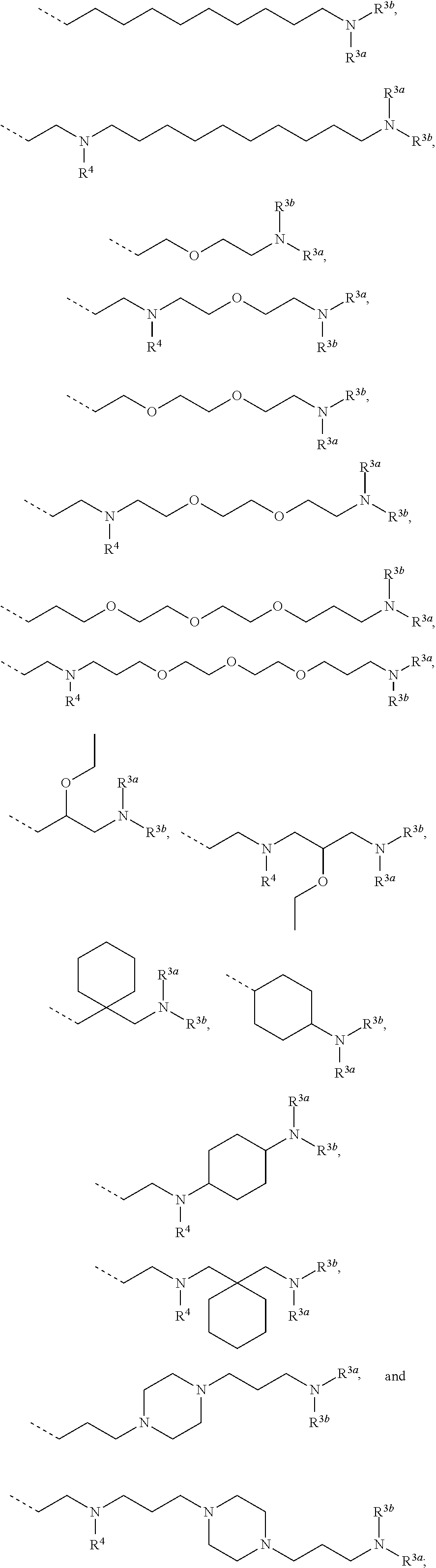
(d) each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aryl, and optionally substituted heteroaryl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

5-8. (canceled)

9. The LNP of claim 2, wherein the at least one ionizable lipid of Formula (I) is selected from the group consisting of: 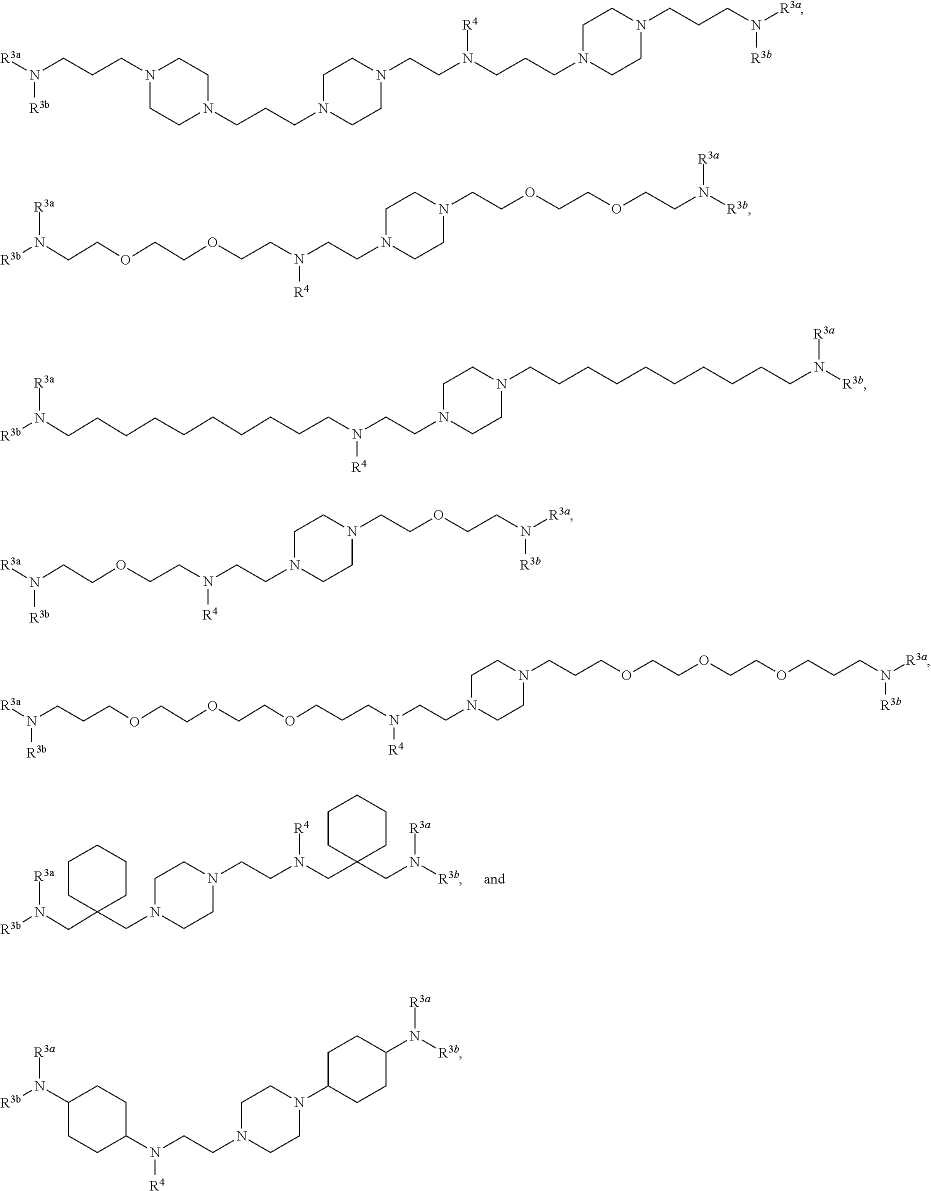
wherein each occurrence of R^{3a}, R^{3b}, and R⁴ is independently selected from the group consisting of H, —CH₂CH(OH)(CH₂)₉CH₃, —CH₂CH(OH)(CH₂)₁₀CH₃, —CH₂CH(OH)(CH₂)₁₁CH₃, —CH₂CH(OH)(CH₂)₁₂CH₃, and —CH₂CH(OH)(CH₂)₁₃CH₃.

10. The LNP of claim 1, wherein the at least one ionizable lipid comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(8etradecane-2-ol):

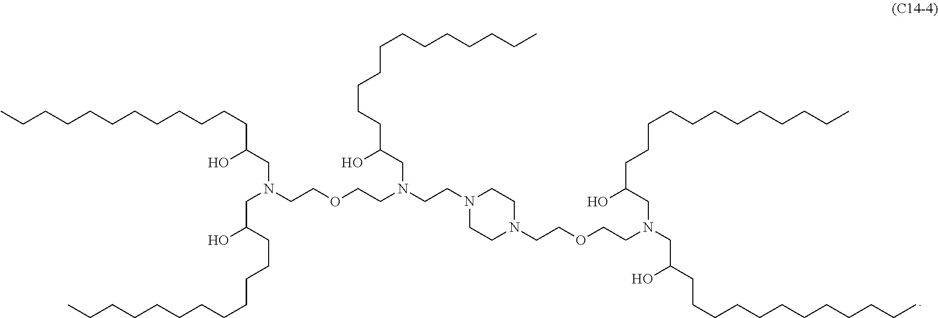 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

11. The LNP of claim 1, wherein at least one of the following applies:
(a) the at least one ionizable lipid comprises about 10 mol % to about 50 mol % of the LNP, optionally wherein at least one ionizable lipid comprises about 40 mol % of the LNP;
(b) the at least one neutral lipid comprises at least one selected from the group consisting of dioleoylphosphatidylethanolamine (DOPE), distearoylphosphatidylcholine (DSPC), and dioleoylphosphatidylcholine (DOPC);
(c) the at least one neutral lipid comprises about 5 mol % to about 45 mol % of the LNP, optionally wherein the at least one neutral lipid comprises about 30 mol % of the LNP;
(d) the cholesterol or modified derivative thereof comprises about 5 mol % to about 50 mol % of the LNP, optionally wherein the cholesterol lipid or modified derivative thereof comprises about 25 mol % of the LNP; and
(e) the at least one polymer conjugated lipid or modified derivative thereof comprises about 0.5 mol % to about 12.5 mol % of the LNP, optionally wherein the at least one polymer conjugated lipid or modified derivative thereof comprises about 2.5 mol % of the LNP; and
(f) the at least one polymer conjugated lipid or modified derivative thereof comprises a polyethylene glycol (PEG) conjugated lipid, optionally wherein the PEG-conjugated lipid comprises 1,2-dimyristoyl-sn-glycero-3-phosphoethanolamine-N-[methoxy(polyethylene glycol)-2000](C14PEG2000).

12-21. (canceled)

22. The LNP of claim 1, wherein at least one of the following applies:
(a) the surface molecule of a target cell is a surface antigen of at least one of a CD4+ T cell and/er CD8+ T cell;
(b) the cell targeting domain specific to binding a surface molecule of a target cell is at least one selected from the group consisting of an antibody against CD3 (αCD3) and an antibody against CD28 (αCD28), or a fragment thereof; and
(c) the component to which the cell targeting domain is covalently conjugated is the modified derivative of the polymer conjugated lipid.

23-24. (canceled)

25. The LNP of claim 22, wherein the covalent conjugation comprises a covalent bond forming reaction selected from the group consisting of a [1,4]-conjugate addition (i.e., Michael addition), [4+2] cycloaddition, [3+2]dipolar cycloaddition, nucleophilic addition, transition metal-catalyzed cross-coupling reaction, carbonyl condensation reaction, and reductive amination.

26. (canceled)

27. The LNP of claim 25, wherein at least one of the following applies:
(a) the [1,4]-conjugate addition occurs between the modified derivative of the polymer conjugated lipid which is further conjugated to a maleimide moiety and a cysteine thiol of a polypeptide;
(b) the cystine thiol of the polypeptide is derived from a reduced disulfide bridge of a polypeptide selected from the group consisting of an antibody against CD3 (αCD3) and an antibody against CD28 (αCD28), or a fragment thereof; and
(c) the LNP has a molar ratio of polymer conjugated lipid and modified derivative of the conjugated lipid further conjugated to a maleimide moiety selected from the group consisting of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10.

28-29. (canceled)

30. The LNP of claim 1, wherein the modified derivative of the polymer conjugated lipid is a compound of Formula (II), or a salt, solvate, stereoisomer, or isotopologue thereof: 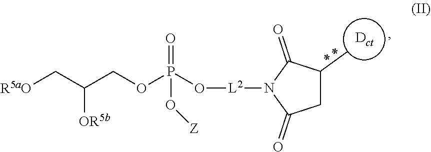
wherein:
R^{5a} and R^{5b} are each independently selected form the group consisting of —C(═O)(optionally substituted C₁-C₂₈ alkyl), —C(═O)(optionally substituted C₂-C₂₈ alkenyl), —C(═O)(optionally substituted C₂-C₂₈ alkynyl), optionally substituted C₁-C₂₈ alkyl, optionally substituted C₂-C₂₈ alkenyl, and optionally substituted C₂-C₂₈ alkynyl;
Z is a monovalent cation;
L² comprises n units of 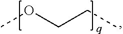 [symbol]
units of 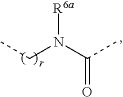
and p units of 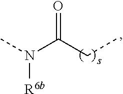
wherein each ---- in L² is a C—O or C—N bond;
D_{ct} is a cell targeting domain comprising an antibody against CD3 or CD28, wherein [symbol] is C—S bond;
R^{6a} and R^{6b} are each independently selected from the group consisting of H and C₁-C₆ alkyl;
n, [symbol] and p are each independently 1, 2, 3, 4, or 5;
q is an integer ranging from 1 to 100; and
r and s are each independently selected from the group consisting of 1, 2, 3, 4, 5, 6, 7, 8, 9, and 10 .

31. The LNP of claim 30, wherein at least one of the following applies:
(a) R^{5a} and R^{5b} are each independently C(═O)(C₅-C₂₀ alkyl), optionally wherein R^{5a} and R^{5b} are each independently C(═O)(CH₂)₁₆CH₃;
(b) Z is NH₄⁺;
(c) L² is 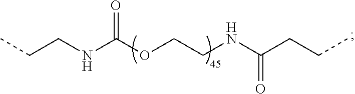
(d) the compound of formula (II) is 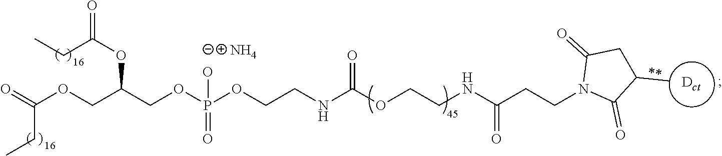
and
(e) D_{ct} comprises at least one of an antibody of CD3 (αCD3) and an antibody of CD28 (αCD28), optionally wherein the antibody of CD3 and the antibody of CD28 have a ratio ranging from about 100:1 to about 1:100 (αCD3:αCD28).

32-36. (canceled)

37. The LNP of claim 30, wherein (d) comprises the polymer conjugated lipid and the compound of formula (II), wherein the polymer conjugated lipid and the compound of formula (II) have a molar ratio of about 4.9:0.1, 4.8:0.2, 4.7:0.3, 4.6:0.4, 4.5:0.5, 4.4:0.6, 4.3:0.7, 4.2:0.8, 4.1:0.9, 4.0:1.0, 3.9:1.1, 3.8:1.2, 3.7:1.3, 3.6:1.4, 3.5:1.5, 3.4:1.6, 3.3:1.7, 3.2:1.8, 3.1:1.9, 3.0:2.0, 2.9:2.1, 2.8:2.2, 2.7:2.3, 2.6:2.4, 2.5:2.5, 2.4:2.6, 2.3:2.7, 2.2:2.8, 2.1:2.9, 2.0:3.0, 1.9:3.1, 1.8:3.2, 1.7:3.3, 1.6:3.4, 1.5:3.5, 1.4:3.6, 1.3:3.7, 1.2:3.8, 1.1:3.9, 1.0:4.0, 0.9:4.1, 0.8:4.2, 0.7:4.3, 0.6:4.4, 0.5:4.5, 0.4:4.6, 0.3:4.7, 0.2:4.8, or about 0.1:4.9, optionally wherein the LNP has a molar ratio of (a):(b):(c): (d) of about 40:30:25:2.5 or about 41:30.8:25.6:2.5, optionally wherein (d) comprises the polymer conjugated lipid and the compound of formula (II) having a ratio of about 2.1:0.4.

38. (canceled)

39. The LNP of claim 1, wherein the LNP further comprises at least one cargo selected from the group consisting of a nucleic acid molecule and a therapeutic agent, optionally wherein at least one of the following applies:
(a) the therapeutic agent is at least one selected from the group consisting of a small molecule, a protein, and an antibody; and
(b) the nucleic acid molecule is a DNA molecule or an RNA molecule, optionally wherein the DNA molecule or RNA molecule is of selected from the group consisting of cDNA, mRNA, miRNA, siRNA, modified RNA, antagomir, antisense molecule, and a targeted nucleic acid, or any combination thereof.

40-43. (canceled)

44. The LNP of claim 39, wherein at least one of the following applies:
(a) the nucleic acid molecule encodes a chimeric antigen receptor (CAR), optionally wherein the CAR is specific for binding to a surface antigen of a pathogenic cell or a tumor cell, and optionally wherein the surface antigen is selected from the group consisting of CD4, CD8, CD1, CD2, CD3, CD5, CD7, CD16, CD19, CD20, CD22, CD25, CD26, CD27, CD28, CD30, CD33, CD38, CD39, CD40L, CD44, CD45, CD62L, CD69, CD73, CD80, CD83, CD86, CD95, CD103, CD119, CD123, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, CCR7, k light chain, ROR1, ErbB2, ErbB3, ErbB4, EGFR vIII, carcinoembryonic antigen, EGP2, EGP40, mesothelin, TAG72, PSMA, NKG2D ligands, B7-H6, IL13R-α2, MUC1, VEGF-A, Tem8, FAP, EphA2, HER2, MUC16, CA9, GD2, GD3, HMW-MAA, CD171, Lewis Y, G250/CALX, HLA-AI MAGE A1, HAL-A2 NY-ESO-1, PSC1, folate receptor-α, 8H9, NCAM, VEGF, 5T4, Fetal AchR, NKG2D ligands, TEM1, and TEM8; and
(b) the nucleic acid molecule encodes at least one selected from the group consisting of mRNA and sgRNA, optionally wherein the mRNA encodes a therapeutic protein, optionally wherein the therapeutic protein is a CRISPR-associated protein, optionally wherein the CRISPR-associated protein is CRISPR-associated protein 9 (Cas9), and optionally wherein the therapeutic agent is a CRISPR-associated protein.

45-49. (canceled)

50. A pharmaceutical composition comprising the lipid nanoparticle (LNP) of claim 1 and at least one pharmaceutically acceptable carrier.

51. A method of treating, preventing, or ameliorating cancer in a subject, the method comprising administering to the subject the lipid nanoparticle (LNP) of claim 1 or a pharmaceutical composition thereof.

52. The method of claim 51, wherein at least one of the following applies:
(a) the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer, optionally wherein the cancer is at least one selected from the group consisting of leukemia, non-Hodgkin lymphoma, Hodgkin lymphoma, multiple myeloma, myelodysplastic syndromes (MDS), and myeloproliferative neoplasms (MPNs);
(b) the subject is further administered at least one additional agent or therapy useful for treating, preventing, or ameliorating cancer in a subject, optionally wherein the at least one additional agent is selected from the group consisting of a small molecule anti-cancer agent and an antibody anti-cancer agent; and
(c) the subject is a mammal, optionally wherein the mammal is a human.

53-57. (canceled)

58. A method of preparing a modified immune cell or precursor thereof, comprising contacting an immune cell or precursor thereof with the lipid nanoparticle (LNP) of claim 1.

59. The method of claim 58, wherein at least one of the following applies:
(a) the modified immune cell or precursor cell thereof is selected from the group consisting of an αβ T cell, a γδ T cell, a CD8+ T cell, a CD4+ helper T cell, a CD4+ regulatory T cell, an NK T cell, an NK cell, and any combination thereof; and
(b) the modified immune cell or precursor cell thereof is a T cell, optionally wherein the T cell is at least one of a CD4+ T cell and a CD8+ T cell.

60-61. (canceled)
